(19) United States
(12) Patent Application Publication
(10) Pub. No.: US 2012/0131687 A1
(43) Pub. Date: May 24, 2012

(54) AGENT THAT MODULATES PHYSIOLOGICAL CONDITION OF PESTS, INVOLVED IN INSECT VOLTAGE-GATED POTASSIUM CHANNEL ACTIVITY

(75) Inventors: Junko Otsuki, Osaka (JP); Marc Logghe, Sint-Denijs-Westrem (BE); Titus Kaletta, Nieder-Olm (BE); Wendy Maddelein, Gijzenzele (BE)

(73) Assignee: SUMITOMO CHEMICAL COMPANY, LTD., Chuo-ku, Tokyo (JP)

(21) Appl. No.: 13/120,496

(22) PCT Filed: Sep. 24, 2009
(86) PCT No.: PCT/JP2009/067131

§ 371 Date: Sep. 6, 2011

(30) Foreign Application Priority Data
Sep. 24, 2008 (JP) 2008-243846

(51) Int. Cl. G01N 21/75; C07K 14/435; C07C 211/63; A01N 33/12; C07C 309/30; A01P 5/00; A01P 7/04; A01P 7/02; A01K 67/033; C12P 19/34; C12N 1/21; C12N 5/10; C12N 15/63; C12P 21/02; G01N 27/00; G09G 5/00; C12N 15/12

(52) U.S. Cl. 800/3; 536/23.5; 530/350; 564/289; 514/643; 562/84; 800/13; 435/91.2; 435/358; 435/252.33; 435/369; 435/348; 435/455; 435/471; 435/69.1; 800/4; 324/71.1; 345/156

(57) ABSTRACT

The present invention provides an agent that modulates physiological condition of pests, wherein the agent has an ability to modulate the activity of an insect voltage-gated potassium channel; a method for assaying pesticidal activity of a test substance, which comprises measuring the activity of a voltage-gated potassium channel in a reaction system in which the voltage-gated potassium channel contacts with a test substance, and so on.

### TECHNICAL FIELD

The present invention relates to an agent that modulates physiological condition of pests, involved in insect voltage-gated potassium channel activity, or the like.

### BACKGROUND ART

Voltage-gated potassium channels are membrane proteins which respond to membrane potential to selectively permeate potassium ions, and widely exist in animals, plants, and microorganisms, and involve various molecular species. Depending on the characteristics of the structure and the physiological function, the voltage-gated potassium channels are classified into a plurality of families, that is, Shaker type, EAG (ether-a-go-go) type, KvLQT and so on (Gutman et al., Pharmacol. Reviews, 55 (4):583-586, 2003).

Seizure is an insect homolog of human ERG (hERG) and is a member of the EAG family of the voltage-gated potassium channels. The EAG family consists of the three subfamilies EAG (ether-a-go-go), ERG (EAG-related gene) and ELK (EAG-like gene). Nomenclature according to the International Union of Basic and Clinical Pharmacology (IUPHAR): EAG is Kv10.x, ERG is Kv11.x and ELK is Kv12.x. The human EAG family is also known as KCNH family.

The seizure gene product is a voltage-gated potassium channel subunit (VGKC) from the six-transmembrane domain super family of voltage-gated ion channels. In general terms, reduction in potassium channel function results in hyper-excitability of cells and activation of potassium channel function results in hypo-excitability of cells.

Drosophila melanogaster seizure null mutants exhibit a hyperactive phenotype. All null mutants described by Wang et al. (J. Neurosci., 17(3):882-890, 1997) show an additional phenotype, paralysis, at high temperature conditions.

Caenorhabditis elegans loss-of-function mutants of the seizure orthologue unc-103 do not exhibit marked defects but gain-of-function (channel activating) mutations lead to severe movement and egg-laying defects (WormBase, www.wormbase.org/). These data indicate that over-activation of seizure-like potassium channels also leads to severe neuromuscular defects.

Discovery of agricultural chemicals has traditionally been based on a random screening process, often directly testing the effects of specific chemicals on whole organisms (pests), such as insects, fungi and plants and determining biological activity. Once chemical compounds with the appropriate biological activity are discovered, more intense research is required to specifically determine the mode of action or site of action of these compounds at the molecular level, in order to predict safety and environmental load of these compounds.

### DISCLOSURE OF INVENTION

The present invention describes a more target-based approach of screening agricultural chemicals, whereby compounds are screened against a specific target that has been identified as biologically and/or physiologically relevant with intent of chemically interfering with the target site to control insects or other pest organisms.

Specifically, the present invention describes that an agent that modulates physiological condition of pests and having an ability to modulate the activity of an insect voltage-gated potassium channel is useful to control pests.

That is, the present invention provides:

1. An agent that modulates physiological condition of pests, wherein the agent has an ability to modulate the activity of an insect voltage-gated potassium channel;
2. The agent according to item 1, wherein the voltage-gated potassium channel is an insect voltage-gated potassium channel from the EAG family;
3. The agent according to item 2, wherein the voltage-gated potassium channel is an insect ERG-type voltage-gated potassium channel;
4. The agent according to item 3, wherein the voltage-gated potassium channel is a cotton aphid ERG-type voltage-gated potassium channel;
5. The agent according to item 1, wherein the agent is a pesticidal agent;
6. The agent according to item 1, wherein the ability to modulate the activity of an insect voltage-gated potassium channel is an ability to modulate the feeding behavior activity of a nematode that expresses the insect voltage-gated potassium channel in a form functional as an ion channel, or an ability to modulate the electrophysiological activity of the insect voltage-gated potassium channel in a cell that expresses the voltage-gated potassium channel in a form functional as an ion channel;
7. A pesticidal agent which comprises a substance that has an ability to modulate the activity of an insect voltage-gated potassium channel or an agriculturally acceptable salt of the substance as an active ingredient;
8. The pesticidal agent according to item 7, wherein the substance has an ability to modulate the feeding behavior activity of a nematode which expresses the insect voltage-gated potassium channel in a form functional as an ion channel;
9. The pesticidal agent according to item 8, wherein the substance has an ability to inhibit the feeding behavior activity of a nematode that expresses the insect voltage-gated potassium channel in a form functional as an ion channel, wherein in the presence of the substance of 30 micro M or more the feeding behavior activity is lower than that in the absence of the substance;
10. The pesticidal agent according to item 8, wherein the substance has an ability to inhibit the feeding behavior activity of a nematode that expresses the insect voltage-gated potassium channel in a form functional as an ion channel, wherein an effective concentration of the substance at which the feeding behavior activity is reduced by 50% is 100 μM or lower;
11. The pesticidal agent according to item 8, wherein the substance has an ability to activate the feeding behavior activity of a nematode that expresses the insect voltage-gated potassium channel in a form functional as an ion channel, wherein in the presence of said substance of 30 micro M or more the feeding behavior activity is higher than that in the absence of the substance;
12. The pesticidal agent according to item 8, wherein the substance has an ability to activate the feeding behavior activity of a nematode that expresses the insect voltage-gated potassium channel in a form functional as an ion channel, wherein an effective concentration of the substance at which the feeding behavior activity is increased by 50% is 100 μM or lower;
13. The pesticidal agent according to item 7, wherein the substance has an ability to modulate the electrophysiological activity of the insect voltage-gated potassium channel in a cell that expresses the voltage-gated potassium channel in a form functional as an ion channel;
14. A method for assaying the pesticidal activity of a test substance, comprising:

(1) a first step of measuring the feeding behavior activity of a nematode that expresses a voltage-gated potassium channel selected from among the following group A in a form functional as an ion channel in a system in which the nematode contacts with a test substance, and

(2) a second step of assessing the pesticidal activity of the test substance based on a difference obtained by comparing the feeding behavior activity measured in the first step with the feeding behavior activity of the nematode in a system containing no test substance;

### <Group A>

(a) a protein comprising the amino acid sequence of SEQ ID NO: 1, 3, 5 or 7, or a partial sequence thereof functioning as an ion channel,

(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,

(c) a protein having the voltage-gated potassium channel activity and comprising an amino acid sequence having 60% or more sequence identity with the amino acid sequence of SEQ ID NO: 1, 3, 5 or 7,

(d) a protein comprising an amino acid sequence encoded by the nucleotide sequence of SEQ ID NO: 2, 4, 6 or 8,

(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,

(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,

(g) a protein comprising an amino acid sequence of an insect ERG-type voltage-gated potassium channel,

(h) a protein comprising an amino acid sequence of a cotton aphid ERG-type voltage-gated potassium channel, and

(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and

SEQ ID NO: 12 or 14 is a nucleotide sequence of PCR 3′ primer; 15. A method for assaying the pesticidal activity of a test substance, comprising:

(1) a first step of measuring the electrophysiological activity of a voltage-gated potassium channel of a cell that expresses a voltage-gated potassium channel selected from among the following group A in a form functional as an ion channel in a system in which the cell contact with a test substance, and

(2) a second step of assessing the pesticidal activity of the test substance based on a difference obtained by comparing the electrophysiological activity of the voltage-gated potassium channel measured in the first step with the electrophysiological activity of the voltage-gated potassium channel of the cell in a system containing no test substance:

### <Group A>

(a) a protein comprising the amino acid sequence of SEQ ID NO: 1, 3, 5 or 7, or a partial sequence thereof functioning as an ion channel,

(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,

(c) a protein having the voltage-gated potassium channel activity and comprising an amino acid sequence having 60% or more sequence identity with the amino acid sequence of SEQ ID NO: 1, 3, 5 or 7,

(d) a protein comprising an amino acid sequence encoded by the nucleotide sequence of SEQ ID NO: 2, 4, 6 or 8,

(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,

(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,

(g) a protein comprising an amino acid sequence of an insect ERG-type voltage-gated potassium channel,

(h) a protein comprising an amino acid sequence of a cotton aphid ERG-type voltage-gated potassium channel, and

(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and SEQ ID NO: 12 or 14 is a nucleotide sequence of PCR 3′ primer;

16. A method for screening a pesticidal substance, which comprises selecting a substance having the pesticidal activity that is evaluated by the method of item 14 or 15;
17. A pesticidal agent which comprises a substance selected by the method of item 16 or an agriculturally acceptable salt thereof as an active ingredient;
18. A method for controlling pests which comprises applying an effective amount of the pesticidal agent of any of items 7 to 13 or 17 to the pest, habitat of the pest or plant to be protected from the pest;
19. A method for controlling pests which comprises:

identifying a substance having the pesticidal activity that is evaluated by the method of item 14 or 15, and

bringing the identified pesticidal substance into contact with the pest;

20. A voltage-gated potassium channel comprising an amino acid sequence selected from among the following group B:

### <Group B>

(a) the amino acid sequence of SEQ ID NO: 1, 3, 5 or 7,

(b) an amino acid sequence which has the voltage-gated potassium channel activity and which has deletion, addition or substitution of one or more amino acids in the amino acid sequence of SEQ ID NO: 1, 3, 5 or 7,

(c) an amino acid sequence which has the voltage-gated potassium channel activity and which has sequence identity of 60% or more to the amino acid sequence of SEQ ID NO: 1, 3, 5 or 7,

(d) an amino acid sequence encoded by the nucleotide sequence of SEQ ID NO: 2, 4, 6 or 8,

(e) an amino acid sequence which has the voltage-gated potassium channel activity and which is encoded by a nucleotide sequence that has sequence identity of 75% or more to the nucleotide sequence of SEQ ID NO: 2, 4, 6 or 8,

(f) an amino acid sequence which has the voltage-gated potassium channel activity and which is encoded by a polynucleotide, wherein said polynucleotide hybridizes under a stringent condition to a polynucleotide comprising a nucleotide sequence complementary to the nucleotide sequence of SEQ ID NO: 2, 4, 6 or 8,

(g) an amino acid sequence of an insect ERG-type voltage-gated potassium channel,

(h) an amino acid sequence of a cotton aphid ERG-type voltage-gated potassium channel, and

(i) an amino acid sequence which has the voltage-gated potassium channel activity and which is encoded by a polynucleotide amplifiable by PCR employing a cDNA of cotton aphid as a template, and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton'aphid ERG-type voltage-gated potassium channel,

SEQ ID NO:11, 13 or 15 is a nucleotide sequence of PCR 5′ primer, and SEQ ID NO: 12 or 14 is a nucleotide sequence of PCR 3′ primer;

21. Use of a nematode expressing an insect voltage-gated potassium channel in a form functional as an ion channel, as a research tool for providing an indicator to evaluate pesticidal activity;
22. Use of a nematode expressing the voltage-gated potassium channel of item 20 in a form functional as an ion channel, as a research tool for providing an indicator to evaluate pesticidal activity;
23. Use of a cell expressing the voltage-gated potassium channel of item 20 in a form functional as an ion channel, as a research tool for providing an indicator to evaluate pesticidal activity;
24. A polynucleotide comprising a nucleotide sequence encoding an amino acid sequence of the voltage-gated potassium channel of item 20;
25. The polynucleotide according to item 24, wherein the polynucleotide comprising the nucleotide sequence of SEQ ID NO: 2, 4, 6 or 8;
26. A polynucleotide comprising a nucleotide sequence complementary to a nucleotide sequence of the polynucleotide of item 24 or 25;
27. A polynucleotide comprising a partial nucleotide sequence of the polynucleotide of item 24 or 25, or a nucleotide sequence complementary to the partial nucleotide sequence;
28. The polynucleotide according to item 27, wherein the polynucleotide comprising the nucleotide sequence any of SEQ ID NO: 11 to 15;
29. A method of obtaining a polynucleotide comprising a nucleotide sequence encoding an amino acid sequence of a voltage-gated potassium channel, which comprises: a step of amplifying a desired polynucleotide by polymerase chain reaction using the polynucleotide of item 27 or 28 as a primer, a step of identifying the amplified desired polynucleotide, and a step of recovering the identified polynucleotide;
30. A method of obtaining a polynucleotide comprising a nucleotide sequence encoding an amino acid sequence of a voltage-gated potassium channel, which comprises: a step of detecting a desired polynucleotide by hybridization using the polynucleotide of item 26, 27 or 28 as a probe, a step of identifying the detected desired polynucleotide, and a step of recovering the identified polynucleotide;
31. A cyclic polynucleotide comprising the polynucleotide of item 24 or 25 that is operably linked to a promoter expressible in a host organism or a host cell;
32. A circular polynucleotide comprising the polynucleotide of item 24 or 25 that is operably linked to a promoter from a nematode;
33. The circular polynucleotide according to item 32, wherein the promoter from a nematode is a promoter of a myo-2 gene;
34. The circular polynucleotide according to item 31, 32 or 33, wherein the circular polynucleotide has a replication origin for autonomous replication in a host cell;
35. A process for producing a circular polynucleotide, which comprises ligating the polynucleotide of item 24 or 25 to a vector;
36. A transformant in which the polynucleotide of item 24 or 25 has been introduced therein;
37. A cell transiently expressing an insect voltage-gated potassium channel, in which a transcription product of the polynucleotide of item 24 or 25 has been introduced therein;
38. The transfromant according to item 36, wherein the transformant is a transformed Escherichia coli;
39. The transformant according to item 36, wherein the transformant is a transformed nematode;
40. The transformant according to item 39, wherein the nematode is Caenorhabdtis elegans;
41. A process for producing a transformant, which comprises introducing the polynucleotide of item 24 or 25 into a host cell;
42. A process for producing a voltage-gated potassium channel, which comprises a step of culturing the transformant of item 36, 38, 39 or 40, and recovering the produced voltage-gated potassium channel;
43. Use of the voltage-gated potassium channel as defined in item 20, or the polynucleotide of any of items 24 to 28, as a research tool;
44. The use according to item 43, wherein the research tool is a research tool for screening a pesticidal agent;
45. A method of measuring an ability of a test substance to modulate the activity of an insect voltage-gated potassium channel, comprising:

(1) a first step of measuring the electrophysiological activity of a voltage-gated potassium channel of a cell that expresses a voltage-gated potassium channel selected from among the following group A in a form functional as an ion channel in a system in which the cell contact with a test substance, and

(2) a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the cell in a system containing no test substance:

### <Group A>

(a) a protein comprising the amino acid sequence of SEQ ID NO: 1, 3, 5 or 7, or a partial sequence thereof functioning as an ion channel,

(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,

(c) a protein having the voltage-gated potassium channel activity and comprising an amino acid sequence having 60% or more sequence identity with the amino acid sequence of SEQ ID NO: 1, 3, 5 or 7,

(d) a protein comprising an amino acid sequence encoded by the nucleotide sequence of SEQ ID NO: 2, 4, 6 or 8,

(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,

(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,

(g) a protein comprising an amino acid sequence of an insect ERG-type voltage-gated potassium channel,

(h) a protein comprising an amino acid sequence of a cotton aphid ERG-type voltage-gated potassium channel, and

(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and
SEQ ID NO: 12 or 14 is a nucleotide sequence of PCR 3′ primer;
   46. The method according to item 45, wherein the cell is an oocyte from Xenopus laevis;
   47. A method of measuring an ability of a test substance to modulate an activity of an insect voltage-gated potassium channel, comprising:

(1) a first step of measuring the electrophysiological activity of a voltage-gated potassium channel of a nematode that expresses a voltage-gated potassium channel selected from among the following group A in a form functional as an ion channel in a system in which the nematode contact with a test substance, and

(2) a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the nematode in a system containing no test substance:

### <Group A>

(a) a protein comprising the amino acid sequence of SEQ ID NO: 1, 3, 5 or 7, or a partial sequence thereof functioning as an ion channel,

(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,

(c) a protein having the voltage-gated potassium channel activity and comprising an amino acid sequence having 60% or more sequence identity with the amino acid sequence of SEQ ID NO: 1, 3, 5 or 7,

(d) a protein comprising an amino acid sequence encoded by the nucleotide sequence of SEQ ID NO: 2, 4, 6 or 8,

(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,

(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,

(g) a protein comprising an amino acid sequence of an insect ERG-type voltage-gated potassium channel,

(h) a protein comprising an amino acid sequence of a cotton aphid ERG-type voltage-gated potassium channel, and

(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and SEQ ID NO: 12 or 14 is a nucleotide sequence of PCR 3′ primer;

48. The method according to item 47, wherein the nematode is Caenorhabdtis elegans; and
49. A system which comprises: a means for inputting/accumulating/managing data information related to an ability to change an activity of a voltage-gated potassium channel derived from an insect having a test substance, a means for inquiring/retrieving the data information based on a desired condition, and a means for displaying/outputting the inquired/retrieved result, regarding the test substance.

### BRIEF DESCRIPTION OF THE DRAWINGS

FIG. 1 shows result of Example 2.

FIG. 2 shows result of Example 6.

FIG. 3 shows result of Example 6.

FIG. 4 shows result of Example 6.

FIG. 5 shows result of Example 6.

FIG. 6 shows result of Example 6.

FIG. 7 shows result of Example 6.

FIG. 8 shows result of Example 6.

FIG. 9 shows result of Example 7.

FIG. 10 shows result of Example 10.

FIG. 11 shows result of Example 10.

### MODES FOR CARRYING OUT THE INVENTION

The present invention will be explained in detail below.

In the present invention, the “pests” indicates small animals which cause harm or discomfort to life of the people by harming man and animals directly or by damaging crops. Examples thereof include arthropod such as insects, mites and ticks and Nematoda such as nematodes, and typical examples of which are as follows:

Hemiptera: Delphacidae such as Laodelphax striatellus, Nilaparvata lugens and Sogatella furcifera, Deltocephalidae such as Nephotettix cincticeps and Empoasca onukii, Aphididae such as Aphis gossypii and Myzus persicae, Pentatomidae, Aleyrodidae such as Trialeurodes vaporariorum, Bemisia tabaci and Bemisia argentifolli, Coccidae, Tingidae, Psyllidae, etc.

### Lepidoptera:

Pyralidae such as Chilo suppressalis, Cnaphalocrocis medinalis, Ostrinia nubilalis and Parapediasia teterrella, Noctuidae such as Spodoptera litura, Spodoptera exigua, Pseudaletia separata, Mamestra brassicae, Agrotis ipsilon, Trichoplusia spp., Heliothis spp., Helicoverpa spp. and Earias spp., Pieridae such as Pieris rapae crucivora, Tortricidae such as Adoxophyes orana fasciata, Grapholita molesta and Cydia pomonella, Carposimidae such as Carposina niponensis, Bucculatricidae such as Lyonetia clerkella, Gracillariidae such as Phyllonorycter ringoniella, Phyllocnistidae such as Phyllocnistis citrella, Yponomeutidae such as Plutella xylostella, Gelechiidae such as Pectinophora gossypiella, Arctiidae, Tineidae, etc.

### Diptera:

Culex such as Culex pipiens pallens, Cules tritaeniorhynchus and Culex quinquefasciatus, Aedes such as Aedes aegypti and Aedes albopictus, Anopheles such as Anophelinae sinensis, Chironomidae, Muscidae such as Musca domestica and Muscina stabulans, Calliphoridae, Sarcophagidae, Fannia canicularis, Anthomyiidae such as Delia Platura and Delia antigua, Trypetidae, Drosophilidae, Psychodidae, Simuliidae, Tabanidae, Stomoxyidae, Agromyzidae, etc.

### Coleoptera:

Diabrotica such as Diabrotica virgifera virgifera and Diabrotica undecimpunctata howardi, Scarabaeidae such as Anomala cuprea and Anomala rufocuprea, Curculionidae such as Sitophilus zeamais, Lissorphoptrus oryzophilus and Calosobruchys chinensis, Tenebrionidae such as Tenebrio molitor and Tribolium castaneum, Chrysomelidae such as Oulema oryzae, Aulacophora femoralis, Phyllotreta striolata and Leptinotarsa decemlineata, Anobiidae, Epilachna spp. such as Epilachna vigintioctopunctata, Lyctidae, Bostrychidae, Cerambycidae, Paederus fuiscipes, etc.

### Thysanoptera:

Thripidae such as Thrips spp. including Thrips palmi, Frankliniella spp. including Frankliniella occidentalis and Sciltothrips spp. including Sciltothrips dorsalis, Phlaeothripidae, etc.

### Hymenoptera:

### Tenthredimidae, Formicidae, Vespidae, etc.

### Dictyoptera:

### Blattidae, Blattellidae, etc.

### Orthoptera:

### Acrididae, Gryllotalpidae etc.

### Siphonaptera:

Pulex irritans, etc.

### Anoplura:

Pediculus humanus capitis, etc.

### Isoptera:

### Termitidae, etc.

### Acarina:

Tetranychidae such as Tetranychus urticae, Tetranychus kanzawai, Panonychus citri, Panonychus ulmi, and Oligonychus spp., Eriophyidae such as Aculops pelekassi and Aculus schlechtendali, Tarsonemidae such as Polyphagotarsonemus latus, Tenuipalpidae, Tuckerellidae, Ixodidae such as Haemaphysalis longicornis, Haemaphysalis flava, Dermacentor taiwanicus, Ixodes ovatus, Ixodes persulcatus and Boophilus microplus, Acaridae such as Tyrophagus putrescentiae, Dermanyssidae, Cheyletidae such as Dermatophagoides farinae and Dermatophagoides ptrenyssnus, such as Cheyletus eruditus, Cheyletus malaccensis and Cheyletus moorei, Dermanyssus spp., etc.

### Nematodes:

Pratylenchus coffeae, Pratylenchus fallax, Heterodera glycines, Globodera rostochiensis, Meloidogyne hapla, Meloidogyne incognita, etc.

In the present invention, the “modulate physiological condition of pests” indicates changing condition such as various phenomena in a living body which are maintained for living in pests, for example, function such as aspiration, digestion, secretion, body liquid circulation, metabolism, neurotransmission and the like, or mechanism thereof into condition apart from usual condition. Examples include changing condition by cessation of aspiration so that oxygen necessary for internal metabolism of pests is not supplied, and changing condition by cessation of function of neurotransmission of pests so that various movements of pests are ceased.

In the present invention, the “agent which modulates physiological condition of pests” is an agent which can modulate physiological condition of pests when being applied to the pests.

In the present invention, the “voltage-gated potassium channel from an insect” indicates a voltage-gated potassium channel exists in an insect, among voltage-gated potassium channels present in a variety of organisms. As the voltage-gated potassium channel, it is preferable that the voltage-gated potassium channel from an insect be an insect voltage-gated potassium channel from the EAG family, it is more preferable that the insect voltage-gated potassium channel from the EAG family be an insect ERG-type voltage-gated potassium channel (seizure), and it is further preferable that the insect ERG-type voltage-gated potassium channel be an ERG-type voltage-gated potassium channel (seizure) from a cotton aphid.

Insect is an animal classified under Animalia, Arthropoda, Insecta, and examples of which include arthropod of the order Protura, Collembola, Diplura, Thysanura, Ephemeroptera, Odonata, Plecoptera, Grylloblattodea, Orthoptera, Phasmatodea, Dermaptera, Mantodea, Blattaria, Isoptera, Embioptera, Psocoptera, Mallophaga, Anoplura, Thysanoptera, Hemiptera, Neuroptera, Mecoptera, Trichoptera, Lepidoptera, Coleoptera, Diptera, Hymenoptera, Siphonaptera, Strepsiptera, and the like.

The ion channel is one kind of transmembrane proteins present in a biological membrane of a cell, and makes ions permeate through a hydrophilic path called a pore formed at a center thereof. The ion channel is involved in maintenance of a membrane potential or a concentration of various ions inside and outside the cell, generation of an action potential in an electric excitable cell such as a nerve cell, and signal transduction.

An activity of the ion channel can be generally measured by an electrophysiological procedure. The activity of the voltage-gated potassium channel can be measured, for example, by a two-electrode voltage clamp method (TEVC method) using Xenopus laevis oocyte expressing an objective ion channel as described in a report of Bruggemann et al. (Nature 365 (6445), 445-448, 1993), or by a patch clamp method using a cultured cell expressing an objective ion channel as described in a report of Schonherr et al. (European Journal of Neuroscinece 11 (3), 753-760, 1999). In these methods, a flow of an ion is directly read using an electrode, and the activity of the ion channel is measured. In Caenorhabditis elegans (hereinafter, referred to as nematode in some cases) known as a model organism, a potential change accompanied with a constriction movement (pumping) of a pharynx can be measured using an electrophysiological procedure called electropharyngeogram, as described in a report of Raizena and Avery (Neuron 12 (3), 483-495, 1994). A waveform of electropharyngeogram reflects the activity of an ion channel and a transporter expressed at a site related to constriction of the pharynx such as the pharyngeal muscle. Therefore, by recording electropharyngeogram of a transgenic nematode expressing an objective ion channel at a pharynx, the activity of an objective ion channel can be indirectly measured as described later.

The method of measuring the activity of the insect voltage-gated potassium channel can be implemented by the similar method as the electrophysiological procedure.

In the present invention, “expressing an insect voltage-gated potassium channel in a form functional as an ion channel” means that the voltage-gated potassium channel is expressed in the state where the channel has an activity of making a potassium ion permeate, and that the activity can be measured by the aforementioned various electrophysiological procedures.

In the present invention, the “EAG family” consists of EAG (ether-a-go-go), ERG (EAG-related gene), and ELK (EAG-like gene), among the voltage-gated potassium channels. According to the classification name of the International Union of Basic and Clinical Pharmacology (IUPHAR), EAG is Kv10.x, ERG is Kvll.x, and ELK is Kv12.x. A human EAG family is also known as a KCNH family.

In the present invention, the “ERG-type voltage-gated potassium channel” refers to a product of a gene homologous to the ERG (EAG-related gene) which is a member of the EAG family. As an insect homolog of human ERG (hERG), seizure exists in Drosophila melanogaster. A seizure orthologue of a nematode (Caenorhabditis elegans) is unc-103.

Of the voltage-gated potassium channels from insects, as an amino acid sequence of the ERG-type voltage-gated potassium channel or seizure, amino acid sequences of:

Drosophila melanogaster (accession No. NP_{—}476713),
Tribolium castaneum (accession No. XP_{—}973853),
Apis mellifera (accession No. XP_{—}393977),
Anopheles gambiae (accession No. XP_{—}308166), and so on; have been disclosed in public databases.

Of the voltage-gated potassium channels from insects, as a nucleotide sequence encoding the ERG-type voltage-gated potassium channel or seizure, nucleotide sequences of:

Drosophila melanogaster (accession No. NM_{—}057365),
Tribolium castaneum (accession No. XM_{—}968760),
Apis mellifera (accession No. XM_{—}393977),
Anopheles gambiae (accession No. XM_{—}308166), and so on; have been disclosed in public databases.

By methods described later, it becomes possible to reveal an amino acid sequence of cotton aphid seizure and a nucleotide sequence of gene thereof which have not been known, and the amino acid sequences and the nucleotide sequences of genes obtained by these methods are disclosed in SEQ ID NO: 1, 3, 5, 7, and SEQ ID NO: 2, 4, 6, 8, respectively.

Identity of the amino acid sequence of cotton aphid seizure shown in SEQ ID NO: 3 with known sequences is shown in Table 1.

TABLE 1

| | Identity of amino |
|---|---|
| | acid sequence (%) |
| Anopheles gambiae | 80 |
| Drosophila melanogaster | 68 |
| Apis mellifera | 65 |
| Tribolium castaneum | 61 |
| Caenorhabditis elegans | 55 |
| Canis familiaris | 55 |
| Bos taurus | 54 |
| Gallus gallus | 53 |
| Mus musculus | 53 |
| Rattus norvegicus | 53 |
| Homo sapiens | 52 |
| Strongylocentrotus purpuratus | 51 |

In the present invention, the “ability to modulate an activity of an insect voltage-gated potassium channel” refers to an ability to increase or decrease the activity of an insect voltage-gated potassium channel, that is, an ability to activate, or an ability to inhibit the activity of a voltage-gated potassium channel. By adding a test substance to a reaction system for measuring the activity of a voltage-gated potassium channel using the electrophysiological procedure, an influence of the test substance on opening and closing of the voltage-gated potassium channel, and permeation of an ion can be checked.

For the ability to modulate the activity of the insect voltage-gated potassium channel, the electrophysiological activity of the voltage-gated potassium channel can be measured as follows using a cell expressing the voltage-gated potassium channel in a form functional as an ion channel.

For example, by introducing a gene of the insect voltage-gated potassium channel into an oocyte of Xenopus laevis to transiently express in the oocyte by the method described in Example 6, a cell expressing the voltage-gated potassium channel in a form functional as an ion channel can be prepared. Using the oocyte expressing the insect voltage-gated potassium channel, an electrophysiological activity of the voltage-gated potassium channel can be measured by a two-electrode voltage clamp method.

By introducing a gene of the insect voltage-gated potassium channel into a cultured animal cell to transiently express the gene in the cultured cell, a cell expressing the voltage-gated potassium channel in a form functional as an ion channel can be prepared. Alternatively, by introducing a gene of the insect voltage-gated potassium channel into a cultured animal cell, and selecting the cultured cell stably expressing the voltage-gated potassium channel from a transformed cultured cell population, a cell expressing the voltage-gated potassium channel in a form functional as an ion channel can be prepared. Herein, as the cultured animal cell, a CHO cell from a hamster which is a mammal, and a HEK293 cell from a human, a S2 cell from drosophila which is an insect, a Sf9 cell from an ovarian cell of Spodoptera frugiperda which is a Lepidoptera insect, and the like can be used. Using the cultured animal cell expressing the insect voltage-gated potassium channel, an electrophysiological activity of the voltage-gated potassium channel can be measured, for example, by a whole cell patch clamp method described in Example 7.

The ability to modulate the activity of the insect voltage-gated potassium channel can also be measured as follows using a transgenic nematode expressing the voltage-gated potassium channel in a form functional as an ion channel.

C. elegans known as a model organism transfers bacteria as food into the gut by a constriction movement (pumping) of the pharynx. The pharynx of C. elegans consists of such tissues as muscles and neurons, and is a validated model system for neuronal signaling and membrane excitability. For example, as described in a report of Davis et al. (Science 286 (5449), 2501-2504, 1999), the voltage-gated potassium channel EXP-2 in C. elegans influences the shape and duration of the action potential of pharyngeal muscle cells. In a loss-of-function mutant of EXP-2, repolarization was delayed in pharyngeal action potentials, and duration of the action potentials was prolonged. On the other hand, in a gain-of-function mutant of EXP-2, repolarization was accelerated, and duration of the action potentials was shortened.

Since the basic ion channels that control depolarization and repolarization of the action potential is conserved throughout the animal kingdom, it is possible to re-engineer the ion channel composition in the C. elegans pharynx, and develop a function model for an ion channel of interest. For example, when, of insect voltage-gated potassium channels, cotton aphid seizure is expressed in the C. elegans pharynx in a form functional as an ion channel, the presence of voltage-gated potassium channels in the pharynx is increased and hence the overall potassium efflux to the outside of a cell is increased. As a consequence, as described in Example 10 later, the repolarization of the pharynx action potential was accelerated, and duration of the action potential was shortened. This was the same as the phenotype of the gain-of-function mutant of EXP-2, and opposite to the phenotype of the loss-of-function mutant of EXP-2. This result shows that the genetically engineered pharynx is controlled by cotton aphid seizure.

It is possible to pharmacologically modulate the activity of the insect voltage-gated potassium channel which controls the action of the genetically engineered pharynx. For example, as shown in Example 6 and Example 7 described later, clofilium known as a blocker of human ERG or human EAG inhibited the activity of cotton aphid seizure. It was possible to measure the activity of cotton aphid seizure in the presence of clofilium by a conventional electrophysiological procedure. On the other hand, when C. elegans functionally expressing cotton aphid seizure in the pharynx is exposed to clofilium, as shown in Example 10, a shortened duration of action potential was prolonged again and the genetic-engineering induced phenotype was reversed to normal action potential duration, consistent with cotton aphid seizure inhibition in the electrophysiological experiment.

The alteration of the electrophysiological activity of the insect voltage-gated potassium channel which controls the genetically engineered pharynx influences the pharynx activity and hence the food intake. Therefore, the ability to modulate the activity of the insect voltage-gated potassium channel can be measured as the ability to modulate food intake of the transgenic nematode.

In the present invention, the “feeding behavior of a nematode” means an action of ingesting food by a constriction movement (pumping) of the pharynx in order to transfer bacteria as food into gut. Examples of a method of measuring the “feeding behavior of a nematode”, that is, the “food intake” include the following method using a fluorescent dye. When a pre-fluorescent compound is added to a medium containing a nematode, the pre-fluorescent compound orally ingested by the nematode is consequently converted enzymatically into a fluorescent dye in the gut, and a fluorescent signal emitted by the nematode is increased. Therefore, food intake by the nematode can be measured as a fluorescent signal emitted by the nematode. The principle assay is also called Drinking Assay as described in WO 00/63425, and has been validated with commercial compounds. For example, as described in WO 01/88532, the anthelmintic ivermectin inhibits food intake by a nematode. In the Drinking Assay this means that the intake of the pre-fluorescent dye is strongly inhibited and consequently the fluorescent signal is very low. Another example is the antidepressant clomipramine, which increases food intake by a nematode. In the Drinking Assay this means that the intake of the pre-fluorescent dye is strongly enhanced and consequently the fluorescent signal is very high. Applying this technology on a transgenic nematode that functionally expresses the insect voltage-gated potassium channel provides a means to check an influence of a test substance on the activity of the ion channel.

In addition, examples of a method for selecting a substance potentially having an ability to modulate the activity of an insect voltage-gated potassium channel include a binding assay using an RI-labeled ligand and a fluorescent dye method using a membrane potential-sensitive dye.

Among the aforementioned methods of measuring an ability to modulate the activity of the voltage-gated potassium channel, the Drinking Assay using the transgenic nematode is preferred for measuring the activity of the voltage-gated potassium channel in a large number of samples mechanically and efficiently. Examples thereof include a method of assessing an influence of a test substance on a feeding behavior activity as an influence on the activity of the ion channel. Specifically, a test substance is made to present in a liquid medium containing a nematode functionally expressing an insect voltage-gated potassium channel, and a pre-fluorescent dye is added to the medium. Intake of the pre-fluorescent dye orally ingested by the nematode is detected as a fluorescent signal emitted by the nematode. When intake of the pre-fluorescent dye is defined as the feeding behavior activity, an influence of a test substance on the feeding behavior activity is assessed as an influence on the activity of the ion channel.

Examples of the nematode functionally expressing an insect voltage-gated potassium channel include preferably a nematode functionally expressing a cotton aphid seizure, and examples of the pre-fluorescent dye include preferably calcein AM.

Among the various voltage-gated potassium channel blockers, clofilium (Gessner and Heinemann, Br. J. Pharmaco., 138: 161-171, 2003) has been known as a blocker of human ERG and human EAG, and is a model agent for treating arhythmia (Greene et al., Am. Heart J., 106: 492-501, 1983). While it has not been reported that clofilium acts on an insect voltage-gated potassium channel from the EAG family, clofilium inhibited the activity of cotton aphid seizure, as described in Example 6 and Example 7.

An EC₅₀ value of a test substance in the Drinking Assay means a concentration of a test substance at which the feeding behavior activity is reduced or increased by 50%, when intake of a pre-fluorescent dye by a nematode is defined as the feeding behavior activity. The EC₅₀ value of test substances can be determined by adding test substances at different concentrations to the Drinking Assay system, calculating the feeding behavior activity (response) of a nematode at each concentration of added test substance (dose), making a dose-response curve, and calculating a concentration of the added test substance at which the feeding behavior activity is reduced or increased by 50%. More specifically, using a 4 Parameter Logistic Model or a Sigmoidal Dose-Response Model:

fx=(A+(B−A)/(1+((C/x)̂D))),

$$f ( x ) = A + \frac{B - A}{1 + {( C / x )}^{D}}$$

a dose-response curve is made, thereby, EC₅₀ can be calculated. Practically, EC₅₀ can be calculated using XLfit (manufactured by IDBS) which is a commercially available calculation software.

The EC₅₀ value of a test substance in the electrophysiological procedure can also be calculated by the similar method, and means a concentration of the test substance at which the electrophysiological activity is reduced or increased by 50%. The EC₅₀ value of a test substance can be determined by adding test substances at different concentrations to the electrophysiological experimental system, calculating the electrophysiological activity (response) at each concentration of added test substance (dose), making a dose-response curve, and calculating a concentration of the added test substance at which the electrophysiological activity is reduced or increased by 50%.

In the present invention, the “agent that has an ability to modulate an activity of an insect voltage-gated potassium channel” is an agent containing, as an active ingredient, a substance having an ability to modulate the activity of the insect voltage-gated potassium channel.

In the present invention, the “agent that modulates physiological condition of pests, wherein the agent has an ability to modulate the activity of an insect voltage-gated potassium channel” means a agent whose ability to modulate the activity of an insect voltage-gated potassium channel can be identified by the measuring method, and which can modulate the physiological condition of a pest.

Examples of the agent include, preferably, the agent having an ability to modulate the activity of an insect voltage-gated potassium channel from the EAG family, more preferably, the agent having an ability to modulate the activity of an insect ERG-type voltage-gated potassium channel and, further preferably, the agent having an ability to modulate the activity of a cotton aphid ERG-type voltage-gated potassium channel.

In addition, examples of the agent preferably include a pesticidal agent.

In addition, examples of the agent preferably include the agent having an ability to modulate the electrophysiological activity of an insect voltage-gated potassium channel as measured by the above-mentioned electrophysiological procedure.

Alternatively, examples of the agent preferably include the agent having an ability to modulate the feeding behavior activity of the above-mentioned transgenic nematode in the Drinking Assay using the nematode.

In the present invention, the “pesticidal agent” indicates an agent having an ability to control the pests.

Examples of a method for measuring an ability to control pests include, in addition to the methods disclosed in the present invention, a method of measuring pesticidal activity on the pests. Specifically, for example, the pesticidal activity can be measured according to the following method.

According to the method described in Handbook of Insect Rearing Vol. 1 (Elsevier Science Publishers 1985), pp. 35 to pp. 36 except that a sterilized artificial feed having the following composition (Table 2) is prepared, and a solution of a test agent in DMSO is added at 0.5% by volume of the artificial feed and is mixed, a cotton aphid is reared, the number of surviving cotton aphids is investigated after 6 days, and a controlling value is obtained according to the following equation.

TABLE 2

| Amino acid | (mg/100 ml) | Vitamins | (mg/100 ml) |
|---|---|---|---|
| L-Alanine | 100.0 | Ascorbic acid | 100.0 |
| L-arginine | 275.0 | Biotin | 0.1 |
| L-Asparagine | 550.0 | Calcium | 5.0 |
| | | pantothenate | |
| L-Aspartic acid | 140.0 | Choline | 50.0 |
| | | chloride | |
| L-cysteine | 40.0 | Inositol | 50.0 |
| (hydrochloride) | | | |
| L-glutamic acid | 140.0 | Nicotinic acid | 10.0 |
| L-glutamine | 150.0 | Thiamine | 2.5 |
| L-glycine | 80.0 | | |
| L-histidine | 80.0 | Others | (mg/100 ml) |
| L-isoleucine | 80.0 | Sucrose | 12500.0 |
| L-leucine | 80.0 | Dipotassium | 1500.0 |
| | | hydrogen | |
| | | phosphate | |
| L-lysine | 120.0 | Magnesium | 123.0 |
| (hydrochloride) | | sulfate | |
| L-methionine | 80.0 | Cupric | 0.2 |
| | | chloride | |
| L-phenylalanine | 40.0 | Ferric | 11.0 |
| | | chloride | |
| L-proline | 80.0 | Manganese | 0.4 |
| | | chloride | |
| L-serine | 80.0 | Zinc sulfate | 0.8 |
| | | (anhydrous) | |
| L-threonine | 140.0 | | |

| | | |
|---|---|---|
| L-tryptophan | 80.0 | Adjusted to pH 6.8 |
| L-tyrosine | 40.0 | |
| L-valine | 80.0 | |

Controlling value(%)={1−(Cb×Tai)/(Cai×Tb)}×100

Letters in the equation represent the following meanings.

Cb: Number of surviving worms before treatment in non-treating section

Cai: Number of surviving worms at observation in non-treated section

Tb: Number of surviving worms before treatment in non-treated section

Tai: Number of surviving worms at observation in a treated section

It may be said that a test agent exhibiting a significantly high controlling value has the pesticidal activity. More preferably, it may be determined that a test agent having the controlling value of 30% or more has substantial pesticidal activity, and it may be determined that a test agent having the controlling value of less than 30% has no substantial pesticidal activity.

The pesticidal agent in the present invention contains a chemical substance having an ability to modulate the activity of insect voltage-gated potassium channel or an agriculturally acceptable salt thereof as an active ingredient.

In the present invention, an agriculturally acceptable salt refers to a salt in such a form that preparation of a controlling agent and application of the preparation do not become impossible, and may be a salt in any form. Specifically, examples of the salt include acid addition salts with mineral acids such as hydrochloric acid, hydrobromic acid, hydriodic acid, sulfuric acid, nitric acid, and phosphoric, organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethansulfonic acid, or acidic amino acids such as aspartic acid and glutamic acid; salts with inorganic bases such as sodium, potassium, magnesium, and aluminum, organic bases such as methylamine, ethylamine, and ethanolamine, or basic amino acids with lysine and ornithine; and an ammonium salts.

In the present invention, the “pesticidal agent which comprises a substance that has an ability to modulate the activity of an insect voltage-gated potassium channel or an agriculturally acceptable salt thereof as an active ingredient” means an agent which can control a pest by containing, as an active ingredient, a chemical substance whose ability to modulate the activity of the insect voltage-gated potassium channel can be identified by the measuring method, or an agriculturally acceptable salt thereof.

Examples of the agent preferably include the agent which comprises a substance having an ability to modulate the electrophysiological activity of the insect voltage-gated potassium channel as measured by the electrophysiological procedure.

Alternatively, examples of the agent preferably include the agent which comprises a substance having an ability to modulate a feeding behavior activity of the above-mentioned transgenic nematode in the Drinking Assay using the nematode.

Examples of the agent include the agent which comprises: a chemical substance having an ability to inhibit the feeding behavior activity of the above-mentioned transgenic nematode, wherein more preferably in the presence of the substance of 30 micro M or more the feeding behavior activity is lower than that in the absence of said substance in the Drinking Assay using the transgenic nematode; or a chemical substance having an ability to activate the feeding behavior activity of the above-mentioned transgenic nematode, wherein more preferably in the presence of said substance of 30 micro M or more the feeding behavior activity is higher than that in the absence of the substance in the Drinking Assay using the transgenic nematode.

In addition, examples of the agent further preferably include the agent which comprises: a chemical substance having an ability to inhibit the feeding behavior activity of the above-mentioned transgenic nematode, wherein an effective concentration of the substance at which the feeding behavior activity is reduced by 50% is 100 μM or lower in the Drinking Assay using the transgenic nematode; or a chemical substance having an ability to activate the feeding behavior activity of the above-mentioned transgenic nematode, wherein an effective concentration of the substance at which the feeding behavior activity is increased by 50% is 100 μM or lower in the Drinking Assay using the transgenic nematode.

In the present invention, the “method for assaying the pesticidal activity of a test substance, comprising: a first step of measuring the feeding behavior activity of a nematode that expresses a voltage-gated potassium channel selected from among the group A in a form functional as an ion channel in a system in which the nematode contacts with a test substance; and a second step of assessing the pesticidal activity of the test substance based on a difference obtained by comparing the feeding behavior activity measured in the first step with the feeding behavior activity of the nematode in a system containing no test substance” indicates a method characterized by comprising the first step and the second step in a variety of methods of testing the pesticidal ability of a test substance.

In addition, in the present invention, the “method for assaying the pesticidal activity of a test substance comprising: a first step of measuring the electrophysiological activity of a voltage-gated potassium channel of a cell that expresses a voltage-gated potassium channel selected from among the group A in a form functional as an ion channel in a system in which the cell contact with a test substance; and a second step of assessing the pesticidal activity of the test substance based on a difference obtained by comparing the electrophysiological activity of the voltage-gated potassium channel measured in the first step with the electrophysiological activity of the voltage-gated potassium channel of the cell in a system containing no test substance” indicates a method characterized by comprising the first step and the second step, in a variety of methods of testing the pesticidal ability of a test substance.

Herein, the group A indicates:

### <Group A>

(a) a protein comprising the amino acid sequence of SEQ ID NO: 1, 3, 5 or 7, or a partial sequence thereof functioning as an ion channel,

(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,

(c) a protein having the voltage-gated potassium channel activity and comprising an amino acid sequence having 60% or more sequence identity with the amino acid sequence of SEQ ID NO: 1, 3, 5 or 7,

(d) a protein comprising an amino acid sequence encoded by the nucleotide sequence of SEQ ID NO: 2, 4, 6 or 8,

(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,

(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,

(g) a protein comprising an amino acid sequence of an insect ERG-type voltage-gated potassium channel,

(h) a protein comprising an amino acid sequence of a cotton aphid ERG-type voltage-gated potassium channel, and

(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and

SEQ ID NO:12 or 14 is a nucleotide sequence of PCR 3′ primer.

The first step is a “step of adding a test substance to a reaction system for measuring the feeding behavior activity of a nematode that expresses the voltage-gated potassium channels in a form functional as an ion channel to bring the nematode into contact with the test substance, and measuring the feeding behavior activity of the nematode”, or a “step of adding a test substance to a reaction system for measuring the electrophysiological activity of a cell that expresses the voltage-gated potassium channels in a form functional as an ion channel to bring the cell into contact with the test substance, and measuring the electrophysiological activity of the cell”.

In addition, the second step is a step of comparing the activity at measurement of a test substance and the activity of a control, and assessing the pesticidal activity based on the difference.

Herein, the control means, for example, when a test substance dissolved in a solvent is added to the reaction system, a test section in which only a solvent same as that used to dissolve the test substance is added.

The nematode expressing the voltage-gated potassium channel in a form functional as an ion channel used in the method for assaying the pesticidal activity of a test substance comprising the first step and the second step is a nematode expressing a protein shown in the group A in a form functional as an ion channel.

In addition, the cell expressing the voltage-gated potassium channel in a form functional as an ion channel used in a method for assaying the pesticidal activity of a test substance comprising the first step and the second step is a cell expressing a protein shown in the group A in a form functional as an ion channel.

Among proteins of the group A, a difference which may be recognized between an amino acid sequence of the protein represented by (a) and amino acid sequences of proteins represented by (b), (c), (d), (e), (f), (g), (h) and (i) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing such deletion, substitution, addition or the like may be the number in such a range that an “feeding behavior activity of a nematode expressing a voltage-gated potassium channel in a form functional as an ion channel” or an “electrophysiological activity of a cell expressing a voltage-gated potassium channel in a form functional as an ion channel” can be found.

Examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine; (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method.

Herein, examples of a site-directed mutagenesis include a method utilizing amber mutation (gapped duplex method, Nucleic Acids Res., 12, 9441-9456 (1984)), a method by PCR using primers for mutation introduction, and the like.

Examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA [Pearson & Lipman, Proc. Natl. Acad. Sci. USA, 4, 2444-2448 (1988)], BLAST [Altschul et al., Journal of Molecular Biology, 215, 403-410 (1990)], CLUSTAL W [Thompson, Higgins&Gibson, Nucleic Acid Research, 22, 4673-4680 (1994a)] and the like. The program is generally available at the website (http://www.ddbj.nig.ac.jp) of DNA Data Bank of Japan [International DNA Data Bank managed in National Institute of Genetics, Center for Information Biology and DNA Data Bank of Japan; CIB/DDBJ]. Alternatively, sequence identity can be also obtained using a commercially available sequence analyzing software. Specifically, for example, sequence identity can be calculated by performing homology analysis using GENETYX-WIN Ver. 5 (manufactured by Software Development Co. Ltd.) by a Lipman-Pearson method [Lipman, D. J. and Pearson, W. R., Science, 227, 1435-1441, (1985)] and producing alignment.

When two optimally aligned amino acid sequences as described above have a difference in sequence as a result of conservative amino acid substitution, the “sequence similarity” is used in order to express the conservation of substituted amino acids. It may be said that the sequence similarity exists between sequence pairs which have differences in sequence resulting from conservative amino acid substitutions. This type of sequence similarity can be analyzed using programs such as FASTA above. Amino acids may be divided into four groups of hydrophobic amino acids, neutral amino acids, acidic amino acids and basic amino acids. The substitution of an amino acid by another amino acid of the same group is termed conservative amino acid substitution.

Group of hydrophobic amino acids includes alanine (A), valine (V), leucine (L), isoleucine (I), methionine (M), tryptophan (W), phenylalanine (F) and proline (P).

Group of neutral amino acids includes glycine (G), serine (S), threonine (T), cysteine (C), tyrosine (Y), asparagine (N) and glutamine (Q).

Group of acidic amino acids includes aspartic acid (D) and glutamic acid (E).

Group of basic amino acids includes lysine (K), histidine (H) and arginine (R).

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 65° C. (high stringent condition). Alternatively, both of a salt concentration and a temperature can be changed.

A protein described in (h) indicates a ERG-type voltage-gated potassium channel (seisure) exists in a cotton aphid among insect voltage-gated potassium channels, and includes a protein comprising the amino acid sequence described in (a).

While proteins of the group A include a protein described in (c) which comprises an amino acid sequence that has sequence identity of 60% or more to the amino acid sequence of SEQ ID NO: 1 and which has voltage-gated potassium channel activity, a protein having voltage-gated potassium channel activity and comprising an amino acid sequence that has sequence identity of 65, 70 or 75% or more to the amino acid sequence of SEQ ID NO: 1 may be preferably used, and a protein having voltage-gated potassium channel activity and comprising an amino acid sequence that has sequence identity of 80, 85, 90 or 95% or more to the amino acid sequence of SEQ ID NO: 1 may be highly preferred.

A substance having a pesticidal ability can be screened by using a method of assaying a pesticidal ability by measuring a pesticidal ability or controlling effect on the aforementioned pests.

Alternatively, a substance having a pesticidal ability can be also screened by the method of assaying a pesticidal ability using a voltage-gated potassium channel. Specifically, when it has been identified that a pesticidal ability of a test substance is a certain value or more, or a certain value or less using the method of assaying a pesticidal ability using a voltage-gated potassium channel, a substance having a pesticidal ability can be screened by selecting the substance.

Since a substance selected by the screening method has a pesticidal ability, it can be used as a pesticidal agent containing the substance or an agriculturally acceptable salt as an active ingredient.

Control of pests can be usually performed by application an effective amount of a pesticidal agent to a crop protected, a pest, or a habitat of a pest.

When a pesticidal agent is used for agriculture and forestry, its application amount is usually 0.1 to 1000 g in terms of an amount of a pesticidal agent per 1000 m². When a pesticidal agent is formulated into an emulsion, a water-dispersible powder, a flowable preparation, a microcapsule preparation or the like, the agent is usually applied by diluting with water to an active ingredient concentration of 1 to 10,000 ppm, and spraying this and, when a pesticidal agent is formulated into a granule, a powder or the like, the agent is usually applied as it is.

A pesticidal agent can be used by foliage-treating a plant such as a crop and the like which should be protected from pests, and can be also used by treating a seedbed before a plantlet of a crop is transplanted, or a planting hole or a strain base at planting. Further, for the purpose of controlling pests habiting a soil of a cultivating land, the agent may be used by treating the soil. Alternatively, the agent may be used by a method of winding a resin preparation which has been processed to a sheet or a string, on a crop, stretching the preparation near a crop and/or spreading on a soil surface of a strain base.

When a pesticidal agent is used as a pest controlling agent for preventing an epidemic, an emulsion, a water-dispersible powder, a flowable or the like is usually applied by diluting with water so that an active ingredient concentration becomes 0.01 to 10,000 ppm, and an oily agent, an aerosol, a fumigant, a poison bait or the like is applied as it is.

Examples of one utility of a pesticidal agent include control of an external parasite of a livestock such as cattle, sheep, goat, and chicken, or a small animal such as dog, cat, rat, and mouse, in this case, the agent can be administered to an animal by the veterinarily known method. As a specific administration method, when systemic control is intended, the agent is administered, for example, by a tablet, mixing in feed, suppository, injection (intramuscular, subcutaneous, intravenous, intraperitoneal etc.) and the like, when non-systemic control is intended, the agent is used by a method of spraying an oily agent or an aqueous liquid agent, performing pour on or spot on treatment, washing an animal with a shampoo preparation or attaching a resin preparation which has been processed into a necklace or a ear tag to an animal. An amount of a pesticidal agent when administered to an animal body is usually in a range of 0.1 to 1,000 mg as expressed by total amount of a compound A and a compound B per 1 kg of an animal.

An application amount and an application concentration of them are both different depending on the situations such as a kind of a preparation, an application time, an application place, an application method, a kind of a pest, a damage degree and the like, can be increased or decreased regardless of the aforementioned range, and can be appropriately selected.

The aforementioned pesticidal agent can be used in the method of controlling pests as described above.

In addition, a pest can be also controlled by identifying a substance having a pesticidal ability evaluated by the aforementioned method of assaying a pesticidal ability of a test substance comprising: “the first step and the second step using a nematode that expresses a voltage-gated potassium channel selected from among the group A in a form functional as an ion channel” or “the first step and the second step using a cell that expresses a voltage-gated potassium channel selected from among the group A in a form functional as an ion channel”; and bringing the identified substance having a pesticidal ability into contact with a pest. Herein, as a method of bringing an identified substance having a pesticidal ability into contact with a pest, the aforementioned preparation method, application method and the like can be used.

Amino acid sequences shown in the group B are amino acid sequences of insect voltage-gated potassium channels comprising any amino acid sequence of the following (a) to (i).

(a) the amino acid sequence of SEQ ID NO: 1, 3, 5 or 7,

(b) an amino acid sequence which has the voltage-gated potassium channel activity and which has deletion, addition or substitution of one or more amino acids in the amino acid sequence of SEQ ID NO: 1, 3, 5 or 7,

(c) an amino acid sequence which has the voltage-gated potassium channel activity and which has sequence identity of 60% or more to the amino acid sequence of SEQ ID NO: 1, 3, 5 or 7,

(d) an amino acid sequence encoded by the nucleotide sequence of SEQ ID NO: 2, 4, 6 or 8,

(e) an amino acid sequence which has the voltage-gated potassium channel activity and which is encoded by a nucleotide sequence that has sequence identity of 75% or more to the nucleotide sequence of SEQ ID NO: 2, 4, 6 or 8,

(f) an amino acid sequence which has the voltage-gated potassium channel activity and which is encoded by a polynucleotide, wherein said polynucleotide hybridizes under a stringent condition to a polynucleotide comprising a nucleotide sequence complementary to the nucleotide sequence of SEQ ID NO: 2, 4, 6 or 8,

(g) an amino acid sequence of an insect ERG-type voltage-gated potassium channel,

(h) an amino acid sequence of a cotton aphid ERG-type voltage-gated potassium channel, and

(i) an amino acid sequence which has the voltage-gated potassium channel activity and which is encoded by a polynucleotide amplifiable by PCR employing a cDNA of cotton aphid as a template, and using as primers a polynucleotide comprising the nucleotide sequence of SEQ ID NO: 11, 13 or 15, and a polynucleotide comprising the nucleotide sequence of SEQ ID NO: 12 or 14;

wherein:

SEQ ID NO:1, 3, 5 or 7 is an amino acid sequence of a cotton aphid ERG-type voltage-gated potassium channel,

SEQ ID NO:2, 4, 6 or 8 is a nucleotide sequence encoding a cotton aphid ERG-type voltage-gated potassium channel,

SEQ ID NO:11, 13 or 15 is a nucleotide sequence of PCR 5′ primer, and SEQ ID NO: 12 or 14 is a nucleotide sequence of PCR 3′ primer.

Among amino acid sequences of the group B, a difference which can be recognized between an amino acid sequence represented by (a) and amino acid sequences represented by (b), (c), (d), (e), (f), (g), (h) and (i) is deletion, substitution, addition or the like of a part of amino acids. These include, for example, deletion due to processing which the protein having an amino acid sequence represented by (a) undergoes in a cell. In addition, examples include deletion, substitution, addition and the like of an amino acid generated by naturally occurring gene mutation due to a spices difference or an individual difference of an organism from which the protein is derived, or gene mutation which is artificially introduced by a site-directed mutagenesis, a random mutagenesis, mutation treatment or the like.

The number of amino acids undergoing such deletion, substitution, addition or the like may be the number in such a range that an “feeding behavior activity of a nematode expressing a voltage-gated potassium channel in a form functional as an ion channel” or an “electrophysiological activity of a cell expressing a voltage-gated potassium channel in a form functional as an ion channel” can be found.

Examples of substitution of an amino acid include substitution with an amino acid which is similar in characteristic in hydrophobicity, charge, pH and steric structure. Specific examples of the substitution include substitution in an group of (1) glycine, alanine; (2) valine, isoleucine, leucine; (3) aspartic acid, glutamic acid, asparagine, glutamine, (4) serine, threonine; (5) lysine, arginine; (6) phenylalanine, tyrosine and the like.

Examples of a procedure of artificially introducing the deletion, addition or substitution of an amino acid (hereinafter, collectively referred to as alteration of amino acid in some cases) include a procedure of introducing site-directed mutation into a DNA encoding an amino acid sequence represented by (a) and, thereafter, expressing this DNA by a conventional method.

Herein, examples of a site-directed mutagenesis include a method utilizing amber mutation (gapped duplex method, Nucleic Acids Res., 12, 9441-9456 (1984)), a method by PCR using primers for mutation introduction, and the like.

Examples of a procedure of artificially altering an amino acid include a procedure of randomly introducing mutation into a DNA encoding an amino acid sequence represented by (a) and, thereafter, expressing this DNA by a conventional method. Herein, examples of a method of randomly introducing mutation include a method of performing PCR using a DNA encoding any of the aforementioned amino acid sequences as a template, and using a primer pair which can amplify each full length DNA at reaction condition under which an addition amount of each of dATP, dTTP, dGTP and dCTP used as a substrate is changed from a conventional concentration, or at reaction condition under which a concentration of Mg²⁺ promoting a polymerase reaction is increased from a conventional concentration. Examples of the procedure of PCR include a method described, for example, in Method in Molecular Biology, (31), 1994, 97-112. Another example includes a method described in WO 0009682.

Herein, the “sequence identity” refers to identity between two nucleotide sequences or two amino acids. The “sequence identity” is determined by comparing two sequences which are aligned in the optimal state over an all region of sequences to be compared. Herein, in optimal alignment of nucleotide sequences or amino acid sequences to be compared, addition or deletion (e.g. gap and the like) may be permitted. The sequence identity can be calculated by performing homology analysis to produce alignment using a program such as FASTA [Pearson & Lipman, Proc. Natl. Acad. Sci. USA, 4, 2444-2448 (1988)], BLAST [Altschul et al., Journal of Molecular Biology, 215, 403-410 (1990)], CLUSTAL W [Thompson, Higgins&Gibson, Nucleic Acid Research, 22, 4673-4680 (1994a)] and the like. The program is generally available at the website (http://www.ddbj.nig.ac.jp) of DNA Data Bank of Japan [International DNA Data Bank managed in National Institute of Genetics, Center for Information Biology and DNA Data Bank of Japan; CIB/DDBJ]. Alternatively, sequence identity can be also obtained using a commercially available sequence analyzing software. Specifically, for example, sequence identity can be calculated by performing homology analysis using GENETYX-WIN Ver. 5 (manufactured by Software Development Co. Ltd.) by a Lipman-Pearson method [Lipman, D. J. and Pearson, W. R., Science, 227, 1435-1441, (1985)] and producing alignment.

When two optimally aligned amino acid sequences as described above have a difference in sequence as a result of conservative amino acid substitution, the “sequence similarity” is used in order to express the conservation of substituted amino acids. It may be said that the sequence similarity exists between sequence pairs which have differences in sequence resulting from conservative amino acid substitutions. This type of sequence similarity can be analyzed using programs such as FASTA above. Amino acids may be divided into four groups of hydrophobic amino acids, neutral amino acids, acidic amino acids and basic amino acids. The substitution of an amino acid by another amino acid of the same group is termed conservative amino acid substitution.

Group of hydrophobic amino acids includes alanine (A), valine (V), leucine (L), isoleucine (I), methionine (M), tryptophan (W), phenylalanine (F) and proline (P).

Group of neutral amino acids includes glycine (G), serine (S), threonine (T), cysteine (C), tyrosine (Y), asparagine (N) and glutamine (Q).

Group of acidic amino acids includes aspartic acid (D) and glutamic acid (E).

Group of basic amino acids includes lysine (K), histidine (H) and arginine (R).

Examples of the “stringent condition” described in (f) include condition under which, in hybridization performed according to a conventional method described in Sambrook J., Frisch E. F., Maniatis T., Molecular Cloning 2nd edition, Cold Spring Harbor Laboratory press, for example, a hybrid is formed at 45° C. in a solution containing 6×SSC (a solution containing 1.5 m NaCl and 0.15 m trisodium citrate is 10×SSC) and, thereafter, this is washed with 2×SSC at 50° C. (Molecular Biology, John Wiley & Sons, N.Y. (1989), 6.3.1-6.3.6). A salt concentration in a washing step can be selected from condition from 2×SSC (low stringent condition) to 0.2×SSC (high stringent condition). A temperature in a washing step can be selected, for example, from condition from room temperature (low stringent condition) to 65° C. (high stringent condition). Alternatively, both of a salt concentration and a temperature can be changed.

The amino acid sequence described in (h) indicates an amino acid sequence of the ERG-type voltage-gated potassium channel (seisure) exists in a cotton aphid among insect voltage-gated potassium channels, and includes the amino acid sequence described in (a).

While proteins comprising amino acid sequences of the group B include a protein described in (c) which comprises an amino acid sequence that has sequence identity of 60% or more to the amino acid sequence of SEQ ID NO: 1 and which has voltage-gated potassium channel activity, a protein having voltage-gated potassium channel activity and comprising an amino acid sequence that has sequence identity of 65, 70, or 75% or more to the amino acid sequence of SEQ ID NO: 1 may be preferably used, and a protein having voltage-gated potassium channel activity and comprising an amino acid sequence that has sequence identity of 80, 85, 90 or 95% or more to the amino acid sequence of SEQ ID NO: 1 may be highly preferred.

A protein having an amino acid sequence shown in the group B can be prepared, for example, according to a method described later using a polynucleotide encoding an amino acid sequence shown in the group B.

The nematode expressing an insect voltage-gated potassium channel in a form functional as an ion channel can be used as a research tool which provides an indicator to evaluate pesticidal activity. Specifically, for example, the nematode expressing an insect voltage-gated potassium channel in a form functional as an ion channel can be used as a research tool which provides an indicator to evaluate pesticidal activity by being used as a nematode expressing a voltage-gated potassium channel in a form functional as an ion channel used in the method of assaying the pesticidal activity using the nematode expressing the voltage-gated potassium channel in a form functional as an ion channel. In addition, a more specific method can be implemented according to a method of measuring a feeding behavior activity of the nematode expressing a voltage-gated potassium channel in a form functional as an ion channel.

When the nematode expressing an insect voltage-gated potassium channel in a form functional as an ion channel is used as a research tool which provides an indicator to evaluate pesticidal activity, it is preferable that the insect voltage-gated potassium channel be a voltage-gated potassium channel comprising an amino acid sequence shown in the group B.

The cell expressing an insect voltage-gated potassium channel in a form functional as an ion channel can be used as a research tool which provides an indicator to evaluate pesticidal activity. Specifically, for example, the cell expressing an insect voltage-gated potassium channel in a form functional as an ion channel can be used as a research tool which provides an indicator to evaluate pesticidal activity by being used as a cell expressing a voltage-gated potassium channel in a form functional as an ion channel used in the method of assaying the pesticidal activity using the cell expressing the voltage-gated potassium channel in a form functional as an ion channel. In addition, a more specific method can be implemented according to an activity measuring method by an electrophysiological procedure using the cell expressing a voltage-gated potassium channel in a form functional as an ion channel.

When the cell expressing an insect voltage-gated potassium channel in a form functional as an ion channel is used as a research tool which provides an indicator to evaluate pesticidal activity, it is preferable that the insect voltage-gated potassium channel be a voltage-gated potassium channel comprising an amino acid sequence shown in the group B.

A polynucleotide comprising a nucleotide sequence encoding an amino acid sequence shown in the group B (hereinafter, referred to as polynucleotide group B in some cases) has a nucleotide sequence from which a protein comprising an amino acid sequence shown in the group B can be produced, in a cell of an organism or an in vitro translation system. A polynucleotide group B may be a DNA cloned from a nature, a DNA in which deletion, substitution or addition of a nucleotide is introduced into a DNA cloned from a nature, for example, by a site-directed mutagenesis or a random mutagenesis, or an artificially synthesized DNA. Specifically, examples include a polynucleotide comprising the nucleotide sequence of SEQ ID NO: 2, 4, 6 or 8.

### <First Obtaining Method>

For example, a method of obtaining a polynucleotide comprising the nucleotide sequence of SEQ ID NO: 2, 4, 6 or 8 included in the polynucleotide group B will be shown below. As a step, total RNA is obtained from cotton aphids, cDNA library is synthesized, and PCR amplification is performed, thereby, a polynucleotide of interest can be obtained.

A population of adults and larvae of Aphis gossypii, which have been reared on leaves of potted cucumber, is scraped from the surface of the leaves with a small brush, and 630 mg of the obtained population is crushed into a powder in liquid nitrogen using a mortar and a pestle. From the resulting frozen crushed powder, RNA is isolated using a RNA extracting reagent ISOGEN (manufactured by Nippon Gene) as follows. After 10 ml of ISOGEN is added to the frozen crushed powder in the mortar, the crushed powder is ground for 10 minutes while kept on ice. After grinding, a fluid sample is transferred to a 15 ml tube with a pipette, and 2 ml of chloroform (manufactured by Wako Pure Chemical Industries, Ltd.) is added thereto. Immediately, the mixture is vigorously shaken for 15 seconds and then left at rest at room temperature for 3 minutes. Then, the resulting mixture is centrifuged at 12,000×g at 4° C. for 15 minutes, and each 5 ml of aqueous layer are transferred to two new tubes. After 5 ml of ISOGEN is added to each tube, the mixture was immediately shaken vigorously for 15 seconds, and left at rest at room temperature for 3 minutes. Then, the resulting mixture is centrifuged at 12,000×g at 4° C. for 15 minutes, and each 10 ml of aqueous layer are transferred to new 50 ml tubes, respectively. Subsequently, 10 ml of isopropanol (manufactured by Wako Pure Chemical Industries, Ltd.) is added to each tube, and the mixture is kept on ice for 30 minutes. The resulting mixture is centrifuged at 12,000×g at 4° C. for 10 minutes to precipitate RNA. After the supernatant is removed, 20 ml of 70% ethanol is added to the residue. The resulting mixture is centrifuged at 10,000×g at 4° C. for 5 minutes. After the supernatant is removed, the precipitate of total RNA is slightly dried and then dissolved in 1 ml of commercially available RNase-free water (Nacalai Tesque, Inc.). An absorbance of the prepared total RNA is measured at 260 nm to calculate a concentration according to a conventional method.

RT-PCR is performed employing total RNA of cotton aphid obtained by the aforementioned method as a template, and using random primers (manufactured by Invitrogen) and superscript III (manufactured by Invitrogen) according to the manual annexed to the reagent, to synthesized a first-strand cDNA.

PCR is performed employing the cDNA of cotton aphid obtained by the aforementioned method as a template, and using an oligonucleotide primer comprising the nucleotide sequence of SEQ ID NO: 11 and an oligonucleotide primer comprising the nucleotide sequence of SEQ ID NO: 12 as well as a Expand Long Template PCR System (manufactured by Roche) according to the manual annexed to the reagent. The PCR conditions are those for touchdown PCR as follows: an initial denaturation at 94° C. for 2 minutes; followed by 10 cycles of touchdown-PCR, one cycle being 94° C. for 30 seconds, 70° C. for 30 seconds with a decrease of 1° C. per cycle, and 68° C. for 4 minutes; followed by 25 cycles of PCR, one cycle being 94° C. for 30 seconds, 60° C. for 30 seconds, and 68° C. for 30 seconds with a increase of 1° C. per cycle; and followed by 68° C. for 7 minutes.

As described above, a polynucleotide comprising the nucleotide sequence of SEQ ID NO: 2, 4, 6 or 8 can be obtained.

### <Second Obtaining Method>

Alternatively, a polynucleotide shown in the polynucleotide group B can be also obtained by preparing a polynucleotide with mutation introduced therein by a method utilizing amber mutation which is the aforementioned site-directed mutagenesis, a method by PCR using a primer for introducing mutation or the like, using as a template a polynucleotide comprising the nucleotide sequence of SEQ ID NO: 2, 4, 6 or 8.

### <Third Obtaining Method>

Alternatively, a polynucleotide shown in the polynucleotide group B can be also obtained by a hybridization method using a polynucleotide comprising the nucleotide sequence of SEQ ID NO: 2, 4, 6 or 8 as a probe. More specifically, the third obtaining method can be performed according to a conventional hybridization described in the aforementioned Sambrook J., Frisch E. F., Maniatis T., Molecular Cloning 2nd edition, published by Cold Spring Harbor Laboratory press.

### <Fourth Obtaining Method>

Alternatively, a polynucleotide shown in the polynucleotide group B can be also obtained by preparing a primer based on an amino acid sequence of the known insect voltage-gated potassium channel and performing PCR. For isolation of homologues of voltage-gated potassium channel gene from other insect species such as German cockroach (Blatella germanica), degenerate primers are designed using Codehop program (publicly accessible on the website of Blocks Protein Analysis Server operated within the Fred Hutchinson Cancer Research Center at http://blocks fhcrc.org/blocks/codehop.html), referring to the sequence of the aforementioned cotton aphid ERG-type voltage-gated potassium channel (seizure) gene and the previously-known amino acid sequences of homologues from human (NCBI accession number Q12809 and Q9H252), Caenorhabditis elegans (NP_{—}49782), Drosophila melanogaster (AAM68296), Anopheles gambiae (XP_{—}308166), and the like.

Partial sequences of a homologue of voltage-gated potassium channel gene of a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. The PCR conditions are those for touchdown PCR as follows: an initial denaturation at 94° C. for 5 minutes; followed by 10 cycles of touchdown-PCR, one cycle being 94° C. for 30 seconds, 60° C. for 1 minute with a decrease of 1° C. per cycle, and 72° C. for 1 minute and 30 seconds; followed by 25 cycles of PCR, one cycle being 94° C. for 30 seconds, 50° C. for 30 seconds, and 72° C. for 1 minute and 30 seconds; and followed by 72° C. for 7 minutes. The PCR product is analyzed and purified by agarose gel electrophoresis to obtain DNA of interest. Further, the obtained DNA is cloned into the pCR4-TOPO vector (manufactured by Invitrogen), and sequenced.

Then, primers specific for the resulting partial sequences of the insect homologue of voltage-gated potassium channel gene are designed, and 3′ RACE PCR or 5′ RACE PCR is performed in order to obtain a full-length sequence of the gene.

In 5′ RACE reaction, a reverse primer specific for the sequence of interest is used in combination with Oligo-d (T)-anchor primer1 contained in 5′/3′ RACE Kit, 2nd Generation as forward primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 40 seconds; followed by 25 cycles of PCR, one cycle being 95° C. for 30 seconds, 50° C. for 30 seconds, and 72° C. for 40 seconds extended with 20 seconds each cycle; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

When a distinct amplification product is not obtained by the first-round PCR, nested PCR is performed using the first-round PCR product as a template. As primers, a specific reverse primer which is designed to bind to internal sequence of the first-round PCR product is used in combination with PCR Anchor primer contained in 5′/3′ RACE Kit, 2nd Generation as a forward primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 40 seconds; followed by 25 cycles of PCR, one cycle being 95° C. for 30 seconds, 50° C. for 30 seconds, and 72° C. for 40 seconds extended with 20 seconds each cycle; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

In 3′ RACE reaction, a forward primer specific for the sequence of interest is used in combination with universal primer mix (UPM) contained in SMART PCR cDNA Synthesis Kit as a reverse primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 1 minute; followed by 25 cycles of PCR, one cycle being 95° C. for 30 seconds, 50° C. for 30 seconds, and 72° C. for 1 minute; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

When a distinct amplification product is not obtained by the first-round PCR, nested PCR is performed using the first-round PCR product as a template. As primers, a specific forward primer which is designed to bind to internal sequence of the first-round PCR product is used in combination with NUP primer contained in SMART PCR cDNA Synthesis Kit as a reverse primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 1 minute; followed by 25 cycles of PCR, one cycle being 95° C. for 30 seconds, 50° C. for 30 seconds, and 72° C. for 1 minute; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

The above sequencing results reveal 5′-terminal sequence and 3′-terminal sequence, each encoding N-terminal region and C-terminal region of the insect voltage-gated potassium channel, respectively.

Thus, a polynucleotide shown in the polynucleotide group B can be obtained by PCR by preparing a primer based on an amino acid sequence of the known insect voltage-gated potassium channel.

A polynucleotide comprising a nucleotide sequence complementary to a nucleotide sequence of the polynucleotide group B can be used for obtaining a polynucleotide shown in the polynucleotide group B using a hybridization method.

The obtaining method in the present invention comprises a step of detecting a desired polynucleotide by hybridization, a step of identifying the detected desired polynucleotide, and a step of recovering the identified polynucleotide. Each step will be explained specifically below.

A step of detecting a desired polynucleotide by hybridization, and a step of identifying the detected desired polynucleotide can be performed by using, as a probe, a polynucleotide having a nucleotide sequence having complementarity to a nucleotide sequence of a polynucleotide group B, according to the method described, for example, in “Molecular Cloning: A Laboratory Manual 2nd edition” (1989), Cold Spring Harbor Laboratory Press, “Current Protocols In Molecular Biology” (1987), John Wiley & Sons, Inc. ISBN0-471-50338-X and the like.

Specifically, for example, a DNA comprising a nucleotide sequence complementary to the nucleotide sequence of SEQ ID NO: 2 is labeled with a radioisotope or a fluorescently labeled by the known method using Random Primed DNA Labelling Kit (manufactured by Boehringer), Random Primer DNA Labelling Kit Ver. 2 (manufactured by TAKARA SHUZO Co., Ltd.), ECL Direct Nucleic Acid Labelling and Ditection System (manufactured by Amersham Biosciences), or Megaprime DNA-labelling system (manufactured by Amersham Biosciences), and this can be used as probe.

Examples of condition for hybridization include stringent condition, and specifically, examples include condition under which incubation is performed at 65° C. in the presence of 6×SSC (0.9M NaCl, 0.09M sodium citrate), a 5×Denhart's solution (0.1% (w/v) Ficoll 400, 0.1% (w/v) polyvinylpyrrolidone, 0.1% BSA), 0.5% (w/v) SDS and 100 μg/ml denatured salmon spermatozoon DNA, or in a DIG EASY Hby solution (Boehringer Mamnnheim) containing 100 μg/ml denatured salmon spermatozoon DNA, then, incubation is performed two times at room temperature for 15 minutes in the presence of 1×SSC (0.15 m NaCl, 0.015 m sodium citrate) and 0.5% SDS and, further, incubation is performed at 68° C. for 30 minutes in the presence of 0.1×SSC (0.015 m NaCl, 0.0015 m sodium citrate) and 0.5% SDS.

More specifically, for example, a probe labeled with ³²P can be made by employing a polynucleotide comprising a nucleotide, sequence complementary to a nucleotide sequence of a polynucleotide group B as a template, using Megaprime DNA-labelling system (manufactured by Amersham Pharmacia Biotech) and using a reaction solution designated in a kit. Colony hybridization is performed using this probe according to a conventional method, incubation is performed at 65° C. in the presence of 6×SSC (0.9M NaCl, 0.09M sodium citrate), a 5×Denhart's solution (0.1% (w/v) Ficoll 400, 0.1% (w/v) polyvinylpyrrolidone, 0.1% BSA), 0.5% (w/v) SDS and 100 μg/ml denatured salmon spermatozoon DNA, or in a DIG EASY Hyb solution (Boehringer Mannheim) containing 100 μg/ml denatured salmon spermatozoon DNA, then, incubation is performed two times at room temperature for 15 minutes in the presence of 1×SSC (0.15 m NaCl, 0.015 m sodium citrate) and 0.5% SDS and, further, incubation is performed at 68° C. for 30 minutes in the presence of 0.1×SSC (0.015 m NaCl, 0.0015 m sodium citrate) and 0.5% SDS, thereby, (a colony containing) a hybridizing polynucleotide can be detected. Thus, a desired polynucleotide can be detected by hybridization, and the detected desired polynucleotide can be identified.

For recovering the identified polynucleotide, a plasmid DNA can be recovered from a colony containing the polynucleotide detected and identified by the aforementioned method, for example, according to a method such as the alkali method described in “Molecular Cloning: A Laboratory Manual 2nd edition” (1989), Cold Spring Harbor Laboratory Press. A nucleotide sequence of the recovered desired polynucleotide (plasmid DNA) can be confirmed by a Maxam Gilbert method (described, for example, in Maxam, A. M & W. Gilbert, Proc. Natl. Acad. Sci. USA, 74, 560, 1977 etc.) or a Sanger method (described, for example, in Sanger, F. & A. R. Coulson, J. Mol. Biol., 94, 441, 1975, Sanger, F, & Nicklen and A. R. Coulson., Proc. Natl. Acad. Sci. USA, 74, 5463, 1977 etc.). Thereupon, for example, commercially available Termo Segenase II dye terminator cycle sequencing kit (manufactured by Amersham biosciences), Dye Terminator Cycle Sequencing FS Ready Reaction Kit (manufactured by Applied Biosystems) and the like can be used.

A polynucleotide comprising a partial nucleotide sequence of a nucleotide sequence of the polynucleotide group B or a nucleotide sequence complementary to the partial nucleotide sequence can be used for obtaining a polynucleotide shown in the polynucleotide group B using PCR. More specifically, examples include a polynucleotide comprising a nucleotide sequence of any of SEQ ID NOs: 11 to 15. The obtaining method in the present invention includes a step of amplifying a desired polynucleotide by PCR, a step of identifying the amplified desired polynucleotide, and a step of recovering the identified desired polynucleotide. Each step will be specifically explained below.

In a step of amplifying a desired polynucleotide by PCR, specifically, a DNA designed and synthesized from a partial nucleotide sequence of a nucleotide sequence of a polynucleotide group B or a nucleotide sequence complementary to the partial nucleotide sequence, based on an about 20 bp to about 40 bp nucleotide sequence, for example, a nucleotide sequence selected from a nucleotide sequence of SEQ ID NO: 2, 4, 6 or 8 and a sequence complementary to the nucleotide sequence of SEQ ID NO: 2, 4, 6 or 8 can be used as a primer set. Examples of a primer set include a set of a polynucleotide comprising a nucleotide sequence represented by SEQ ID NO: 13 and a polynucleotide comprising the nucleotide sequence of SEQ ID NO: 14. A PCR reaction solution is prepared, for example, by adding a reaction solution designated by a commercially available PCR kit to a cDNA library prepared by the aforementioned method. Reaction condition can be changed depending on a primer set to be used, and for example, condition under which after incubation at 94° C. for 10 seconds, around 40 cycles is repeated, 1 cycle being 94° C. for 15 seconds, 60° C. for 15 seconds, and 72° C. for 3 minutes and, further, incubation is performed at 72° C. for 3 minutes, condition under which incubation is performed at 94° C. for 2 minutes, thereafter, incubation is performed at about 8° C. for 3 minutes and, thereafter, around 40 cycles is repeated, 1 cycle being 94° C. for 30 seconds, 55° C. for 30 seconds, and 72° C. for 4 minutes, or condition under which 5 to 10 cycles is performed, 1 cycle being incubation at 94° C. for 5 seconds and, then, 72° C. for 4 minutes and, further, around 20 to 40 cycles is performed, 1 cycle being incubation at 94° C. for 5 seconds and, then, 70° C. for 4 minutes, can be used. In the PCR, for example, PfuUltra High Fidelity polymerase (manufactured by Stratagene), Amplitaq Gold (manufactured by Applied Biosystems), Takara Heraculase (Trademark) (manufactured by TAKARA SHUZO Co., Ltd.), a DNA polymerase contained in Advantage cDNA PCR Kit (manufactured by Clonetech), TaKaRa Ex Taq (manufactured by TAKARA SHUZO Co., Ltd.), PLATINUM™ PCR SUPER Mix (manufactured by Lifetech Oriental) can be used.

Identification of a desired polynucleotide amplified by PCR can be performed by measuring a molecular weight by agarose gel electrophoresis according to the method described in “Molecular Cloning: A Laboratory Manual 2nd edition” (1989), Cold Spring Harbor Laboratory Press. In addition, regarding the amplified desired polynucleotide, a sequencing reaction is performed using a commercially available DNA sequencing reaction kit, for example, Dye Terminator Cycle Sequencing FS Ready Reaction Kit (manufactured by Applied Biosystems) according to a manual annexed to the kit, and the nucleotide is analyzed using a DNA sequencer 3100 (manufactured by Applied Biosystems), thereby, a nucleotide sequence of the amplification fragment can be read.

Examples of a method of recovering the identified desired polynucleotide include a method of purifying and recovering the aforementioned polynucleotide identified by agarose gel electrophoresis from an agarose gel according to the method described in “Molecular Cloning: A Laboratory Manual 2nd edition” (1989), Cold Spring Harbor Laboratory Press. In addition, the thus recovered polynucleotide or a desired polynucleotide amplified by PCR can be cloned into a vector according to a conventional method described in “Molecular Cloning: A Laboratory Manual 2nd edition” (1989), Cold Spring Harbor Laboratory Press, and “Current Protocols In Molecular Biology” (1987), John Wiley & Sons, Inc. ISBN0-471-50338-X. Examples of a vector to be used include pUCA119 (manufactured by TAKARA SHUZO Co., Ltd.), pTVA118N (manufactured by TAKARA SHUZO Co., Ltd.), pBluescriptII (manufactured by Toyobo Co., Ltd.), pCR2.1-TOPO (manufactured by Invitrogen) and the like. In addition, a nucleotide sequence of the cloned polynucleotide can be confirmed by a Maxam Gilbert method (described, for example, in Maxam, A. M & W. Gilbert, Proc. Natl. Acad. Sci. USA, 74, 560, 1977) or a Sanger method (described, for example, in Sanger, F. & A. R. Coulson, J. Mol. Biol., 94, 441, 1975, Sanger, F, & Nicklen and A. R. Coulson., Proc. Natl. Acad. Sci. USA, 74, 5463, 1977). Thereupon, for example, a commercially available Termo Seqenase II dye terminator cycle sequencing kit (manufactured by Amersham biosciences), Dye Terminator Cycle Sequencing FS Ready Reaction Kit (manufactured by Applied Biosystems) and the like can be used.

In addition, a polynucleotide having a partial nucleotide sequence of a nucleotide sequence of the polynucleotide group B or a nucleotide sequence complementary to the partial nucleotide sequence can be used for obtaining a polynucleotide shown in the polynucleotide group B using not only a PCR method, but also the aforementioned hybridization method. More specifically, examples include a polynucleotide comprising a nucleotide sequence of any of SEQ ID NOs: 11 to 15.

Examples of a method for preparing a protein comprising an amino acid sequence shown in the group B include a method of culturing a transformant with a polynucleotide selected from a polynucleotide group B introduced therein, and recovering the produced protein. In addition, for preparing a transformant used herein, it is a work such as preparation of a circular polynucleotide containing a polynucleotide in which a polynucleotide selected from a polynucleotide group B is operably ligated to a promoter expressible in a host organism or in a host cell. The method will be explained in detail below.

In addition, a voltage-gated potassium channel shown in a group A which is used in the method of assaying a pesticidal activity using a voltage-gated potassium channel can be prepared and obtained by the similar method, using a polynucleotide comprising a nucleotide sequence encoding a voltage-gated potassium channel used.

The promoter expressible in a host organism or a host cell means a promoter having an ability to transcribe an objective gene to express the gene product in a transcription system of the host organism or the host cell into which a polynucleotide containing the objective gene has been introduced. For example, when a host is Escherichia coli, examples of the promoter include promoters of T7 RNA polymerase gene, T3 RNA polymerase gene, and SP6 RNA polymerase gene which are promoters from bacteriophage. When a host is a mammal cell, examples of the promoter include a CMV promoter which is a promoter of an IE (immediate early) gene derived from cytomegalovirus. When a host is a nematode, examples of the promoter include a promoter of a myosin gene (myo-2 or myo-3) derived from a nematode. In a nematode, a promoter of a myo-2 gene exhibits pharynx-specific expression, and a promoter of a myo-3 gene exhibits body wall-specific expression.

In the present invention, “operably linked” means that a polynucleotide containing a gene of interest is linked downstream of a polynucleotide containing a promoter sequence so that the gene of interest can be transcribed in a used transcription system. Specifically, for example, when CMV promoter is used, a polynucleotide containing a gene of interest may be linked downstream of CMV promoter. In addition, for example, when a promoter other than CMV promoter is used, it is also possible to link a polynucleotide containing a gene of interest downstream of a polynucleotide containing a promoter sequence other than CMV promoter. More specifically, for example, when a plasmid pcDNA3.1(+) (manufactured by Invitrogen) vector utilizing the CMV promoter is used, the polynucleotide can be operably linked by ligating a gene of interest into a restriction enzyme site such as NheI, PmeI, AflII, HindIII, Asp718I, KpnI, BamHI, BstXI, EcoRI, EcoRV, NotI, XhoI, XbaI, DraII, or ApaI located downstream of the CMV promoter.

In the present invention, the “circular polynucleotide” is a polynucleotide which has been made to be circular by binding of ends of the polynucleotide strand, and examples include chromosomal DNAs of many bacteria in addition to a plasmid DNA, a bacmid DNA and the like.

A plasmid DNA is a relatively low-molecular circular polynucleotide, and examples include pET (manufactured by Novagen) and pBluescriptII (manufactured by Stratagene), used for cloning and expression in E. coli. Other examples thereof include pcDNA (manufactured by Invitrogen) used in gene expression in a mammal cell.

A circular polynucleotide in which a polynucleotide comprising a nucleotide sequence encoding an amino acid sequence shown in the group B is operably linked to a promoter expressible in a host organism or a host cell is specifically, for example, a circular polynucleotide containing a DNA comprising a cotton aphid seizure gene operably linked to a bacteriophage T7 RNA polymerase promoter, cytomegalovirus CMV promoter or cotton aphid myo-2 gene promoter and can be prepared and obtained, for example, according to the following method.

DNA fragment containing the cotton aphid seizure gene is amplified by PCR, using a plasmid DNA containing a cotton aphid seizure gene cloned in accordance with the aforementioned method as a template, with a primer specific to the cotton aphid seizure gene to which a Eco RV restriction site is added and a primer specific to the cotton aphid seizure gene to which a XhoI restriction site is added. The resulting PCR products are cleaved with Eco RV and XhoI, and the obtained DNA fragment containing the cotton aphid seizure gene is ligated to a plasmid vector pcDNA3+) (manufactured by Invitrogen) digested in advance with Eco RV and XhoI. The plasmid obtained in this way is one example of circular polynucleotide containing DNA fragment comprising the cotton aphid seizure gene operably linked to CMV promoter.

Similarly, a circular polynucleotide can be prepared by ligating nucleotides encoding an amino acid sequence shown in the group B to a vector.

In the present invention, the “replication origin” is the specific DNA sequence necessary for replicating itself in a host cell. Examples of origin of replication include colE1, f1 and pUC for bacterial plasmids.

A transformant is a eukaryote (eukaryotic cell) or a prokaryote (prokaryotic cell) which has been genetically altered by introducing a foreign polynucleotide into a cell. Examples of the transformant include an Escherichia coli cell transformed by introducing a plasmid such as pET (manufactured by Novagen) or pBluescript II (manufactured by Stratagene) used in gene cloning or gene expression in Escherichia coli. Other examples thereof include a mammalian cultured cell such as a CHO cell or a HEK293 cell transformed by introducing a plasmid such as pcDNA (manufactured by Invitrogen) used in gene expression in a mammalian cell. Alternatively, examples thereof include an insect cultured cell such as a S2 cell derived from drosophila or a Sf9 cell derived from an ovarian cell of Spodoptera frugiperda which is a Lepidoptera insect, transformed by introducing a plasmid such as pMT (manufactured by Invitrogen) or pAC (manufactured by Invitrogen) used in gene expression in an insect cell. In a nematode of a model organism, examples thereof include a nematode transformed by introducing a plasmid in which an objective gene is connected to a promoter derived from a nematode.

Examples of the transformant in which a polynucleotide encoding an amino acid sequence shown in the group B has been introduced include transformed Escherichia coli in which a DNA fragment containing a cotton aphid seizure gene operably linked to a promoter of bacteriophage T7 RNA polymerase gene has been introduced, a transformed mammal cell in which a DNA fragment containing acotton aphid seizure gene operably linked to a CMV promoter of cytomegalovirus has been introduced, a transformed nematode in which a DNA fragment containing a cotton aphid seizure gene operably linked to a promoter of a myo-2 gene of a nematode has been introduced, and the like.

Examples of the technique of introducing a DNA into a host organism or a host cell include transformation, transfection, protoplast fusion, lipofection, electroporation, microinjection, and particle gun. In a nematode, a transformant is produced by introducing a foreign gene mainly by microinjection as described in a report of Mello et al. (EMBO J. 10 (12), 3959-3970, 1991) and a literature of Mello and Fire (Methods in Cell Biology 48, 451-482), and a transformation method by a particle gun is also developed as described in a report of Praitis et al. (Genetics 157, 1217-1226, 2001) and WO 99/49066.

A transformant of a nematode is distinguished by a phenotype of a marker gene which is simultaneously microinjected with an objective gene. The marker gene is classified into two kinds. One confers change to a phenotype which is easily distinguished such as a movement or morphology of a nematode. For example, there is a mutant gene which confers a new phenotype to a nematode such as rol-6 which confers a clockwise roller (Rol) phenotype, and a wild-type gene which rescues a phenotype of a mutation in a host such as dpy-20, lin-15, unc-76, unc-4, pha-1, and unc-119. The other is a fluorescent reporter gene in which a gene of a fluorescent protein such as a green fluorescent protein (GFP) is connected downstream of a promoter from a nematode.

Examples of selection of a transformant by rescue of a mutant using pha-1 as a selection marker include a method using a pBX vector described in a report of Granato et al. (Nucleic Acids Res. 22, 1762-1763, 1994). While the pha-1 mutant cannot be grown at 20° C. or higher, a transformant of the mutant in which a wild-type gene used as a marker has been introduced can be grown also at 20° C. or higher, due to rescue of the phenotype.

Examples of selection of a transformant by rescue of a mutant using unc-119 as a selection marker include a method using a pDPMM #016b vector described in a report of Praitis et al. (Genetics 157, 1217-1226, 2001). A nematode, when an individual density is high and a feed is lacked in a L1 larva phase, becomes a resistant larva and can survive for around 6 months with no feed. Since an unc-119 mutant cannot become the resistant larva, it cannot survive in the fasting state. A transformant of the mutant in which a wild-type gene used as a marker has been introduced can survive even in the fasting state, due to rescue of the phenotype.

A transformant in which a polynucleotide encoding an amino acid sequence shown in the group B has been introduced can be specifically produced according to the following method.

A transformant can be prepared by introducing into an Escherichia coli cell a plasmid vector pET41a(+) (Novagen) in which a DNA containing a cotton aphid voltage-gated potassium channel gene is inserted between Bam HI site and Xho I site, according to the method described in “Molecular Cloning: A Laboratory Manual 2nd edition” (1989), Cold Spring Harbor Laboratory Press. Alternatively, a transformant can be also prepared by transforming E. coli using the aforementioned plasmid DNA into which a fragment containing a bacteriophage T7 RNA polymerase promoter and a cotton aphid voltage-gated potassium channel gene is inserted, according to a method described in a manual annexed to Escherichia coli BL21 (DE3) competent cells (Invitrogen).

Transformed Escherichia coli can be produced by introducing into an Escherichia coli cell a plasmid vector pET (manufactured by Novagen) or pBluescript II (manufactured by Stratagene) in which a DNA fragment containing a cotton aphid seizure gene has been inserted, according to the method described in “Molecular Cloning: A Laboratory Manual 2^{nd} edition” (1989), Cold Spring Harbor Laboratory Press. Alternatively, transformed Escherichia coli can be obtained by transforming Escherichia coli using a plasmid DNA in which has been inserted the DNA fragment containing a cotton aphid seizure gene operably linked to the bacteriophage T7 RNA polymerase gene promoter, according to the method described in instructions attached to a competent cell of Escherichia coli DH5α or BL21 (DE3) (manufactured by Invitrogen).

A transformed cell can be obtained by introduction into a mammalian cell such as a CHO cell or a HEK293 cell using a plasmid DNA in which has been inserted the DNA fragment containing a cotton aphid seizure gene operably linked to the cytomegalovirus CMV promoter, according to the method described in instructions attached to lipofectamine (manufactured by Invitrogen), lipofectin (manufactured by Invitrogen), cellfectin (manufactured by Invitrogen) or the like.

In addition, a transformed nematode can be produced by introducing a foreign gene by the microinjection or the particle gun using a plasmid DNA in which has been inserted the DNA fragment containing a cotton aphid seizure gene operably linked to the nematode myo-2 gene promoter.

As a nematode expressing the voltage-gated potassium channel in a form functional as an ion channel or a cell expressing the voltage-gated potassium channel in a form functional as an ion channel, which are used in the method for assaying the pesticidal activity, a nematode or a cell expressing a protein shown in the group A can be produced and obtained by the similar method using a polynucleotide comprising a nucleotide sequence encoding the voltage-gated potassium channel used.

A voltage-gated potassium channel can be prepared by culturing a transformant prepared by the aforementioned method and recovering the produced insect voltage-gated potassium channel.

Examples of a transient expression cell in which a transcription product of a polynucleotide encoding an amino acid sequence shown in the group B has been introduced include an oocyte of Xenopus laevis to which a cRNA of a cotton aphid seizure gene has been introduced therein.

Examples of the technique of introducing an RNA into a host cell include mainly microinjection. The transient expression cell in which a transcription product of a polynucleotide encoding an amino acid sequence shown in the group B has been introduced can be specifically produced according to the following method.

Female Xenopus laevis is anesthetized, a lower abdominal part is cut small while ice-cooling, and an ovary containing an oocyte is taken out. A part of ovarian follicle is cut out, washed with an OR2 solution (82.5 mM NaCl, 2 mM KCl, 1 mM MgCl₂, 5 mM HEPES, pH 7.4) containing no calcium ion, and subjected to 2 mg/ml collagenase treatment at 16° C., and a follicle cell is removed from a surface of an oocyte. A plasmid containing a cotton aphid seizure gene operably linked to bacteriophage T7 RNA polymerase gene promoter is digested with a restriction enzyme for linearization to obtain a DNA fragment, and a cRNA is synthesized employing the DNA fragment as a template and using mMESSAGE mMACHINE T7 Ultra (manufactured by Ambion) according to the attached instructions. The prepared cRNA is injected at 10 ng per oocyte using a nanoliter injector manufactured by Drummond Scientific (Broomall, Pa., USA). This is transiently expressed after injection of a cRNA.

The insect voltage-gated potassium channel comprising an amino acid sequence shown in the group B can be used as a research tool. For example, the channel can be used as a research tool for implementing study such as the assaying the pesticidal activity and screening of a chemical substance having the pesticidal activity. In addition, for example, also in study of analyzing an action mechanism of an agent acting on the voltage-gated potassium channel, the voltage-gated potassium channel can be utilized as a research tool.

In addition, polynucleotides encoding amino acid sequences shown in the group B and polynucleotides having a nucleotide sequence having complementarity to them, as well as partial nucleotide sequences of polynucleotides encoding amino acid sequences shown in the group B, or polynucleotides having nucleotide sequences having complementarity to the partial nucleotide sequences, and a polynucleotide complying a nucleotide sequence represented by SEQ ID NO: 4 or 5 can be used as a research tool. For example, a part of them functions as a polynucleotide used in a method of preparing a voltage-gated potassium channel as described above.

In addition, a polynucleotide encoding an amino acid sequence shown in the group B or a polynucleotide comprising a nucleotide sequence complementary to the same, or a partial nucleotide sequence of a polynucleotide encoding an amino acid sequence shown in the group B, or a polynucleotide comprising a nucleotide sequence complementary to the partial nucleotide sequence, and a polypeptide comprising the nucleotide sequence shown in any of SEQ ID NO:11 to 15 can be used as a research tool. For example, a part of them functions as a polynucleotide used in a process for producing the voltage-gated potassium channel as described above.

Alternatively, the part can be used as an important research tool for obtaining a polynucleotide shown in the polynucleotide group B using PCR, or obtaining a polynucleotide shown in the polynucleotide group B using hybridization, as described above.

In the present invention, the “method of measuring an ability of a test substance to modulate the activity of an insect voltage-gated potassium channel comprising: a first step of measuring the electrophysiological activity of a voltage-gated potassium channel of a cell that expresses a voltage-gated potassium channel selected from among the group A in a form functional as an ion channel in a system in which the cell contact with a test substance; and a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the cell in a system containing no test substance” indicates a method characterized by comprising the first step and the second step, in a variety of methods of testing the ability to modulate an activity of the voltage-gated potassium channel of a test substance.

Herein, the group A is a protein comprising the aforementioned amino acid sequence.

The first step is a “step of adding a test substance to a reaction system for measuring the electrophysiological activity of a cell expressing the voltage-gated potassium channels in a form functional as an ion channel to bring the cell into contact with the test substance, and measuring the electrophysiological activity of the cell”.

In addition, the second step is a step of comparing the activity at measurement of the test substance and the activity of a control, and assessing the ability to modulate the activity of the voltage-gated potassium channel based on the difference.

Herein, the control means, for example, when a test substance dissolved in a solvent is added to the reaction system, a test section in which only a solvent same as that used to dissolve the test substance is added.

The cell expressing the voltage-gated potassium channel in a form functional as an ion channel used in the method of measuring the ability to modulate an activity of the voltage-gated potassium channel of a test substance comprising the first step and the second step is a cell expressing a protein shown in the group A in a form functional as an ion channel.

Examples of the cell expressing a protein shown in the group A in a form functional as an ion channel include specifically an oocyte from Xenopus laevis expressing a protein shown in the group A in a form functional as an ion channel.

In the present invention, the “method of measuring an ability of a test substance to modulate the activity of an insect voltage-gated potassium channel comprising: a first step of measuring the electrophysiological activity of a voltage-gated potassium channel of a nematode that expresses a voltage-gated potassium channel selected from among the group A in a form functional as an ion channel in a system in which the nematode contact with a test substance; and a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the nematode in a system containing no test substance” indicates a method characterized by comprising the first step and the second step, in a variety of methods of testing the ability to modulate an activity of the voltage-gated potassium channel of a test substance.

Herein, the group A is a protein comprising the aforementioned amino acid sequence.

The first step is a “step of adding a test substance to a reaction system for measuring the electrophysiological activity of a nematode expressing the voltage-gated potassium channels in a form functional as an ion channel to bring the nematode into contact with the test substance, and measuring the electrophysiological activity of the nematode”.

In addition, the second step is a step of comparing the activity at measurement of the test substance and the activity of a control, and assessing the ability to modulate the activity of the voltage-gated potassium channel based on the difference.

Herein, the control means, for example, when a test substance dissolved in a solvent is added to the reaction system, a test section in which only a solvent same as that used to dissolve the test substance is added.

The nematode expressing the voltage-gated potassium channel in a form functional as an ion channel used in the method of measuring the ability to modulate an activity of the voltage-gated potassium channel of a test substance comprising the first step and the second step is a nematode expressing a protein shown in the group A in a form functional as an ion channel.

Examples of the nematode expressing a protein shown in the group A in a form functional as an ion channel include specifically Caenorhabdtis elegans expressing a protein shown in the group A in a form functional as an ion channel.

Particularly, upon implementation of screening of a pestcidal agent, they can be used as an experimental tool for an experiment which is performed for screening. Specifically, they can be used as an experimental tool for an experiment which is performed upon implementation of the assaying of a pestcidal ability, screening of a chemical substance having a pestcidal ability, and the like.

Further, the present invention also includes a system which comprises a means to input, store and manage data information of an ability of test substances, wherein said ability is an ability to modulate the activity of an insect voltage-gated potassium channel (hereinafter, referred to as means a in some cases), a means to query and retrieve the data information based on a desired criterion (hereinafter, referred to as means b in some cases), and a means to display and output the result which is queried and retrieved (hereinafter, referred to as means c in some cases) (hereinafter, referred to as present system in some cases).

First, a means a will be explained. A means a is a means to, after data information of an ability to modulate the activity of an insect voltage-gated potassium channel possessed by the test substance is inputted, store and manage the inputted information, as described above. The information is inputted by an inputting means 1, and is usually memorized in a memory means 2. Examples of an inputting means include means which can input the information such as a keyboard and a mouse. When inputting and storing ·□managing of the information are completed, a procedure progresses to a next means b. For storing ·□managing the information, a large amount of data may be effectively stored and managed by inputting information having a data structure using a hardware such as a computer, and a software such as OS and database management, and storing the information into a suitable memory device, for example, computer-readable recording medium such as a flexible disc, a photomagnetic disc, CD-ROM, DVD-ROM, and a hard disc.

A means b will be explained. A means b is a means to query and retrieve the data information stored and managed by a means of a based on criterion for obtaining a desired result, as described above. For the information, when criterion for querying and retrieving is inputted by an inputting means 1, and information in conformity with the criterion is selected among the information usually memorized in a memory means 2, a procedure progresses to a next means c. The selected result is usually memorized in a memory means 2 and, further, can be displayed by a displaying·outputting means 3.

A means c will be explained. A means c is a means to display and output the result which is queried and retrieved, as described above. Examples of the displaying ·□outputting means 3 include a display, a printer and the like, and the result may be displayed on a display device of a computer, or may be outputted on a paper by printing.

### EXAMPLES

The present invention will be explained in more detail below by way of Examples, but the present invention is not limited to these particular Examples.

### Example 1

### Extraction of Total RNA from Cotton Aphid and German Cockroach

(1) Extraction of Total RNA from Cotton Aphid.

A population of adults and larvae of cotton aphid (Aphis gossypii), which had been reared on leaves of potted cucumber, was scraped from the surface of the leaves with a small brush, and 630 mg of the obtained population was crushed into a powder in liquid nitrogen using a mortar and a pestle. From the resulting frozen crushed powder, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 9° C. for 5 minutes. After the supernatant was removed, the precipitate of total RNA was slightly dried and then dissolved in 1 ml of commercially available RNase-free water (Nacalai Tesque, Inc.). A concentration of the prepared total RNA (calculated from an absorbance at 260 nm) was 6.9 mg/ml.

(2) Extraction of Total RNA from German Cockroach

Adults, nymphs and oothecae of artificially-reared German cockroach (Blattella germanica) were provided as samples. Ten (10) of adult males and 10 of adult females (individuals from each of which ootheca has been removed) were used as an adult sample of 1.1 g, 10 of nymph males and 10 of nymph females were used as a nymph sample of 1.0 g, and 26 oothecae were used as an ootheca sample of 1.0 g. Three kinds of these samples were separately crushed into a powder in liquid nitrogen using separate mortars and pestles. From each of the resulting frozen crushed powders, RNA was isolated using a RNA extracting reagent ISOGEN (manufactured by Nippon Gene) as follows. After 10 ml of ISOGEN was added to the frozen crushed powder in the mortar, the crushed powder was ground for 10 minutes while kept on ice. After grinding, a fluid sample was transferred to a 15 ml tube with a pipette, and 2 ml of chloroform (manufactured by Wako Pure Chemical Industries, Ltd.) was added thereto. Immediately, the mixture was vigorously shaken for 15 seconds and then left at rest at room temperature for 3 minutes. Then, the resulting mixture was centrifuged at 12,000×g at 4° C. for 15 minutes, and each 5 ml of aqueous layer were transferred to two new tubes. After 5 ml of ISOGEN was added to each tube, the mixture was immediately shaken vigorously for 15 seconds, and left at rest at room temperature for 3 minutes. Then, the resulting mixture was centrifuged at 12,000×g at 4° C. for 15 minutes, and each 10 ml of aqueous layer were transferred to new 50 ml tubes, respectively. Subsequently, 10 ml of isopropanol (manufactured by Wako Pure Chemical Industries, Ltd.) was added to each tube, and the mixture was kept on ice for 30 minutes. The resulting mixture was centrifuged at 12,000×g at 4° C. for 10 minutes to precipitate RNA. After the supernatant was removed, 20 ml of 70% ethanol was added to the residue. The resulting mixture was centrifuged at 10,000×g at 4° C. for 5 minutes. After the supernatant was removed, the precipitate of total RNA was slightly dried and then dissolved in 1 ml of commercially available RNase-free water (Nacalai Tesque, Inc.). A concentration of the prepared total RNA (calculated from absorbance at 260 nm) was 1.1 mg/ml in the case of adult-derived total RNA, was 2.5 mg/ml in the case of nymph-derived total RNA, and 1.4 mg/ml in the case of ootheca-derived total RNA.

### Example 2

### Isolation of Cotton Aphid Seizure Gene

First-strand cDNA was prepared using total RNA from cotton aphid, random Primers (Invitrogen) and Superscript III (Invitrogen) for RT-PCR according to the manufacturer's procedure of Superscript III.

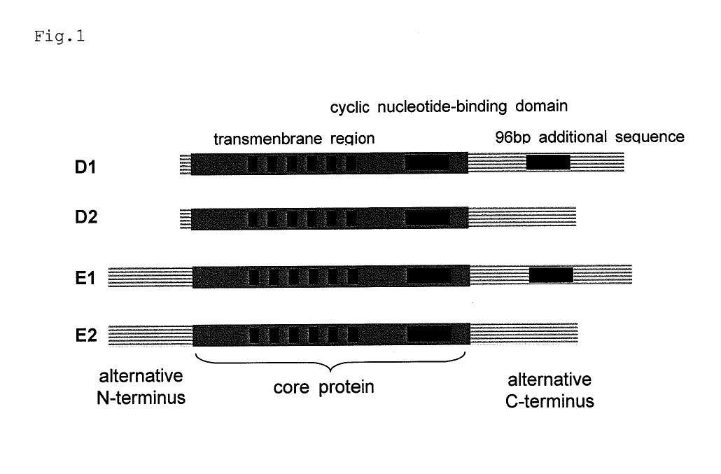

A full-length cDNA of cotton aphid seizure was amplified by PCR using an oligonucleotide comprising the nucleotide sequence of SEQ ID NO: 11 and an oligonucleotide comprising the nucleotide sequence of SEQ ID NO: 12, which are primers specific for the gene, and Expand Long Template PCR System (manufactured by Roche) according to the manufacturer's procedure. The cDNA described in Example 1 was used as template. The PCR conditions used were those for touchdown PCR as follows: an initial denaturation at 94° C. for 2 minutes; followed by 10 cycles of touchdown-PCR, one cycle being 94° C. for 30 seconds, 70° C. for 30 seconds with a decrease of 1° C. per cycle, and 68° C. for 4 minutes; followed by 25 cycles of PCR, one cycle being 94° C. for 30 seconds, 60° C. for 30 seconds, and 68° C. for 30 seconds extended with 10 seconds each cycle; and followed by 68° C. for 7 minutes. The resulting PCR products were analyzed and purified by agarose gel electrophoresis to obtain an objective DNA fragment. Further, the thus obtained DNA fragment was cloned into a TOPO XL vector (manufactured by Invitrogen), a nucleotide sequence was determined, and there were four kinds of splice variants of D1, D2, E1 and E2 (D1: SEQ ID NO: 2, D2: SEQ ID NO: 4, E1: SEQ ID NO: 6, E2: SEQ ID NO: 8). The tour kinds of nucleotide sequences are schematically shown in FIG. 1. The D and E variants differ in their 5′ region and the 5′ region of E variants were longer by about 270 bp than that of D isoforms. The D1 and E1 variants have an additional sequence of 96 base pairs compared to the D2 and E2 variants, which is located 1 kb from the 3′ region. This additional sequence is herein represented as SEQ ID NO 9. Amino acid sequences deduced from the nucleotide sequences of SEQ ID NO: 2, 4, 6 and 8 were amino acid sequences of SEQ ID NO: 1, 3, 5 and 7 respectively. Untranslated regions at the 5′ end and the 3′ end are common, and the nucleotide sequence of D1 containing the untranslated regions is shown in SEQ ID NO:10.

### Example 3

### Isolation of German Cockroach Seizure Gene

For isolation of homologues of seizure gene from other insect species such as German cockroach (Blatella germanica), degenerate primers are designed using Codehop program (publicly accessible on the website of Blocks Protein Analysis Server operated within the Fred Hutchinson Cancer Research Center at http://blocks.fhcrc.org/blocks/codehop.html), referring to the amino acid sequence of the aforementioned cotton aphid seizure and the previously-known amino acid sequences of homologues from human (NCBI accession number Q12809 and Q9H252), Caenorhabditis elegans (NP_{—}49782), Drosophila melanogaster (AAM68296), Anopheles gambiae (XP_{—}308166).

Partial sequences of a homologue of seizure gene of a selected insect species are amplified by a series of PCR using first-strand cDNA derived from the insect species as a template. Herein, the first-strand cDNA as a template is prepared by the aforementioned method using Superscript III. Amplification by PCR is performed using a set of degenerate primers as a forward primer and a reverse primer as well as Amplitaq Gold (manufactured by Applied Biosystems) according to the manufacturer's procedure annexed to the reagent. The PCR conditions are those for touchdown PCR as follows: an initial denaturation at 94° C. for 5 minutes; followed by 10 cycles of touchdown-PCR, one cycle being 94° C. for 30 seconds, 60° C. for 1 minute with a decrease of 1° C. per cycle, and 72° C. for 1 minute and 30 seconds; followed by 25 cycles of PCR, one cycle being 94° C. for 30 seconds, 50° C. for 30 seconds, and 72° C. for 1 minute and 30 seconds; and followed by 72° C. for 7 minutes. The PCR product is analyzed and purified by agarose gel electrophoresis to obtain DNA of interest. Further, the obtained DNA is cloned into the pCR4-TOPO vector (manufactured by Invitrogen), and sequenced.

Thus, partial sequence of a seizure gene of Blatella germanica is obtained.

Then, primers specific for the resulting partial sequences of the insect homologue of seizure gene are designed, and 3′RACE PCR or 5′RACE PCR is performed in order to obtain a full-length sequence of the gene. The 3′RACE PCR is performed employing first-strand cDNA prepared from the insect total RNA as a template and using SMART PCR cDNA Synthesis Kit (manufactured by Clontech) according to the manufacturer's instructions annexed to the kit. The 5′ RACE PCR is performed employing first-strand cDNA prepared from the insect total RNA as a template and using 5′/3′ RACE Kit, 2″ Generation (manufactured by Roche) according to the manufacturer's instructions annexed to the kit.

In 5′RACE reaction, a reverse primer specific for the sequence of interest is used in combination with Oligo-d(T)-anchor primer1 contained in 5′/3′ RACE Kit, 2nd Generation as a forward primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 40 seconds; followed by 25 cycles of PCR, one cycle being 95° C. for 30 seconds, 50° C. for 30 seconds, and 72° C. for 40 seconds extended with 20 seconds each cycle; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

When a distinct amplification product is not obtained by the first-round PCR, nested PCR is performed using the first-round PCR product as a template. As primers, a specific reverse primer which is designed to bind to internal sequence of the first-round PCR product is used in combination with PCR Anchor primer contained in 5′/3′ RACE Kit, 2nd Generation as a forward primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 40 seconds; followed by 25 cycles of PCR, one cycle being 95° C. for 30 seconds, 50° C. for 30 seconds, and 72° C. for 40 seconds extended with 20 seconds each cycle; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

In 3′ RACE reaction, a forward primer specific for the sequence of interest is used in combination with universal primer mix (UPM) contained in SMART PCR cDNA Synthesis Kit as a reverse primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 1 minute; followed by 25 cycles of PCR, one cycle being 95° C. for 30 seconds, 50° C. for 30 seconds, and 72° C. for 1 minute; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

When a distinct amplification product is not obtained by the first-round PCR, nested PCR is performed using the first-round PCR product as a template. As primers, a specific forward primer which is designed to bind to internal sequence of the first-round PCR product is used in combination with NUP primer contained in SMART PCR cDNA Synthesis Kit as a reverse primer. The PCR conditions are those for touchdown PCR as follows: an initial denaturation at 95° C. for 5 minutes; followed by 10 cycles of touchdown-PCR, one cycle being 95° C. for 30 seconds, 60° C. for 30 seconds with a decrease of 1° C. per cycle, and 72° C. for 1 minute; followed by 25 cycles of PCR, one cycle being 95° C. for 30 seconds, 50° C. for 30 seconds, and 72° C. for 1 minute; and followed by 72° C. for 7 minutes. The resulting PCR product is analyzed and purified by agarose gel electrophoresis to obtain DNA of interest. Further, the obtained DNA is cloned into the pCR4-TOPO vector (manufactured by Invitrogen), and sequenced.

The above sequencing results reveal 5′-terminal sequence and 3′-terminal sequence, each encoding N-terminal region and C-terminal region of the insect seizure, respectively.

### Example 4

### Construction of Plasmid for Producing Transgenic Nematode

A seizure gene fragment from Aphis gossypii to be cloned into a vector for producing a recombinant nematode was amplified by PCR employing splice variants D1, D2, E1 and E2 of cotton aphid seizure cloned into a TOPO XL vector (manufactured by Invitrogen) shown in Example 2, respectively, as a template, and using primers specific for respective gene sequences.

In the case of D1, the fragment was amplified by PCR using an oligonucleotide consisting of a nucleotide sequence of SEQ ID NO: 13 and an oligonucleotide consisting of a nucleotide sequence of SEQ ID NO: 14 which are primers specific for a gene sequence, by PerfectShot ExTaq (manufactured by Takara Bio Inc.) according to instructions attached to the reagent. The condition of PCR was touchdown PCR described later. That is, first, 94° C. for 5 minutes, subsequently, 94° C. for 30 seconds, from 80° C. for 30 seconds accompanying 1° C. decrease per cycle, and 72° C. for 4 minutes were 10 cycles and, thereafter, further, 94° C. for 30 seconds, 70° C. for 30 seconds, and 72° C. for 4 minutes were 25 cycles and, finally, 72° C. for 7 minutes. The PCR product was analyzed by agarose gel electrophoresis, and an objective DNA fragment was cloned into a TOPO XL vector (manufactured by Invitrogen) to obtain TOPO-XL-D1.

Since the primer of SEQ ID NO: 13 contains a NheI restriction enzyme site, and the primer of SEQ ID NO: 14 contains a NcoI restriction enzyme site, a vector pDW2700 (manufactured by Devgen, described in WO 2003097682) for producing a recombinant nematode containing TOPO-XL-D1 and a myo-2 promoter derived from a nematode was cut with NheI and NcoI. The NheI/NcoI DNA fragment of an ERG-type voltage-gated potassium channel D1 of cotton aphid, and a vector pDW2700 for producing a recombinant nematode digested with NheI/NcoI were analyzed by agarose gel electrophoresis, and purified by Quiaquick Gel Extraction Kit (manufactured by Qiagen). Using Takara ligase I (manufactured by Takara Bio Inc.), both purified DNA fragments were ligated to obtain pGBB010.

In the case of D2, the fragment was amplified by PCR with PerfectShot ExTaq (manufactured by Takara Bio Inc.) using an oligonucleotide consisting of a nucleotide sequence of SEQ ID NO: 13 and an oligonucleotide consisting of a nucleotide sequence of SEQ ID NO: 14 which are primers specific for a gene sequence according to instructions attached to the reagent. The condition of the PCR was touchdown PCR described later. That is, first, 94° C. for 5 minutes, subsequently, 94° C. for 30 seconds, from 80° C. for 30 seconds accompanying 1° C. decrease per cycle, and 72° C. for 4 minutes were 10 cycles and, thereafter, further, 94° C. for 30 seconds, 70° C. for 30 seconds, and 72° C. for 4 minutes were 25 cycles and, finally, 72° C. for 7 minutes. The PCR product was analyzed by agarose gel electrophoresis, and an objective DNA fragment was cloned into a TOPO XL vector (manufactured by Invitrogen) to obtain TOPO-XL-D2.

Since the primer of SEQ ID NO: 13 contains a NheI restriction enzyme site, and the primer of SEQ ID NO: 14 contains a NcoI restriction enzyme site, a vector pDW2700 (manufactured by Devgen) for producing a recombinant nematode containing TOPO-XL-D2 and a myo-2 promoter was cut with NheI and NcoI. A NheI/NcoI DNA fragment of cotton aphid seizure D2, and a vector pDW2700 for producing a recombinant nematode digested with NheI/NcoI were analyzed by agarose gel electrophoresis, and purified by Quiaquick Gel Extraction Kit (manufactured by Qiagen). Using Takara ligase I (manufactured by Takara Bio Inc.), both purified DNA fragments were ligated to obtain pGBB014.

In the case of E1 and E2, the fragment was amplified by PCR with Takara LA Taq (manufactured by Takara Bio Inc.) using an oligonucleotide consisting of a nucleotide sequence of SEQ ID NO: 15 and an oligonucleotide consisting of a nucleotide sequence of SEQ ID NO: 14 which are primers specific for a gene sequence according to instructions attached to the reagent. The condition of the PCR was touchdown PCR described later. That is, first, 94° C. for 5 minutes, subsequently, 94° C. for 30 seconds, from 70° C. for 30 seconds accompanying 1° C. decrease per cycle, and 72° C. for 4 minutes were 10 cycles and, thereafter, further, 94° C. for 30 seconds, 60° C. for 30 seconds, and 72° C. for 30 seconds accompanying 10 seconds increase per cycle were 25 cycles and, finally, 72° C. for 7 minutes. The PCR product was analyzed by agarose gel electrophoresis, and an objective DNA fragment was cloned into a TOPO XL vector (manufactured by Invitrogen) to obtain TOPO-XL-E1-MUT and TOPO-XL-E2-MUT. However, as a result of sequence analysis, a clone entirely consistent with nucleotide sequences of E1 and E2 of a template was not obtained. Then, utilizing a combination of D1 and E1, and D2 and E2 having the same nucleotide sequence of regions except for a 5′ end, TOPO-XL-E1-MUT and TOPO-XL-E2-MUT were altered using the TOPO-LX-D1 and TOPO-LX-D2 by the following operation.

Since TOPO-XL-D1 and TOPO-XL-E1-MUT contain a HindIII restriction enzyme site on a TOPO XL vector (manufactured by Invitrogen), and contain a BsgI restriction enzyme site on a 5′ upstream region of D1 and E1 genes, a 5′ upstream region fragment flanked by HindIII and BsgI restriction enzyme sites was exchanged. First, TOPO-XL-D1 was digested with HindIII and BsgI, analysis by agarose gel electrophoresis, and purification by Quiaquick Gel Extraction Kit (manufactured by Qiagen) were performed, and the HindIII/BsgI fragment containing a 5′ upstream region of D1 was removed. Subsequently, TOPO-XL-E1-MUT was digested with HindIII and BsgI, analysis by agarose gel electrophoresis, and purification by Quiaquick Gel Extraction Kit (manufactured by Qiagen) were performed, and the HindIII/BsgI fragment containing a 5′ upstream region of E1 was obtained. Since a region downstream of the BsgI restriction enzyme site was common to D1 and E1, the resulting HindIII/BsgI fragment containing a 5′ upstream region of E1, and TOPO-XL-D1 from which a 5′ upstream region of D1 had been removed were ligated, to obtain TOPO-XL-E1 having a correct nucleotide sequence of E1.

Since TOPO-XL-D2 and TOPO-XL-E2-MUT contain a HindIII restriction enzyme site on a TOPO XL vector (manufactured by Invitrogen), and contain a BsgI restriction enzyme site on a 5′ upstream region of D2 and E2 genes, a 5′ upstream region fragment flanked by HindIII II and BsgI restriction enzyme sites was exchanged. First, TOPO-XL-D2 was digested with HindIII and BsgI, analysis by agarose gel electrophoresis, and purification by Quiaquick Gel Extraction Kit (manufactured by Qiagen) were performed, and a HindIII/BsgI fragment containing a 5′ upstream region D2 was removed. Subsequently, TOPO-XL-E2-MUT was digested with HindIII and BsgI, analysis by agarose gel electrophoresis, and purification by Quiaquick Gel Extraction Kit (manufactured by Qiagen) were performed, and a HindIII/BsgI fragment containing a 5′ upstream region of E2 was obtained. Since regions downstream of a BsgI restriction enzyme site are common to D2 and E2, the resulting HindIII/BsgI fragment containing a 5′ upstream region of E2, and TOPO-XL-D2 from which a 5′ upstream region of D2 had been removed were ligated using Takara ligase I (manufactured by Takara Bio Inc.) to obtain TOPO-XL-E2 having a correct nucleotide sequence of E2.

Since E1 and E2 genes contained in TOPO-XL-E1 and TOPO-XL-E2 are flanked by a NheI restriction enzyme site and a NcoI restriction enzyme site, TOPO-XL-E1 and TOPO-XL-E2 were cut with NheI and NcoI. A vector pDW2700 (manufactured by Devgen) for producing a recombinant nematode was similarly cut with NheI and NcoI. The NheI/NcoI DNA fragment of E1 and E2, and pDW2700 digested with NheI/NcoI was analyzed by agarose gel electrophoresis, and purified by Quiaquick Gel Extraction Kit (manufactured by Qiagen). The NheI/NcoI DNA fragment of E1 and pDW2700, or the NheI/NcoI DNA fragment of E2 and pDW2700 were ligated, respectively, using Takara ligase I (manufactured by Takara Bio Inc.).

### Example 5

### Construction of Plasmid for Electrophysiological Experiment

TOPO-XL-D1, TOPO-XL-D2, TOPO-XL-E1, and TOPO-XL-E2 shown in Example 4 were treated with a restriction enzyme NheI, and a cohesive end was blunted with a Klenow enzyme. After further digestion with a restriction enzyme EcoRI, analysis by agarose gel electrophoresis, and purification by Quiaquick Gel Extraction Kit (manufactured by Qiagen) were performed, each DNA fragment of four kinds of cotton aphid seizure splice variants (D1, D2, E1, E2) was obtained. A vector pcDNA3.1(+)-X for an electrophysiological experiment (manufactured by IonGate, a vector in which 5′ and 3′ non-translated regions of a-blobin gene derived from Xenopus laevis were inserted into a vector pcDNA3.1(+) manufactured by Invitrogen) was treated with a restriction enzyme BamHI, and a cohesive end was blunted with a Klenow enzyme. This was further digested with a restriction enzyme EcoRI, and analysis by agarose gel electrophoresis, and purification by Quiaquick Gel Extraction Kit (manufactured by Qiagen) were performed. Each DNA fragment of the D1, D2, E1 and E2, and pcDNA3.1(+)-X were ligated, respectively, using Takara ligase I (manufactured by Takara Bio Inc.) to obtain pcDNA3.1X-D1, pcDNA3.1X-D2, pcDNA3.1X-E1, and pcDNA3.1X-E2. These four kinds of plasmids were used in an electrophysiological experiment by the two-electrode voltage clamp method (TEVC method) using an oocyte of Xenopus laevis.

The pGBB014 containing seizure D2 of cotton aphid shown in Example 4 was treated with a restriction enzyme NheI, and a cohesive end was blunted with Mung Bean Nuclease (manufactured by New England Biolabs). After further digestion with a restriction enzyme XhoI, analysis by agarose gel electrophoresis, and purification by Quiaquick Gel Extraction Kit (manufactured by Qiagen) were performed. On the other hand, a vector pcDNA3(+) manufactured by Invitrogen was treated with restriction enzymes EcoRV and XhoI, and analysis by agarose gel electrophoresis, and purification by Quiaquick Gel Extraction Kit (manufactured by Qiagen) were performed. The DNA fragment of D2, and pcDNA3(+) were ligated using Takara ligase I (manufactured by Takara Bio Inc.) to obtain pGBB036. This plasmid was used in an electrophysiological experiment by a patch clamp method using a CHO cell.

### Example 6

### Electrophysiological Analysis by TEVC Method Using Xenopus laevis Oocyte)

Regarding four kinds of cotton aphid seizure splice variants (D1, D2, E1, E2), a gene optimal for producing a gene-recombinant nematode was selected by electrophysiological analysis by the two-electrode voltage clamp method (TEVC method) using an oocyte of Xenopus laevis.

In order to continuously maintain circulation and cleaning of water, female Xenopus laevis was reared in a water bath equipped with an air pump and a filtration device. For Xenopus laevis, a laboratory rearing line was purchased from NASCO (Fort Atkinson, Wis., USA), and adapted to a dry sample, and used in an experiment. Xenopus laevis was reared in tap water retained at a water temperature of 18° C., and a light/dark cycle of each 12 hours was artificially given. Xenopus laevis was anesthetized using a 2 g/l tolycaine aqueous solution, and subsequently cooled with an ice. A lower abdominal part was excised small, and an ovary containing an oocyte was removed. A part of ovarian follicle was excised, washed with an OR2 solution (82.5 mM NaCl, 2 mM KCl, 1 mM MgCl₂, 5 mM HEPES, pH 7.4) containing no calcium ion, and subjected to 2 mg/ml collagenase treatment at 16° C. to remove a follicle cell from a surface of an oocyte. After collagenase treatment, an oocyte was washed with an OR2 solution, and stored in an OR2 solution with a final concentration 50 mg/l Gentamycin added thereto.

Four kinds of plasmids (pcDNA3.1X-D1, pcDNA3.1X-D2, pcDNA3.1X-E1, pcDNA3.1X-E2) for an electrophysiological experiment produced in Example 5 were digested with a restriction enzyme XbaI for linearization to obtain a DNA fragment, and a cRNA was synthesized employing the DNA fragment as a template, and using mMESSAGE mMACHINE T7 Ultra (manufactured by Ambion) according to the attached instructions. The prepared cRNA was injected at 10 ng per oocyte using a nanoliter injector manufactured by Drummond Scientific (Broomall, Pa., USA). The oocyte after cRNA injection was cultured at 16° C. for 1 to 6 days in an OR2 solution with Gentamycin added thereto to express an objective gene.

All electrophysiological experiments were performed in a ND96 standard solution (96 mM NaCl, 2 mM KCl, 1 mM MgCl₂, 1.8 mM CaCl₂, 5 mM HEPES, pH 7.4). A test compound was dissolved in DMSO at a concentration of 100 mM, and diluted with a ND96 standard solution to an objective concentration immediately before use. A DMSO concentration in a test system at a maximum test concentration (100 μM) was 0.1%. This DMSO concentration did not influence on measurement of a current.

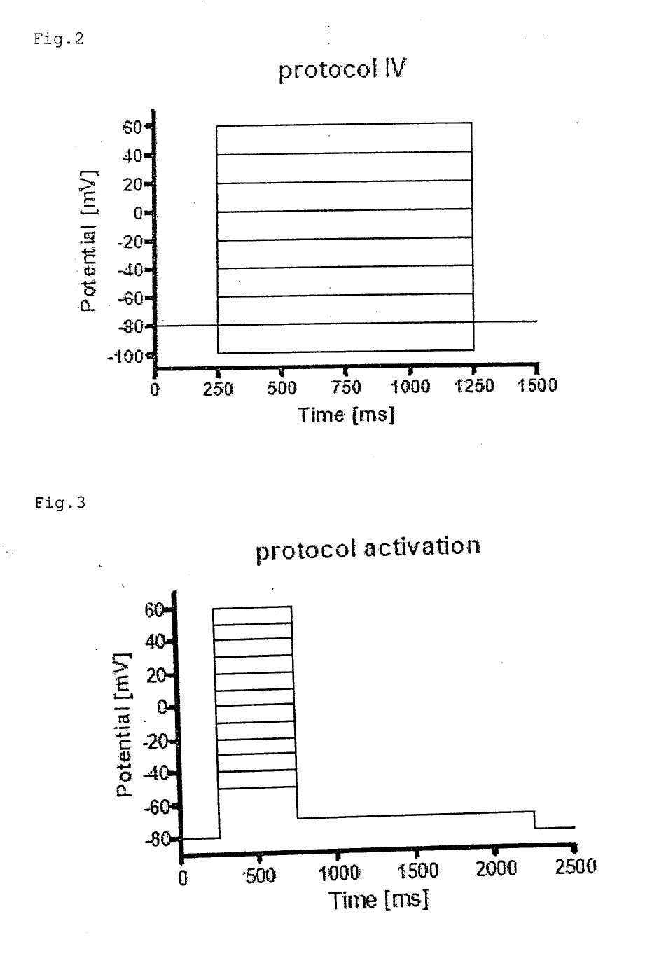

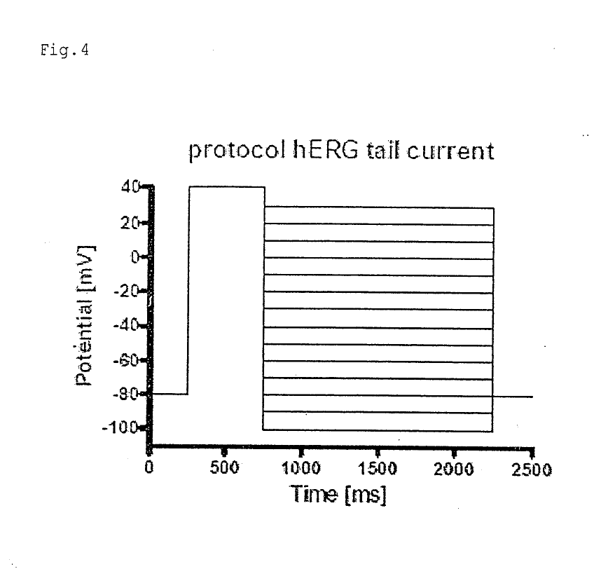

Using an amplifier TEC-03X manufactured by npi electronics (Tamm, Germany), and software Cellworks, an electrophysiological measurement was performed by the two-electrode voltage clamp method. Experimental data was recorded at room temperature (20 to 25° C.). A microelectrode was filled with 3 M KCl, and an electric resistance value was set to be 0.2 to 1.5 MΩ. Throughout recording, the oocyte was brought into the state where it is perfused with ND96. While a potential pulse was given, the oocyte was fixed at a retention potential of −80 mV. A test protocol is shown in FIGS. 2 to 4. As a standard protocol, protocol IV was used for recording a current voltage relationship of an ion channel. Protocol activation and protocol hERG tail current was used in order to confirm whether a channel exhibits the similar behavior to that of human ERG.

An activity of a compound was measured using protocol IV by the following procedure. First, a continuous pulse of protocol IV was repeatedly imparted to the oocyte perfused with ND96 three times. Then, in the presence of a compound dissolved in ND96, this continuous pulse was repeatedly imparted to the oocyte six times. When the experimental condition was sufficient, subsequently, an experiment of washing out a test compound was performed. In the washing out experiment, the oocyte was perfused with ND96 again, and this continuous pulse was repeatedly imparted three to six times.

At each test concentration, an amplitude of a current waveform in last three times of continuous pulses was averaged, and a calculated current value was used for calculating an inhibition degree. Using a current value at measurement with only ND96 as a standard, data was corrected. For data analysis, software

Excel manufactured by Microsoft (Redmond, Wash., USA), and software Origin manufactured by OriginLac (Northampton, Mass., USA) were used. An IC50 value was obtained using a Hill's equation.

Using protocol IV, whether four kinds of splice variants were expressed as a functional potassium channel was confirmed. As shown in Table 3, D1, D2 and E2 were expressed as a functional potassium channel, but E1 did not exhibit an activity in this test system. Since D2 exhibited the maximum current response, this was determined to be optimal for producing a recombinant nematode used in selection of a compound.

TABLE 3

| Splice-variant | Current size (10 ng cRNA) | |
|---|---|---|
| D1 | 0.8 ± 0.1 | μA |
| D2 | 23 ± 5 | μA |
| E1 | 0 | |
| E2 | 2.5 ± 0.6 | μA |

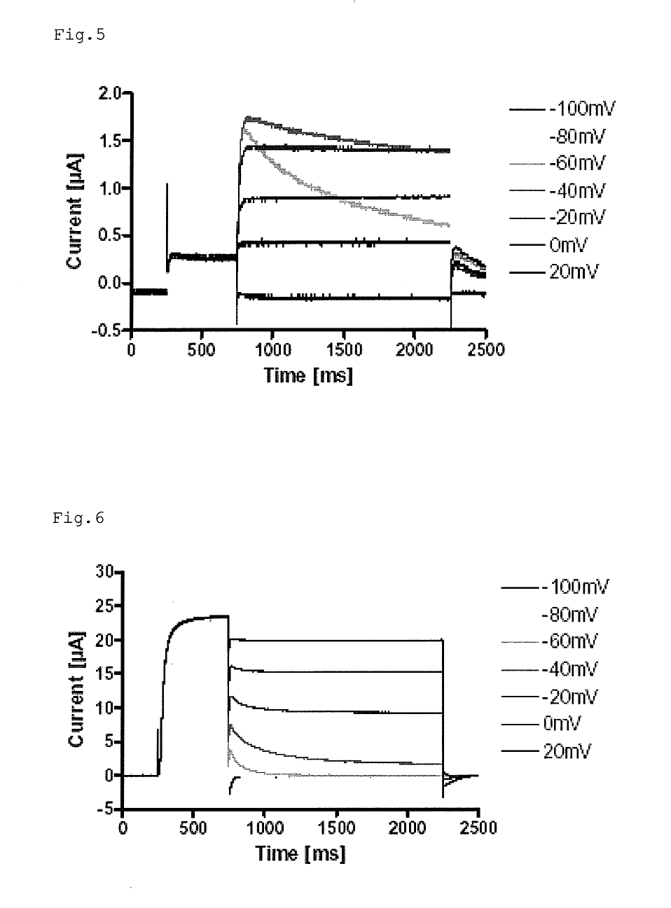

Then, when the presence or absence of inactivation was confirmed using protocol activation, D1, D2 and E2 exhibited little inactivation. Further, when the behavior was compared with that of human ERG using a protocol hERG tail current, a tail current peculiar in an ERG-type channel recognized in an electrophysiological response of human ERG of FIG. 5 was not recognized in an electrophysiological response of cotton aphid seizure D2 of FIG. 6. This result was common to D1 and E2. Although homology with drosophila seizure and human ERG is high, cotton aphid seizure exhibited a behavior which is electrophysiologically similar to that of an EAG channel.

Then, amino acid sequences associated with the function of a channel were compared. As shown in Table 4, Ser⁶²⁰ of human ERG was conserved in cotton aphid seizure, but to the contrary, Ala was situated in human EAG. However, Ser⁶³¹ of human ERG was substituted with Ala in cotton aphid seizure, and this Ala was also confirmed in human EAG. From the presence or absence of the tail current and comparison of amino acid sequences, cotton aphid seizure had the mixed characteristics of ERG and EAG.

TABLE 4

| hERG | hEAG | aphid seizure |
|---|---|---|
| Tail current | No tail current | No tail current |
| 620 Ser | Ala | Ser |
| 631 Ser | Ala | Ala |

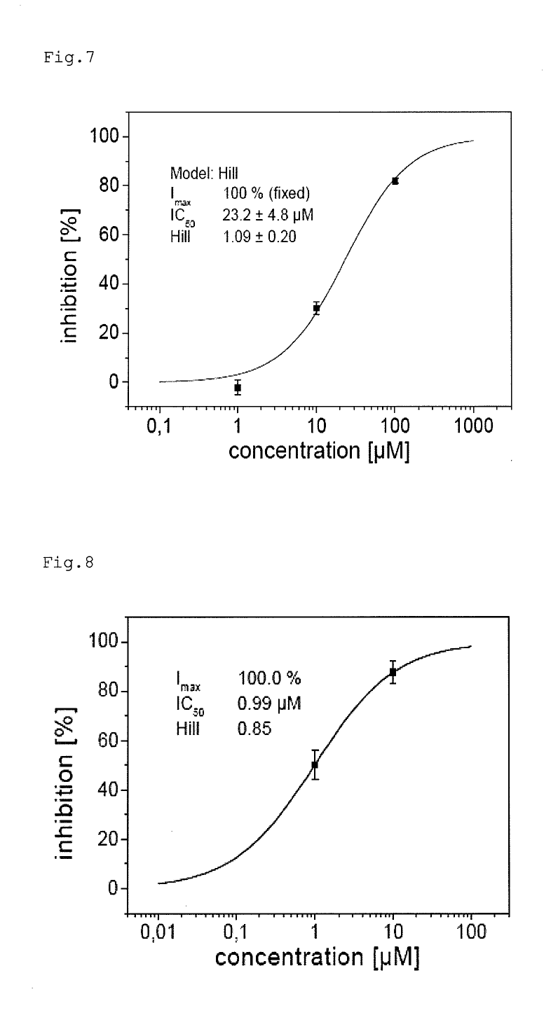

Finally, cotton aphid seizure D2 was pharmacologically analyzed. Dofetilide (FIG. 7) which is a selective blocker of hERG, and clofilium (FIG. 8) which acts on both of ERG/EAG as a blocker were used as a control agent. Dofetilide influenced on cotton aphid seizure D2 at an intermediate degree (IC50˜23 μM), while clofilium which acts on both of ERG/EAG as a blocker strongly acted on D2 (IC50˜1 μM). This was consistent with that D2 has the mixed characteristics of ERG/EAG. From the foregoing, it was shown that D2 functions as a voltage-gated potassium channel, and an ERG/EAG regulator influences on an activity of D2.

### Example 7

### Electrophysiological Analysis by Whole Cell Patch Clamp Method

Using a CHO cell expressing cotton aphid seizure D2, an activity of a voltage-gated potassium channel was measured by a whole cell patch clamp method.

According to a general tissue culturing procedure, pGBB036 produced in Example 5 was transiently introduced into the CHO cell. In order to confirm that gene introduction is successful, a GFP gene which is a fluorescent marker was simultaneously introduced into the CHO cell. A ratio of pGBB036 and the marker gene was 10:1. It was possible to simply identify the cell for which gene introduction was successful with an incident-light fluorescence microscope. After cultured for 24 hours, the gene-introduced cell was dispensed, frozen, and immediately cryopreserved at −150° C. On the day before implementation of an electrophysiological experiment, the cryopreserved cell was suspended in a fresh medium again, and the cell was seeded on a cover glass having a diameter of 12 mm which was spread in a petri dish. The petri dish was retained at a temperature of 37° C. under the condition of CO₂ at a 5% concentration. After 24 hours, the medium was exchanged, and the cover glass was transferred into a chamber of a patch clamp device.

Recording by the whole cell patch clamp method was implemented on one CHO cell. A microelectrode for a general patch clamp was prepared from a borosilicate glass capillary, and an electric resistance value was set at 5 to 7 MΩ. A solution in an electrode for whole cell recording was 130 mM of potassium gluconate, 2 mM of Na-G-gluconate, 20 mM of HEPES, 4 mM of MgCl₂, 4 mM of Na₂ATP, 0.4 mM of NaGTP, 5 mM of EDTA, pH 7.3. Recorded signals were all amplified with an amplifier PC-501A manufactured by Warner Instruments (Hamden, Conn., USA) and, thereafter, was digitalized at 10 kHz. For storing and analyzing data, WINWCP software (Version 3.5.2, Dr. J. Dempster, University of Strathclyde Electrophysiology Software), WINWDR software Version 2.7.0, Dr. J. Dempster, University of Strathclyde Electrophysiology Software), and Microsoft Office Excel 2003 were used.

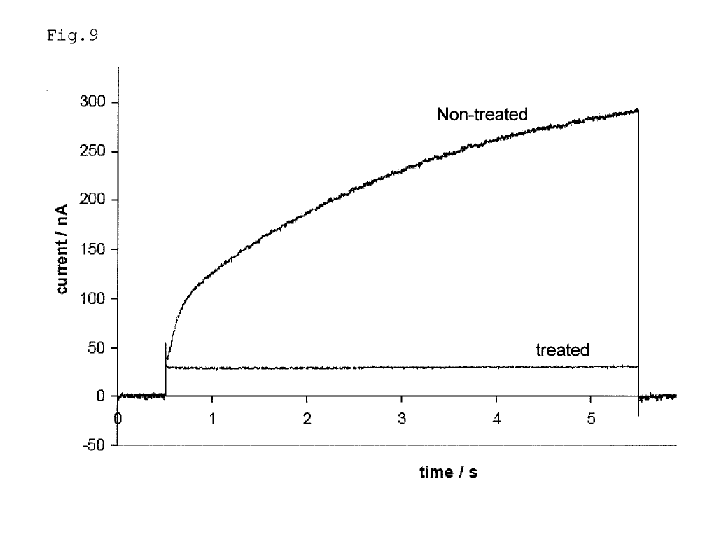

When the CHO cell expressing D2 was treated with 30 μM clofilium, as shown in FIG. 9, an activity of a channel was 86% inhibited in clofilium treatment relative to non-treatment.

### Example 8

### Production of Recombinant Nematode by Microinjection

A genetically engineered nematode in which cotton aphid seizure D2 has been introduced therein was produced by microinjection according to the following procedure to obtain UG2242 (genotype: pha-1 (e2123ts) III; bgEx1259 [pha-1; myo-2::ahid seizure (D2); myo-3::gfp]). UG2242 retained a gene in which cotton aphid seizure was connected downstream of a myo-2 promoter, as an extrachromosomal DNA array.

For microinjection, an agarose pad prepared by the following procedure was used as a specimen holder. Electrophoresis-grade agarose (manufactured by GIBCO BRL) was prepared to a concentration of 2%, and 20 μl thereof was added dropwise on a slide glass. Another slide glass was overlapped on the agarose added and, after ten seconds, the upper slide glass was removed. The slide glass on which agarose had been spread in a thin pad form was dried at 65° C. overnight. Using a needle puller PC-10 (manufactured by Narishige), a needle for microinjection was made from a borosilicate glass capillary GC120E-10 (manufactured by SDR). The needle was filled with 0.5 μl of a DNAmix. The DNA mix to be injected was prepared by mixing all of 5 ng of a vector pGBB014 containing a D2 gene to be introduced, 5 ng of a vector pBX (descried in Granato et al., Nucleic Acids Res. 22, 1762-1763, 1994) containing a pha-1 gene as a selection marker, 10 ng of a vector pDW2821 (in which a GFP gene is connected downstream of a myo-3 promoter derived from a nematode) containing a GFP gene as another marker gene, and 80 ng of a nematode genome DNA as a carrier DNA in 1 μl of a M9 buffer.

One droplet of a mineral oil (manufactured by Sigma, 400-5) was fallen on an agarose pad, and a nematode of a young ambisexual of a pha-1 mutant was placed into the oil. The needle for microinjection was attached to a micromanipulator (manufactured by Leitz). A focus was adjusted on the gonad and a tip of the needle while the oil was observed with an invert microscope (manufactured by Zeiss, Axiovert). The needle was stuck into the gonad, and the DNA mix was injected using Transjector 5246 (manufactured by Eppendorf). After completion of microinjection, 50 μl of a M9 buffer was added dropwise to recover the nematode. Finally, the nematode was transferred to a 3 cm NGM (Nematode Growth Medium) agar medium coated with Escherichia coli OP50 strain which is a feed of the nematode.

In order to use a pha-1 selection system, the nematode after microinjection (P0 generation) was reared at 15° C. to obtain a progeny (F1 generation). Then, F1 generation was collected, and transferred to 20 to 25° C., and a survived next generation was acquired as a transformant. This survived next generation was transferred from 15° C. to 20° C., a survival rate was obtained again, and the presence of a pha-1 selection system was confirmed. In addition, by visualization of a body wall muscle with GPF fluorescence, presence of a myo-3:: gfp marker gene was confirmed. By PCR, presence of the introduced cotton aphid seizure gene was confirmed.

By the similar method, it is possible to make a transformant of D1, E1 or E2.

UG2234 (genotype: pha-1 (e2123ts) III; bgEx1253 [pha-1; myo-3:: gfp]) which is a control line was also made by the similar method. Since this line has a pha-1 selection marker gene and a myo-3:: gfp marker gene, it was used in order to confirm these marker systems do not influence on an electropharyngeogram of a nematode shown in Example 10.

### Example 9

### Production of Recombinant Nematode by Particle Gun

According to the following procedure, a gene-recombinant nematode suitable for high throughput screening was made using a particle gun, and this was named as UG2346 (genotype: +/+; bgls1289 [myo-2::ahid seizure (D2); unc-119-unc-119]). UG2346 is a line in which a small copy number of a myo-2:: aphid seizure (D2) gene is incorporated into a genome.

UG2346 was made by a particle gun method. In order to make a line in which an introduction gene is incorporated into a genome using this technique, a gold particle obtained by coating an unc-119 mutant DP38 (genotype: unc-119 (ed3) III) with a DNA was fired. As the DNA for coating, a plasmid pDPMM#016b (described in Praitis et al., Genetics 157, 1217-1226, 2001) containing pGBB014 (myo-2:: aphid seizure (D2)) containing an cotton aphid seizure D2 gene to be introduced, and an UNC-119 fragment which is a selection system was used.

The UNC-119 mutant was cultured in a liquid medium. At the timepoint at which most of nematodes in the culturing solution were a last age larva (L4 larva) or a young imago, a nematode was recovered, and washed with 500 ml of a M9 buffer at least five times. Between respective washing procedures, the nematode was allowed to stand for 10 to 15 minutes to sediment. Thereafter, the nematode was transferred to a 50 ml tube, and sedimented again. The sedimented nematode (0.5 ml) was used in transformation. Into a 1.5 ml silicon-treated tube (manufactured by Scientific Plastics) were placed 15 mg of 1.5 to 3.0 micron gold particles (manufactured by Sigma-Aldrich). Added thereto was 1 ml of 70% ethanol, and this was stirred for at least 5 minutes using a vortex mixer. The mixture was allowed to stand for 15 minutes to sediment gold particles. After the mixture was rapidly centrifuged with a centrifuge, the supernatant was removed. Subsequently, 1 ml of deionized water was added, the mixture was stirred for 1 minute using a vortex mixer and allowed to stand for 1 minute to sediment gold particles, and the supernatant was removed. This operation was repeated three times. Finally, 250 μl of 50% glycerol was added, followed by stirring for 1 minute using a vortex mixer.

Each 2.5 μg of two kinds of plasmids of pDPMM#016b and pGBB014 was prepared and, after being linearized, this was purified with Qivaquick Spin Column (manufactured by Qiagen). A 1/10 amount of 7.5 M sodium acetate and a 2-fold amount of 100% ethanol were added, and the DNA was centrifuged for 30 minutes using a centrifuge. The supernatant was removed, and the precipitated DNA was washed with 200 μl of 70% ethanol. Using a centrifuge, the DNA was centrifuged for 10 minutes. The supernatant was removed, and the precipitated

DNA was dried. The DNA was dissolved in deionized water to a final concentration of 1 μg/μl.

Into 10 μl of gold particles were mixed 1μ of a DNA solution, 10 μl of 12.5 M CaCl₂, and 0.1 M spermidine, and the mixture was stirred for 3 minutes using a vortex mixer. After the mixture was rapidly centrifuged with a centrifuge, the supernatant was removed. The precipitate of gold particles was resuspended in 30 μl of 70% ethanol, this was rapidly centrifuged with a centrifuge again, and the supernatant was removed. The precipitate of gold particles was resuspended in 10 μl of 100% ethanol.

The precipitated unc-119 mutant (0.5 ml) was resuspended in 3 ml of a M9 buffer. 300 μl of the suspension was taken out with a pipette, and spread on a 4.5 cm agar medium.

Gold particles coated with the DNA were resuspended using pipetting and a vortex mixer. Into a stainless Sweeney filter holder 13 mm (manufactured by Millipore) was filled 10 μl of a gold particle suspension, and this was attached to a gene firing device utilizing a pressure of a helium gas. Using this device, gold particles were accelerated with a gas pressure (8 bar, for 10 milliseconds), and fired into the nematode. This was allowed to stand for 1 hour until the nematode was recovered, and the nematode was recovered with a M9 buffer, and transferred to a 9 cm agar medium.

Culturing was performed on an agar medium at 20° C. for 12 days. After culturing for 12 days, a feed was depleted, and the nematode was brought into the fasting state. The transformant could survive the term of this fasting state via a resistant larva, but the non-transformant could not survive.

By this selection, a survivable transformant was screened. It was confirmed that a selected nematode exhibits the same movement as that of a wild type. The transformants were isolated, and transferred into separate petri dishes one by one. These nematodes were in a F2 generation seen from the P0 generation fired with gold particles. In order to identify a line in which the introduced gene is inserted into a genome, a next generation of a F2 generation was bred. Letting the F2 generation to be a parent, when a ratio of a transformant in a next generation (F3) was 75% (heterozygote-type) or 100% (homozygote-type), this was deemed to be a line in which the introduced gene is incorporated into a genome. UG2295 (genotype: unc-119; bgls1289 [myo-2:: aphid seizure (D2); unc-119-unc-119]) was selected, and the presence of pGBB014 (myo-2:: aphid seizure (D2)) was confirmed by PCR.

Like this, in a procedure by which the introduced gene is randomly incorporated into a genome, since there is a possibility that a line in which a plurality of plasmids are inserted, or a line in which an introduced gene ga is inserted into a gene generating an unnecessary phenotype may appear, UG2295 was back-mated to a wild-type nematode. Via two times of back mating, finally, UG2346 (genotype: +/+; bgls1289 [myo-2:: aphid seizure (D2); unc-119-unc-119]) was acquired.

By the same method, it is possible to make a transformant in which an introduced gene is incorporated into a genome also regarding D1, E1 and E2.

### Example 10

### Electropharyngeogram of Recombinant Nematode

By recording a electropharyngeogram using UG2242 which is a transient transformed line, and UG2346 which is a stable transformed line, it was confirmed that the introduced cotton aphid seizure gene was expressed in a functional form.

An imago of a nematode reared at 20° C. was prepared. Using Dent's saline (composition in mM; D-glucose 10 mM, HEPES 10 mM, NaCl 140 mM, KCl 6 mM, CaCl₂ 3 mM, MgCl₂ 1 mM, pH 7.4) containing 20 mM serotonin promoting pumping as a chamber internal solution, the nematode was placed into the chamber. Aspiration was performed until the Dent's saline which is a chamber internal solution was contacted with an electrode in a micropipette. After mild aspiration, a head of the nematode was inserted into a tip of a pipette for recording. Data was obtained with an amplifier (Multiclamp 700-A, Axon instruments) and an A/D converter (Digidata 1322A, Axon instruments).

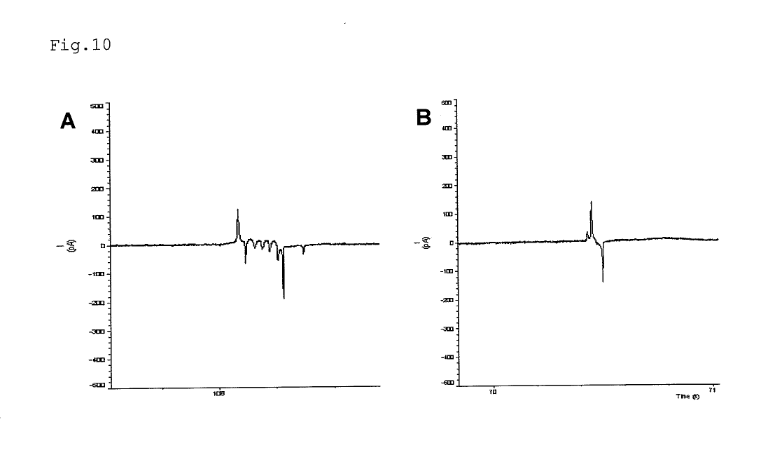

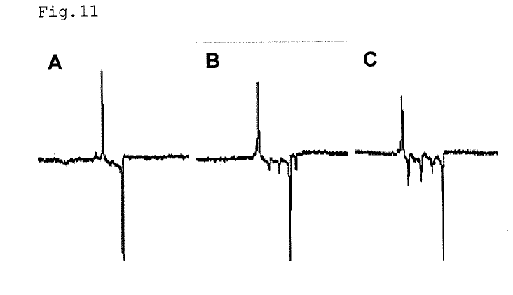

An electropharyngeogram of UG2234 (genotype: pha-1 (e2123ts) III; bgEx1253 [pha-1; myo-3:: gfp]) which is a control line is shown in FIG. 10A. On the other hand, an electropharyngeogram of a recombinant nematode UG2242 (genotype: pha-1 (e2123ts) III; bgEx1259 [pha-1; myo-2:: ahid seizure (D2); myo-3:: gfp]) with cotton aphid seizure D2 introduced therein is shown in FIG. 10B. As compared with UG2234 which is a control line, in UG2242 with cotton aphid seizure D2 introduced therein, the characteristic change that, in an electropharyngeogram, a lasting time of an action potential is decreased, and a length of a potential waveform is decreased was caused. A lasting time of an action potential was 200±50 ms in UG2234 which is a control line, while the lasting time was decreased to 50 ms in UG2242 with cotton aphid seizure D2 introduced therein. Also in UG2346 which is a stable transformed line, a similar result was obtained.

In order to demonstrate that an activity of the introduced cotton aphid seizure can be pharmacologically regulated in a recombinant nematode expressing cotton aphid seizure, and confirm that the recombinant nematode serves as a tool utilized in screening of a compound, UG2242 with cotton aphid seizure D2 introduced therein was treated with clofilium acting on both of ERG/EAG as a blocker. When an electropharyngeogram was recorded, before clofilium treatment (FIG. 11A), a length of a potential waveform was significantly decreased, but when 5 μM clofilium treatment was performed for 5 minutes (FIG. 11B), a length of a potential waveform was significantly increased, and became the same potential waveform as that of a wild-type after 15 minutes (FIG. 11C). This showed that clofilium can block cotton aphid seizure expressed in the recombinant nematode, and suitability of the recombinant nematode is pharmacologically demonstrated.

By a similar method, it is possible to confirm that D1, E1 and E2 are expressed in a functional form, in the recombinant nematode.

### Example 11

### Selection of Compound which Inhibits Feeding Behavior Activity of Recombinant Nematode

In the pharynx of a gene-altered nematode, change in electric activity in cotton aphid seizure influences on an action of the pharynx, that is, intake of food. Therefore, the ability to change an activity of cotton aphid seizure can be measured as the ability to change intake of food, of the gene-recombinant nematode.

Intake of food of the nematode was measured by a method of adding a fluorescent dye precursor to a medium. Specifically, since the fluorescent dye precursor orally taken by the nematode is enzymatically converted into a fluorescent dye in the gut, and a fluorescent signal emitted by the gene-recombinant nematode is increased, intake of food of the nematode can be measured as a fluorescent signal emitted by the nematode.

For measuring the activity to change a feeding behavior activity of a nematode expressing cotton aphid seizure in a functional form, a fluorescent dye precursor was added to a liquid medium containing the nematode expressing cotton aphid seizure in a functional form, and intake of the fluorescent dye precursor orally ingested by the nematode was detected as a fluorescent signal emitted by the nematode. When intake of the fluorescent dye precursor was defined as a feeding behavior activity, an influence of a test substance on a feeding behavior activity was assessed as an influence, on an activity of cotton aphid seizure.

As the nematode expressing cotton aphid seizure in a functional form, the recombinant nematode UG2346 produced in Example 9 was used and, as the fluorescent dye precursor, calcein AM (manufactured by Molecular Probes) was added. A fluorescent substance calcein which is converted from calcein AM was measured at excitation of 485 nm and emission of 535 nm, and a feeding behavior activity of the nematode was calculated.

For the activity measurement, a feeding behavior activity of the nematode expressing cotton aphid seizure in a functional form when clofilium dissolved in DMSO to a final concentration of 30 μM was contained, was measured. In addition, a feeding behavior activity of a nematode expressing cotton aphid seizure in a functional form when DMSO was contained in place of clofilium, was measured. Then, a ratio (%) of a feeding behavior activity measured value of the nematode when clofilium dissolved in DMSO was contained relative to a feeding behavior activity measured value of the nematode when DMSO was contained in place of clofilium was calculated, and was adopted as an inhibition degree (%).

In addition, a feeding behavior activity of a nematode expressing cotton aphid seizure in a functional form when clofilium dissolved in DMSO to a final concentration of 100 μM, 30 μM, 10 μM, 3 μM, 1 μM, 0.3 μM, 0.1 μM, and 0.03 μM, respectively, was contained, was measured. From the results at respective concentrations in the test compound, IC50 (μM) was calculated using a concentration dependency test analysis software XL fit (manufactured by IDBS). The results are shown in Table 6 in Example 11 together with the results of Example 12.

By a similar method, it is possible to select a compound having the ability to inhibit an activity of cotton aphid seizure among a plurality of compounds.

### Example 12

### Pesticidal Activity Test

A sterilized artificial feed having the following composition (Table 5) was prepared. Then, according to the same manner as that of the method described in Handbook of Insect Rearing Vol. 1 (Elsevier Science Publishers 1985) pp. 35 to pp. 36 except that a test compound dissolved in DMSO to a final concentration of 50 ppm was added at 0.5% volume of the artificial feed, and components were mixed, Aphis gossypii was reared. Six days after rearing, the number of surviving Aphis gossypii was investigated, and an entity exhibiting a significant controlling value (e.g. controlling value of 30% or more) was determined to have pesticidal activity by obtaining a controlling value by the following equation.

Controlling value(%)={1−(Cb×Tai)/(Cai×Tb)}×100

Letters in the equation represent the following meanings.

Cb: Number of surviving worms before treatment in non-treated section
Cai: Number of surviving worms at observation in non-treated section
Tb: Number of surviving worms before treatment in treated section
Tai: Number of surviving worms at observation in treated section

Results are shown in Table 6 in Example 12 together with results of Example 11.

TABLE 5

| Amino acid | (mg/100 ml) | Vitamins | (mg/100 ml) |
|---|---|---|---|
| L-alanine | 100.0 | Ascorbic acid | 100.0 |
| L-arginine | 275.0 | Biotin | 0.1 |
| L-asparagine | 550.0 | Calcium | 5.0 |
| | | pantothenate | |
| L-aspartic acid | 140.0 | Choline chloride | 50.0 |
| L-cysteine | 40.0 | Inositol | 50.0 |
| (hydrochloride) | | | |
| L-glutamic acid | 140.0 | Nicotinic acid | 10.0 |
| L-glutamine | 150.0 | Thiamine | 2.5 |
| L-glycine | 80.0 | | |
| L-histidine | 80.0 | Others | (mg/100 ml) |
| L-isoleucine | 80.0 | Sucrose | 12500.0 |
| L-leucine | 80.0 | Dipotassium | 1500.0 |
| | | hydrogen phosphate | |
| L-lysine | 120.0 | Magnesium sulfate | 123.0 |
| (hydrochloride) | | | |
| L-methionine | 80.0 | Cupric chloride | 0.2 |
| L-phenylalanine | 40.0 | Ferric chloride | 11.0 |
| L-proline | 80.0 | Manganese chloride | 0.4 |
| L-serine | 80.0 | Zinc sulfate | 0.8 |
| | | (anhydrous) | |
| L-threonine | 140.0 | | |

| | | |
|---|---|---|
| L-tryptophan | 80.0 | Adjusted to pH 6.8 |
| L-tyrosine | 40.0 | |
| L-valine | 80.0 | |

TABLE 6

| | Result of Example 11 | |
|---|---|---|
| | Activity to inhibit feeding | |
| | behavior of nematode | |
| | functionally expressing | Result of Example 12 |
| | cotton aphid seizure | Result of determination of |
| Compound name | (IC50, (μM)) | insecticidal activity |
| clofilium | 4.0 | Having insecticidal activity |

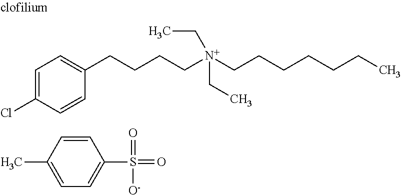

### INDUSTRIAL APPLICABILITY

According to the present invention, it becomes possible to provide a more target-based approach of screening agricultural chemicals, whereby compounds are screened against a specific target with intent of chemically interfering with the target site to control insects or other pest organisms.

1. A method for assaying the pesticidal activity of a test substance, comprising:
(1) a first step of measuring the feeding behavior activity of a nematode that expresses a voltage-gated potassium channel selected from among the following group A in a form functional as an ion channel in a system in which the nematode contacts with a test substance, and
(2) a second step of assessing the pesticidal activity of the test substance based on a difference obtained by comparing the feeding behavior activity measured in the first step with the feeding behavior activity of the nematode in a system containing no test substance;
<Group A>
(a) a protein comprising the amino acid sequence of SEQ ID NO: 1, 3, 5 or 7, or a partial sequence thereof functioning as an ion channel,
(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,
(c) a protein having the voltage-gated potassium channel activity and comprising an amino acid sequence having 60% or more sequence identity with the amino acid sequence of SEQ ID NO: 1, 3, 5 or 7,
(d) a protein comprising an amino acid sequence encoded by the nucleotide sequence of SEQ ID NO: 2, 4, 6 or 8,
(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,
(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,
(g) a protein comprising an amino acid sequence of an insect ERG-type voltage-gated potassium channel,
(h) a protein comprising an amino acid sequence of a cotton aphid ERG-type voltage-gated potassium channel, and
(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14.

2. A method for assaying the pesticidal activity of a test substance, comprising:
(1) a first step of measuring the electrophysiological activity of a voltage-gated potassium channel of a cell that expresses a voltage-gated potassium channel selected from among the following group A in a form functional as an ion channel in a system in which the cell contact with a test substance, and
(2) a second step of assessing the pesticidal activity of the test substance based on a difference obtained by comparing the electrophysiological activity of the voltage-gated potassium channel measured in the first step with the electrophysiological activity of the voltage-gated potassium channel of the cell in a system containing no test substance:
<Group A>
(a) a protein comprising the amino acid sequence of SEQ ID NO: 1, 3, 5 or 7, or a partial sequence thereof functioning as an ion channel,
(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,
(c) a protein having the voltage-gated potassium channel activity and comprising an amino acid sequence having 60% or more sequence identity with the amino acid sequence of SEQ ID NO: 1, 3, 5 or 7,
(d) a protein comprising an amino acid sequence encoded by the nucleotide sequence of SEQ ID NO: 2, 4, 6 or 8,
(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,
(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,
(g) a protein comprising an amino acid sequence of an insect ERG-type voltage-gated potassium channel,
(h) a protein comprising an amino acid sequence of a cotton aphid ERG-type voltage-gated potassium channel, and
(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14.

3. A method for screening a pesticidal substance, which comprises selecting a substance having the pesticidal activity that is evaluated by the method of claim 1.

4. A pesticidal agent which comprises a substance selected by the method of claim 3 or an agriculturally acceptable salt thereof as an active ingredient.

5. An agent that modulates physiological condition of pests, wherein the agent has an ability to modulate the activity of an insect voltage-gated potassium channel.

6. The agent according to claim 5, wherein the voltage-gated potassium channel is an insect voltage-gated potassium channel from the EAG family.

7. The agent according to claim 6, wherein the voltage-gated potassium channel is an insect ERG-type voltage-gated potassium channel.

8. The agent according to claim 7, wherein the voltage-gated potassium channel is a cotton aphid ERG-type voltage-gated potassium channel.

9. The agent according to claim 5, wherein the agent is a pesticidal agent.

10. The agent according to claim 5, wherein the ability to modulate the activity of an insect voltage-gated potassium channel is an ability to modulate the feeding behavior activity of a nematode that expresses the insect voltage-gated potassium channel in a form functional as an ion channel, or an ability to modulate the electrophysiological activity of the insect voltage-gated potassium channel in a cell that expresses the voltage-gated potassium channel in a form functional as an ion channel.

11. A pesticidal agent which comprises a substance that has an ability to modulate the activity of an insect voltage-gated potassium channel or an agriculturally acceptable salt of the substance as an active ingredient.

12. The pesticidal agent according to claim 11, wherein the substance has an ability to modulate the feeding behavior activity of a nematode which expresses the insect voltage-gated potassium channel in a form functional as an ion channel.

13. The pesticidal agent according to claim 12, wherein the substance has an ability to inhibit the feeding behavior activity of a nematode that expresses the insect voltage-gated potassium channel in a form functional as an ion channel, wherein in the presence of the substance of 30 micro M or more the feeding behavior activity is lower than that in the absence of the substance.

14. The pesticidal agent according to claim 12, wherein the substance has an ability to inhibit the feeding behavior activity of a nematode that expresses the insect voltage-gated potassium channel in a form functional as an ion channel, wherein an effective concentration of the substance at which the feeding behavior activity is reduced by 50% is 100 μM or lower.

15. The pesticidal agent according to claim 12, wherein the substance has an ability to activate the feeding behavior activity of a nematode that expresses the insect voltage-gated potassium channel in a form functional as an ion channel, wherein in the presence of said substance of 30 micro M or more the feeding behavior activity is higher than that in the absence of the substance.

16. The pesticidal agent according to claim 12, wherein the substance has an ability to activate the feeding behavior activity of a nematode that expresses the insect voltage-gated potassium channel in a form functional as an ion channel, wherein an effective concentration of the substance at which the feeding behavior activity is increased by 50% is 100 μM or lower.

17. The pesticidal agent according to claim 11, wherein the substance has an ability to modulate the electrophysiological activity of the insect voltage-gated potassium channel in a cell that expresses the voltage-gated potassium channel in a form functional as an ion channel.

18. A method for controlling pests which comprises applying an effective amount of the pesticidal agent of claim 4 to the pest, habitat of the pest or plant to be protected from the pest.

19. A method for controlling pests which comprises:
identifying a substance having the pesticidal activity that is evaluated by the method of claim 1, and
bringing the identified pesticidal substance into contact with the pest.

20. A voltage-gated potassium channel comprising an amino acid sequence selected from among the following group B:
<Group B>
(a) the amino acid sequence of SEQ ID NO: 1, 3, 5 or 7,
(b) an amino acid sequence which has the voltage-gated potassium channel activity and which has deletion, addition or substitution of one or more amino acids in the amino acid sequence of SEQ ID NO: 1, 3, 5 or 7,
(c) an amino acid sequence which has the voltage-gated potassium channel activity and which has sequence identity of 60% or more to the amino acid sequence of SEQ ID NO: 1, 3, 5 or 7,
(d) an amino acid sequence encoded by the nucleotide sequence of SEQ ID NO: 2, 4, 6 or 8,
(e) an amino acid sequence which has the voltage-gated potassium channel activity and which is encoded by a nucleotide sequence that has sequence identity of 75% or more to the nucleotide sequence of SEQ ID NO: 2, 4, 6 or 8,
(f) an amino acid sequence which has the voltage-gated potassium channel activity and which is encoded by a polynucleotide, wherein said polynucleotide hybridizes under a stringent condition to a polynucleotide comprising a nucleotide sequence complementary to the nucleotide sequence of SEQ ID NO: 2, 4, 6 or 8,
(g) an amino acid sequence of an insect ERG-type voltage-gated potassium channel,
(h) an amino acid sequence of a cotton aphid ERG-type voltage-gated potassium channel, and
(i) an amino acid sequence which has the voltage-gated potassium channel activity and which is encoded by a polynucleotide amplifiable by PCR employing a cDNA of cotton aphid as a template, and using as primers a polynucleotide comprising the nucleotide sequence of SEQ ID NO: 11, 13 or 15, and a polynucleotide comprising the nucleotide sequence of SEQ ID NO: 12 or 14.

21. Use of a nematode expressing an insect voltage-gated potassium channel in a form functional as an ion channel, as a research tool for providing an indicator to evaluate pesticidal activity.

22. Use of a nematode expressing the voltage-gated potassium channel of claim 20 in a form functional as an ion channel, as a research tool for providing an indicator to evaluate pesticidal activity.

23. Use of a cell expressing the voltage-gated potassium channel of claim 20 in a form functional as an ion channel, as a research tool for providing an indicator to evaluate pesticidal activity.

24. A polynucleotide comprising a nucleotide sequence encoding an amino acid sequence of the voltage-gated potassium channel of claim 20.

25. The polynucleotide according to claim 24, wherein the polynucleotide comprising the nucleotide sequence of SEQ ID NO: 2, 4, 6 or 8.

26. A polynucleotide comprising a nucleotide sequence complementary to a nucleotide sequence of the polynucleotide of claim 24.

27. A polynucleotide comprising a partial nucleotide sequence of the polynucleotide of claim 24, or a nucleotide sequence complementary to the partial nucleotide sequence.

28. The polynucleotide according to claim 27, wherein the polynucleotide comprising the nucleotide sequence any of SEQ ID NO: 11 to 15.

29. A method of obtaining a polynucleotide comprising a nucleotide sequence encoding an amino acid sequence of a voltage-gated potassium channel, which comprises: a step of amplifying a desired polynucleotide by polymerase chain reaction using the polynucleotide of claim 27 as a primer, a step of identifying the amplified desired polynucleotide, and a step of recovering the identified polynucleotide.

30. A method of obtaining a polynucleotide comprising a nucleotide sequence encoding an amino acid sequence of a voltage-gated potassium channel, which comprises: a step of detecting a desired polynucleotide by hybridization using the polynucleotide of claim 26 as a probe, a step of identifying the detected desired polynucleotide, and a step of recovering the identified polynucleotide.

31. A cyclic polynucleotide comprising the polynucleotide of claim 24 that is operably linked to a promoter expressible in a host organism or a host cell.

32. A circular polynucleotide comprising the polynucleotide of claim 24 that is operably linked to a promoter from a nematode.

33. The circular polynucleotide according to claim 32, wherein the promoter from a nematode is a promoter of a myo-2 gene.

34. The circular polynucleotide according to claim 31, wherein the circular polynucleotide has a replication origin for autonomous replication in a host cell.

35. A process for producing a circular polynucleotide, which comprises ligating the polynucleotide of claim 24 to a vector.

36. A transformant in which the polynucleotide of claim 24 has been introduced therein.

37. A cell transiently expressing an insect voltage-gated potassium channel, in which a transcription product of the polynucleotide of claim 24 has been introduced therein.

38. The transfromant according to claim 36, wherein the transformant is a transformed Escherichia coli.

39. The transformant according to claim 36, wherein the transformant is a transformed nematode.

40. The transformant according to claim 39, wherein the nematode is Caenorhabdtis elegans.

41. A process for producing a transformant, which comprises introducing the polynucleotide of claim 24 into a host cell.

42. A process for producing a voltage-gated potassium channel, which comprises a step of culturing the transformant of claim 36 and recovering the produced voltage-gated potassium channel.

43. Use of the voltage-gated potassium channel of claim 20 as a research tool.

44. The use according to claim 43, wherein the research tool is a research tool for screening a pesticidal agent.

45. A method of measuring an ability of a test substance to modulate the activity of an insect voltage-gated potassium channel, comprising:
(1) a first step of measuring the electrophysiological activity of a voltage-gated potassium channel of a cell that expresses a voltage-gated potassium channel selected from among the following group A in a form functional as an ion channel in a system in which the cell contact with a test substance, and
(2) a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the cell in a system containing no test substance;
<Group A>
(a) a protein comprising the amino acid sequence of SEQ ID NO: 1, 3, 5 or 7, or a partial sequence thereof functioning as an ion channel,
(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,
(c) a protein having the voltage-gated potassium channel activity and comprising an amino acid sequence having 60% or more sequence identity with the amino acid sequence of SEQ ID NO: 1, 3, 5 or 7,
(d) a protein comprising an amino acid sequence encoded by the nucleotide sequence of SEQ ID NO: 2, 4, 6 or 8,
(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,
(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,
(g) a protein comprising an amino acid sequence of an insect ERG-type voltage-gated potassium channel,
(h) a protein comprising an amino acid sequence of a cotton aphid ERG-type voltage-gated potassium channel, and
(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14.

46. The method according to claim 45, wherein the cell is an oocyte from Xenopus laevis.

47. A method of measuring an ability of a test substance to modulate an activity of an insect voltage-gated potassium channel, comprising:
(1) a first step of measuring the electrophysiological activity of a voltage-gated potassium channel of a nematode that expresses a voltage-gated potassium channel selected from among the following group A in a form functional as an ion channel in a system in which the nematode contact with a test substance, and
(2) a second step of assessing the ability of the test substance to modulate the activity of an insect voltage-gated potassium channel based on a difference obtained by comparing the electrophysiological activity of the voltage-gated potassium channel measured in the first step with the electrophysiological activity of the voltage-gated potassium channel of the nematode in a system containing no test substance;
<Group A>
(a) a protein comprising the amino acid sequence of SEQ ID NO: 1, 3, 5 or 7, or a partial sequence thereof functioning as an ion channel,
(b) a protein having the voltage-gated potassium channel activity and comprising an amino acid sequence in which one or a plurality of amino acids are deleted, added or substituted in the amino acid sequence of SEQ ID NO: 1, 3, 5 or 7 or a partial sequence thereof functioning as an ion channel,
(c) a protein having the voltage-gated potassium channel activity and comprising an amino acid sequence having 60% or more sequence identity with the amino acid sequence of SEQ ID NO: 1, 3, 5 or 7,
(d) a protein comprising an amino acid sequence encoded by the nucleotide sequence of SEQ ID NO: 2, 4, 6 or 8,
(e) a protein having the voltage-gated potassium channel activity and comprising an amino acid sequence encoded by a nucleotide sequence having 75% or more sequence identity with the nucleotide sequence of SEQ ID NO: 2, 4, 6 or 8,
(f) a protein having the voltage-gated potassium channel activity and comprising an amino acid sequence encoded by a polynucleotide which hybridizes under a stringent condition with a polynucleotide complementary to a polynucleotide that comprises the nucleotide sequence of SEQ ID NO: 2, 4, 6 or 8,
(g) a protein comprising an amino acid sequence of an insect ERG-type voltage-gated potassium channel,
(h) a protein comprising an amino acid sequence of a cotton aphid ERG-type voltage-gated potassium channel, and
(i) a protein having the voltage-gated potassium channel activity and comprising an amino acid sequence encoded by a polynucleotide that is amplifiable by PCR employing a cDNA of cotton aphid as a template and using as primers a polynucleotide comprising the nucleotide sequence of SEQ ID NO: 11, 13 or 15, and a polynucleotide comprising the nucleotide sequence of SEQ ID NO: 12 or 14;

48. The method according to claim 47, wherein the nematode is Caenorhabdtis elegans.

49. A system which comprises: a means for inputting/accumulating/managing data information related to an ability to change an activity of a voltage-gated potassium channel derived from an insect having a test substance, a means for inquiring/retrieving the data information based on a desired condition, and a means for displaying/outputting the inquired/retrieved result, regarding the test substance.


SEQUENCE LISTING (15 sequences)

SEQ ID NO: 1 - protein, 996 aa, Aphis gossypii
MELQQIQSPIITVLSLGADILPEYKLQSPKIHKWTILHYSPFKAVWDWIILLLVIYTAIFTPYFAAFLLNEPHFKGPRPLRFSFEDPLVVVDFFVDITFIIDIAINFRTTYVNKNDEVVSAPSKIALNYFRGWFLIDLVAAIPFDLLIAGSSAETDETTTLIGLLKTARLLRLVRVARKIDRYSEYGAAVLLLLMATFALIAHWLACIWYAIGNAERRHVDSKVGWLDILANDTHQYYSTNNTGGPSIRSRYITALYFTFSSLTSVGFGNVAPNTDAEKAFTICVMLVGSLMYASIFGNVSAIIQRLYSGTARYHTQMLRVREFIRFHQIPNPLRQRLEEYFQHAWTYTNGIDMNSVLKGFPECLQADICLHLNRNLLKNCSAFNGASPGCLRALSLKFRTTHAPPGDTLVHQGDVLTSLYFISRGSIEILKDNVVMAILSKNDIFGENPCKHATIGKSCCNVRALTYCDLHKIDRNDLLEVLDLYPEFYHSFKENLEITYYMRDETQVGVDPRAGRFSRKIRSRSSSQEPIGGTANIGATKKYYHSFHRGVSESCIGPSEPLRDDTNENDNVMSEFNNNEKYAEGHGILEFSTDKAGQDVTPMNLKFDDEAIQNRVSTLNSISGMFSQLKRSVTDIRLHGLSQKPHGRDEENGKPGNSGGLKTSTSHKHLFASQCNENQPLLEHQLPISESSALHQQHKEISSEHTSVQNMSAAGFSTTASSHYGTPTAQSPLHSACKAQTPPFPTTSMSVIADNTTVITPPSPQRVITCSQCGHSDVHYRIDRLSRQLDSLELSVSSDIKTILHILKHKNGDFLTTESRPDGCRLRPSPIKKTSSIPTSDHLPRPTLAVKFRSSSQDEMKSDSRCRQQASPVRQSSDPQATIPRRSYSQPHDLCQTQQSRFVRDTPSANLFDVSNVTTATDATPPVASLKRQQFASLRCTTSCSSDEGNVIEFTPLAPLARLDSLHEMDTSETQSRTSSTTASPTHQQQQGTGF

SEQ ID NO: 2 - DNA, 2991 bp, Aphis gossypii
atggagcttcaacaaattcagtccccgataatcacagtgttatcgctgggcgcggatatactacccgagtacaagctccaatctcccaagatccacaagtggacgatactgcactacagcccgttcaaggcggtatgggattggataatactgttgttggtcatctacaccgccatattcacgccgtatttcgccgctttcctgttaaacgaaccgcacttcaaaggtccccggccgctgaggttcagtttcgaagatccgctggtggtggtcgatttttttgtggatataacgttcataatagacatcgccattaacttccgtaccacttacgtgaataagaacgacgaggtggtatcggcgcccagcaagattgcgttgaattatttccgtggatggttcctgatcgatttagttgccgcaatacctttcgaccttttgatcgctggatcgtcggctgagacggacgagaccacgacactcatcggccttctgaagaccgccaggctgctgaggctggtccgggtagcgcggaagatcgacaggtattccgaatacggtgctgcggtgttgcttctgttgatggctacgttcgcactgatagcccactggctggcgtgcatatggtacgcgatcgggaacgcggagcggcgtcacgtggacagcaaagtggggtggctggacatattagccaacgacacgcatcagtattactcgaccaacaacacgggtggaccgagcataaggtcgaggtacattacagcgctgtacttcacattcagctcgttgacttccgtggggtttggaaacgtagcgcccaataccgacgcggagaaagcgttcaccatatgcgtgatgctcgtcggatctctcatgtatgccagtatattcggtaatgtatccgcgattattcaaaggttatactccggaacagccaggtatcatacccaaatgctcagagttcgagaatttattagatttcatcaaataccaaatccattaaggcaaaggctcgaagaatattttcaacatgcttggacatacactaatggtattgacatgaatagtgtacttaagggtttccctgaatgtttgcaagccgatatttgtctccatcttaacaggaatttattgaaaaattgttcagctttcaatggtgcatcaccaggatgtttgagagccctatctttgaaattccggacgacgcatgcaccgcccggtgatactttagtgcatcaaggagacgtgctcacctctttgtatttcatatcacgtggttccattgaaatattaaaagataatgtagtaatggccatattaagtaaaaacgatatttttggggaaaatccatgcaagcatgctacgattggaaaatcatgttgcaacgtaagagcattaacttactgcgatcttcacaaaatcgatagaaacgatttgctcgaagtcttagatttgtatccagaattttatcattcatttaaagaaaatctggagataacatactacatgagagatgaaacacaagtaggtgtggatcctagagctggcaggtttagtagaaaaattaggtcaagatctagttcacaagaaccaattggtggcacagccaacataggtgcgacaaaaaagtattaccatagctttcatcgaggggtatctgaaagttgtattggaccgtcagagccactaagagatgatacaaatgagaacgacaacgtcatgtctgagtttaataacaacgagaagtatgcagaaggacatggaattttagagttttctactgataaagctggccaagatgtaactccgatgaatttaaaatttgatgacgaagcaatacagaatcgagtatcaaccttaaattcaatttccggcatgttcagccaactcaaacgtagtgtgacagacattcgcttgcatggcctatcgcaaaaaccacatggcagggacgaggaaaatgggaaacctggaaactctggtggtttaaagacatcgacatctcataaacacttgttcgcatctcaatgtaacgagaatcaaccacttcttgaacatcagctacctatttcagagtcttctgcgctgcatcaacaacacaaagaaattagtagtgaacacaccagtgttcaaaacatgtcagcagctggtttcagtacgactgcatcgtcacattatggaaccccaacagcccagagccccttacattccgcttgtaaagcacaaaccccaccatttcccaccacatctatgtctgtgatcgctgataatacgactgtgataacgcctccatcaccgcaacgtgtcataacatgttcgcagtgtgggcactccgatgtgcattatcgtatagatagactatccaggcagctcgatagcttggaactgtccgtctctagtgatatcaaaacgatattgcatatacttaaacataagaacggcgactttttgacaacagaatctcggccagatggatgtagattacggccgtcgcctataaagaagacatcgagtataccaacaagcgatcacttgcctagacccacgttggccgtaaaatttagaagctcatcacaagatgaaatgaaaagcgatagccggtgtcgacagcaagcgtctccagtaaggcaatcgtctgatcctcaagccaccattccaaggcggagttattcccagcctcacgatctctgtcagactcaacagagccgttttgtaagggatacgccatcggctaacctgtttgatgtatccaacgtgacgactgcaacagacgccacaccaccggttgccagtctgaaacgacaacagttcgccagtttgcggtgtacgactagttgttccagcgacgaaggcaacgtaattgagttcactccactggcgccactcgccaggctagactcgttgcacgaaatggatacatccgagacgcagagtcgcacgagcagtacgacggccagtccaacgcaccaacaacaacaagggaccggtttctag

SEQ ID NO: 3 - protein, 964 aa, Aphis gossypii
MELQQIQSPIITVLSLGADILPEYKLQSPKIHKWTILHYSPFKAVWDWIILLLVIYTAIFTPYFAAFLLNEPHFKGPRPLRFSFEDPLVVVDFFVDITFIIDIAINFRTTYVNKNDEVVSAPSKIALNYFRGWFLIDLVAAIPFDLLIAGSSAETDETTTLIGLLKTARLLRLVRVARKIDRYSEYGAAVLLLLMATFALIAHWLACIWYAIGNAERRHVDSKVGWLDILANDTHQYYSTNNTGGPSIRSRYITALYFTFSSLTSVGFGNVAPNTDAEKAFTICVMLVGSLMYASIFGNVSAIIQRLYSGTARYHTQMLRVREFIRFHQIPNPLRQRLEEYFQHAWTYTNGIDMNSVLKGFPECLQADICLHLNRNLLKNCSAFNGASPGCLRALSLKFRTTHAPPGDTLVHQGDVLTSLYFISRGSIEILKDNVVMAILSKNDIFGENPCKHATIGKSCCNVRALTYCDLHKIDRNDLLEVLDLYPEFYHSFKENLEITYYMRDETQVGVDPRAGRFSRKIRSRSSSQEPIGGTANIGATKKYYHSFHRGVSESCIGPSEPLRDDTNENDNVMSEFNNNEKYAEGHGILEFSTDKAGQDVTPMNLKFDDEAIQNRVSTLNSISGNSGGLKTSTSHKHLFASQCNENQPLLEHQLPISESSALHQQHKEISSEHTSVQNMSAAGFSTTASSHYGTPTAQSPLHSACKAQTPPFPTTSMSVIADNTTVITPPSPQRVITCSQCGHSDVHYRIDRLSRQLDSLELSVSSDIKTILHILKHKNGDFLTTESRPDGCRLRPSPIKKTSSIPTSDHLPRPTLAVKFRSSSQDEMKSDSRCRQQASPVRQSSDPQATIPRRSYSQPHDLCQTQQSRFVRDTPSANLFDVSNVTTATDATPPVASLKRQQFASLRCTTSCSSDEGNVIEFTPLAPLARLDSLHEMDTSETQSRTSSTTASPTHQQQQGTGF

SEQ ID NO: 4 - DNA, 2895 bp, Aphis gossypii
atggagcttcaacaaattcagtccccgataatcacagtgttatcgctgggcgcggatatactacccgagtacaagctccaatctcccaagatccacaagtggacgatactgcactacagcccgttcaaggcggtatgggattggataatactgttgttggtcatctacaccgccatattcacgccgtatttcgccgctttcctgttaaacgaaccgcacttcaaaggtccccggccgctgaggttcagtttcgaagatccgctggtggtggtcgatttttttgtggatataacgttcataatagacatcgccattaacttccgtaccacttacgtgaataagaacgacgaggtggtatcggcgcccagcaagattgcgttgaattatttccgtggatggttcctgatcgatttagttgccgcaatacctttcgaccttttgatcgctggatcgtcggctgagacggacgagaccacgacactcatcggccttctgaagaccgccaggctgctgaggctggtccgggtagcgcggaagatcgacaggtattccgaatacggtgctgcggtgttgcttctgttgatggctacgttcgcactgatagcccactggctggcgtgcatatggtacgcgatcgggaacgcggagcggcgtcacgtggacagcaaagtggggtggctggacatattagccaacgacacgcatcagtattactcgaccaacaacacgggtggaccgagcataaggtcgaggtacattacagcgctgtacttcacattcagctcgttgacttccgtggggtttggaaacgtagcgcccaataccgacgcggagaaagcgttcaccatatgcgtgatgctcgtcggatctctcatgtatgccagtatattcggtaatgtatccgcgattattcaaaggttatactccggaacagccaggtatcatacccaaatgctcagagttcgagaatttattagatttcatcaaataccaaatccattaaggcaaaggctcgaagaatattttcaacatgcttggacatacactaatggtattgacatgaatagtgtacttaagggtttccctgaatgtttgcaagccgatatttgtctccatcttaacaggaatttattgaaaaattgttcagctttcaatggtgcatcaccaggatgtttgagagccctatctttgaaattccggacgacgcatgcaccgcccggtgatactttagtgcatcaaggagacgtgctcacctctttgtatttcatatcacgtggttccattgaaatattaaaagataatgtagtaatggccatattaagtaaaaacgatatttttggggaaaatccatgcaagcatgctacgattggaaaatcatgttgcaacgtaagagcattaacttactgcgatcttcacaaaatcgatagaaacgatttgctcgaagtcttagatttgtatccagaattttatcattcatttaaagaaaatctggagataacatactacatgagagatgaaacacaagtaggtgtggatcctagagctggcaggtttagtagaaaaattaggtcaagatctagttcacaagaaccaattggtggcacagccaacataggtgcgacaaaaaagtattaccatagctttcatcgaggggtatctgaaagttgtattggaccgtcagagccactaagagatgatacaaatgagaacgacaacgtcatgtctgagtttaataacaacgagaagtatgcagaaggacatggaattttagagttttctactgataaagctggccaagatgtaactccgatgaatttaaaatttgatgacgaagcaatacagaatcgagtatcaaccttaaattcaatttccggaaactctggtggtttaaagacatcgacatctcataaacacttgttcgcatctcaatgtaacgagaatcaaccacttcttgaacatcagctacctatttcagagtcttctgcgctgcatcaacaacacaaagaaattagtagtgaacacaccagtgttcaaaacatgtcagcagctggtttcagtacgactgcatcgtcacattatggaaccccaacagcccagagccccttacattccgcttgtaaagcacaaaccccaccatttcccaccacatctatgtctgtgatcgctgataatacgactgtgataacgcctccatcaccgcaacgtgtcataacatgttcgcagtgtgggcactccgatgtgcattatcgtatagatagactatccaggcagctcgatagcttggaactgtccgtctctagtgatatcaaaacgatattgcatatacttaaacataagaacggcgactttttgacaacagaatctcggccagatggatgtagattacggccgtcgcctataaagaagacatcgagtataccaacaagcgatcacttgcctagacccacgttggccgtaaaatttagaagctcatcacaagatgaaatgaaaagcgatagccggtgtcgacagcaagcgtctccagtaaggcaatcgtctgatcctcaagccaccattccaaggcggagttattcccagcctcacgatctctgtcagactcaacagagccgttttgtaagggatacgccatcggctaacctgtttgatgtatccaacgtgacgactgcaacagacgccacaccaccggttgccagtctgaaacgacaacagttcgccagtttgcggtgtacgactagttgttccagcgacgaaggcaacgtaattgagttcactccactggcgccactcgccaggctagactcgttgcacgaaatggatacatccgagacgcagagtcgcacgagcagtacgacggccagtccaacgcaccaacaacaacaagggaccggtttctag

SEQ ID NO: 5 - protein, 1085 aa, Aphis gossypii
MGDDRDDLEMPLMPRLDDGGDGRKASVVVTVVPRDGFAYGASSSLRLVKQPSRPPSSCSSVDGRTSNDRKKSSANGGFDIKSPSKSAHSSGSIKTFKTSAQVLSLGADILPEYKLQSPKIHKWTILHYSPFKAVWDWIILLLVIYTAIFTPYFAAFLLNEPHFKGPRPLRFSFEDPLVVVDFFVDITFIIDIAINFRTTYVNKNDEVVSAPSKIALNYFRGWFLIDLVAAIPFDLLIAGSSAETDETTTLIGLLKTARLLRLVRVARKIDRYSEYGAAVLLLLMATFALIAHWLACIWYAIGNAERRHVDSKVGWLDILANDTHQYYSTNNTGGPSIRSRYITALYFTFSSLTSVGFGNVAPNTDAEKAFTICVMLVGSLMYASIFGNVSAIIQRLYSGTARYHTQMLRVREFIRFHQIPNPLRQRLEEYFQHAWTYTNGIDMNSVLKGFPECLQADICLHLNRNLLKNCSAFNGASPGCLRALSLKFRTTHAPPGDTLVHQGDVLTSLYFISRGSIEILKDNVVMAILSKNDIFGENPCKHATIGKSCCNVRALTYCDLHKIDRNDLLEVLDLYPEFYHSFKENLEITYYMRDETQVGVDPRAGRFSRKIRSRSSSQEPIGGTANIGATKKYYHSFHRGVSESCIGPSEPLRDDTNENDNVMSEFNNNEKYAEGHGILEFSTDKAGQDVTPMNLKFDDEAIQNRVSTLNSISGMFSQLKRSVTDIRLHGLSQKPHGRDEENGKPGNSGGLKTSTSHKHLFASQCNENQPLLEHQLPISESSALHQQHKEISSEHTSVQNMSAAGFSTTASSHYGTPTAQSPLHSACKAQTPPFPTTSMSVIADNTTVITPPSPQRVITCSQCGHSDVHYRIDRLSRQLDSLELSVSSDIKTILHILKHKNGDFLTTESRPDGCRLRPSPIKKTSSIPTSDHLPRPTLAVKFRSSSQDEMKSDSRCRQQASPVRQSSDPQATIPRRSYSQPHDLCQTQQSRFVRDTPSANLFDVSNVTTATDATPPVASLKRQQFASLRCTTSCSSDEGNVIEFTPLAPLARLDSLHEMDTSETQSRTSSTTASPTHQQQQGTGF

SEQ ID NO: 6 - DNA, 3258 bp, Aphis gossypii
atgggggatgaccgcgatgacctggaaatgcctctgatgccgcgactagacgacggtggcgatggccgaaaggcgtccgtggtggtgacggtggtgccgcgggacgggttcgcgtacggtgcgtcgtccagtctgcggctggtcaagcagccgtcgcggccgccgtcgtcgtgttcgtcggtcgacggccggacgtcgaacgaccggaagaagagctcggccaatggcgggttcgacatcaagtcgccgtccaagtcggcccattcttccgggtctatcaagactttcaagacttcggctcaggtgttatcgctgggcgcggatatactacccgagtacaagctccaatctcccaagatccacaagtggacgatactgcactacagcccgttcaaggcggtatgggattggataatactgttgctggtcatctacaccgccatattcacgccgtatttcgccgctttcctgttaaacgaaccgcacttcaaaggtccccggccgctgaggttcagtttcgaagatccgctggtggtggtcgatttttttgtggatataacgttcataatagacatcgccattaacttccgtaccacttacgtgaataagaacgacgaggtggtatcggcgcccagcaagattgcgttgaattatttccgtggatggttcctgatcgatttagttgccgcaatacctttcgaccttttgatcgctggatcgtcggctgagacggacgagaccacgacactcatcggccttctgaagaccgccaggctgctgaggctggtccgggtagcgcggaagatcgacaggtattccgaatacggtgctgcggtgttgcttctgttgatggctacgttcgcactgatagcccactggctggcgtgcatatggtacgcgatcgggaacgcggagcggcgtcacgtggacagcaaagtggggtggctggacatattagccaacgacacgcatcagtattactcgaccaacaacacgggtggaccgagcataaggtcgaggtacattacagcgctgtacttcacattcagctcgttgacttccgtggggtttggaaacgtagcgcccaataccgacgcggagaaagcgttcaccatatgcgtgatgctcgtcggatctctcatgtatgccagtatattcggtaatgtatccgcgattattcaaaggttatactccggaacagccaggtatcatacccaaatgctcagagttcgagaatttattagatttcatcaaataccaaatccattaaggcaaaggctcgaagaatattttcaacatgcttggacatacactaatggtattgacatgaatagtgtacttaagggtttccctgaatgtttgcaagccgatatttgtctccatcttaacaggaatttattgaaaaattgttcagctttcaatggtgcatcaccaggatgtttgagagccctatctttgaaattccggacgacgcatgcaccgcccggtgatactttagtgcatcaaggagacgtgctcacctctttgtatttcatatcacgtggttccattgaaatattaaaagataatgtagtaatggccatattaagtaaaaacgatatttttggggaaaatccatgcaagcatgctacgattggaaaatcatgttgcaacgtaagagcattaacttactgcgatcttcacaaaatcgatagaaacgatttgctcgaagtcttagatttgtatccagaattttatcattcatttaaagaaaatctggagataacatactacatgagagatgaaacacaagtaggtgtggatcctagagctggcaggtttagtagaaaaattaggtcaagatctagttcacaagaaccaattggtggcacagccaacataggtgcgacaaaaaagtattaccatagctttcatcgaggggtatctgaaagttgtattggaccgtcagagccactaagagatgatacaaatgagaacgacaacgtcatgtctgagtttaataacaacgagaagtatgcagaaggacatggaattttagaattttctactgataaagctggccaagatgtaactccgatgaatttaaaatttgatgacgaagcaatacagaatcgagtatcaaccttaaattcaatttccggcatgttcagccaactcaaacgtagtgtgacagacattcgcttgcatggcctatcgcaaaaaccacatggcagggacgaggaaaatgggaaacctggaaactctggtggtttaaagacatcgacatctcataaacacttgttcgcatctcaatgtaacgagaatcaaccacttcttgaacatcagctacctatttcagagtcttctgcgctgcatcaacaacacaaagaaattagtagtgaacacaccagtgttcaaaacatgtcagcagctggtttcagtacgactgcatcgtcacattatggaaccccaacagcccagagccccttacattccgcttgtaaagcacaaaccccaccatttcccaccacatctatgtctgtgatcgctgataatacgactgtgataacgcctccatcaccgcaacgtgtcataacatgttcgcagtgtgggcactccgatgtgcattatcgtatagatagactatccaggcagctcgatagcttggaactgtccgtctctagtgatatcaaaacgatattgcatatacttaaacataagaacggcgactttttgacaacagaatctcggccagatggatgtagattacggccgtcgcctataaagaagacatcgagtataccaacaagcgatcacttgcctagacccacgttggccgtaaaatttagaagctcatcacaagatgaaatgaaaagcgatagccggtgtcgacagcaagcgtctccagtaaggcaatcgtctgatcctcaagccaccattccaaggcggagttattcccagcctcacgatctctgtcagactcaacagagccgttttgtaagggatacgccatcggctaacctgtttgatgtatccaacgtgacgactgcaacagacgccacaccaccggttgccagtctgaaacgacaacagttcgccagtttgcggtgtacgactagttgttccagcgacgaaggcaacgtaattgagttcactccactggcgccactcgccaggctagactcgttgcacgaaatggatacatccgagacgcagagtcgcacgagcagtacgacggccagtccaacgcaccaacaacaacaagggaccggtttctag

SEQ ID NO: 7 - protein, 1053 aa, Aphis gossypii
MGDDRDDLEMPLMPRLDDGGDGRKASVVVTVVPRDGFAYGASSSLRLVKQPSRPPSSCSSVDGRTSNDRKKSSANGGFDIKSPSKSAHSSGSIKTFKTSAQVLSLGADILPEYKLQSPKIHKWTILHYSPFKAVWDWIILLLVIYTAIFTPYFAAFLLNEPHFKGPRPLRFSFEDPLVVVDFFVDITFIIDIAINFRTTYVNKNDEVVSAPSKIALNYFRGWFLIDLVAAIPFDLLIAGSSAETDETTTLIGLLKTARLLRLVRVARKIDRYSEYGAAVLLLLMATFALIAHWLACIWYAIGNAERRHVDSKVGWLDILANDTHQYYSTNNTGGPSIRSRYITALYFTFSSLTSVGFGNVAPNTDAEKAFTICVMLVGSLMYASIFGNVSAIIQRLYSGTARYHTQMLRVREFIRFHQIPNPLRQRLEEYFQHAWTYTNGIDMNSVLKGFPECLQADICLHLNRNLLKNCSAFNGASPGCLRALSLKFRTTHAPPGDTLVHQGDVLTSLYFISRGSIEILKDNVVMAILSKNDIFGENPCKHATIGKSCCNVRALTYCDLHKIDRNDLLEVLDLYPEFYHSFKENLEITYYMRDETQVGVDPRAGRFSRKIRSRSSSQEPIGGTANIGATKKYYHSFHRGVSESCIGPSEPLRDDTNENDNVMSEFNNNEKYAEGHGILEFSTDKAGQDVTPMNLKFDDEAIQNRVSTLNSISGNSGGLKTSTSHKHLFASQCNENQPLLEHQLPISESSALHQQHKEISSEHTSVQNMSAAGFSTTASSHYGTPTAQSPLHSACKAQTPPFPTTSMSVIADNTTVITPPSPQRVITCSQCGHSDVHYRIDRLSRQLDSLELSVSSDIKTILHILKHKNGDFLTTESRPDGCRLRPSPIKKTSSIPTSDHLPRPTLAVKFRSSSQDEMKSDSRCRQQASPVRQSSDPQATIPRRSYSQPHDLCQTQQSRFVRDTPSANLFDVSNVTTATDATPPVASLKRQQFASLRCTTSCSSDEGNVIEFTPLAPLARLDSLHEMDTSETQSRTSSTTASPTHQQQQGTGF

SEQ ID NO: 8 - DNA, 3162 bp, Aphis gossypii
atgggggatgaccgcgatgacctggaaatgcctctgatgccgcgactagacgacggtggcgatggccgaaaggcgtccgtggtggtgacggtggtgccgcgggacgggttcgcgtacggtgcgtcgtccagtctgcggctggtcaagcagccgtcgcggccgccgtcgtcgtgttcgtcggtcgacggccggacgtcgaacgaccggaagaagagctcggccaatggcgggttcgacatcaagtcgccgtccaagtcggcccattcttccgggtctatcaagactttcaagacttcggctcaggtgttatcgctgggcgcggatatactacccgagtacaagctccaatctcccaagatccacaagtggacgatactgcactacagcccgttcaaggcggtatgggattggataatactgttgctggtcatctacaccgccatattcacgccgtatttcgccgctttcctgttaaacgaaccgcacttcaaaggtccccggccgctgaggttcagtttcgaagatccgctggtggtggtcgatttttttgtggatataacgttcataatagacatcgccattaacttccgtaccacttacgtgaataagaacgacgaggtggtatcggcgcccagcaagattgcgttgaattatttccgtggatggttcctgatcgatttagttgccgcaatacctttcgaccttttgatcgctggatcgtcggctgagacggacgagaccacgacactcatcggccttctgaagaccgccaggctgctgaggctggtccgggtagcgcggaagatcgacaggtattccgaatacggtgctgcggtgttgcttctgttgatggctacgttcgcactgatagcccactggctggcgtgcatatggtacgcgatcgggaacgcggagcggcgtcacgtggacagcaaagtggggtggctggacatattagccaacgacacgcatcagtattactcgaccaacaacacgggtggaccgagcataaggtcgaggtacattacagcgctgtacttcacattcagctcgttgacttccgtggggtttggaaacgtagcgcccaataccgacgcggagaaagcgttcaccatatgcgtgatgctcgtcggatctctcatgtatgccagtatattcggtaatgtatccgcgattattcaaaggttatactccggaacagccaggtatcatacccaaatgctcagagttcgagaatttattagatttcatcaaataccaaatccattaaggcaaaggctcgaagaatattttcaacatgcttggacatacactaatggtattgacatgaatagtgtacttaagggtttccctgaatgtttgcaagccgatatttgtctccatcttaacaggaatttattgaaaaattgttcagctttcaatggtgcatcaccaggatgtttgagagccctatctttgaaattccggacgacgcatgcaccgcccggtgatactttagtgcatcaaggagacgtgctcacctctttgtatttcatatcacgtggttccattgaaatattaaaagataatgtagtaatggccatattaagtaaaaacgatatttttggggaaaatccatgcaagcatgctacgattggaaaatcatgttgcaacgtaagagcattaacttactgcgatcttcacaaaatcgatagaaacgatttgctcgaagtcttagatttgtatccagaattttatcattcatttaaagaaaatctggagataacatactacatgagagatgaaacacaagtaggtgtggatcctagagctggcaggtttagtagaaaaattaggtcaagatctagttcacaagaaccaattggtggcacagccaacataggtgcgacaaaaaagtattaccatagctttcatcgaggggtatctgaaagttgtattggaccgtcagagccactaagagatgatacaaatgagaacgacaacgtcatgtctgagtttaataacaacgagaagtatgcagaaggacatggaattttagaattttctactgataaagctggccaagatgtaactccgatgaatttaaaatttgatgacgaagcaatacagaatcgagtatcaaccttaaattcaatttccggaaactctggtggtttaaagacatcgacatctcataaacacttgttcgcatctcaatgtaacgagaatcaaccacttcttgaacatcagctacctatttcagagtcttctgcgctgcatcaacaacacaaagaaattagtagtgaacacaccagtgttcaaaacatgtcagcagctggtttcagtacgactgcatcgtcacattatggaaccccaacagcccagagccccttacattccgcttgtaaagcacaaaccccaccatttcccaccacatctatgtctgtgatcgctgataatacgactgtgataacgcctccatcaccgcaacgtgtcataacatgttcgcagtgtgggcactccgatgtgcattatcgtatagatagactatccaggcagctcgatagcttggaactgtccgtctctagtgatatcaaaacgatattgcatatacttaaacataagaacggcgactttttgacaacagaatctcggccagatggatgtagattacggccgtcgcctataaagaagacatcgagtataccaacaagcgatcacttgcctagacccacgttggccgtaaaatttagaagctcatcacaagatgaaatgaaaagcgatagccggtgtcgacagcaagcgtctccagtaaggcaatcgtctgatcctcaagccaccattccaaggcggagttattcccagcctcacgatctctgtcagactcaacagagccgttttgtaagggatacgccatcggctaacctgtttgatgtatccaacgtgacgactgcaacagacgccacaccaccggttgccagtctgaaacgacaacagttcgccagtttgcggtgtacgactagttgttccagcgacgaaggcaacgtaattgagttcactccactggcgccactcgccaggctagactcgttgcacgaaatggatacatccgagacgcagagtcgcacgagcagtacgacggccagtccaacgcaccaacaacaacaagggaccggtttctag

SEQ ID NO: 9 - DNA, 96 bp, Aphis gossypii
catgttcagccaactcaaacgtagtgtgacagacattcgcttgcatggcctatcgcaaaaaccacatggcagggacgaggaaaatgggaaacctgg

SEQ ID NO: 10 - DNA, 3292 bp, Aphis gossypii
gaatatttaatacgacaacgcaataatatatcgagcgacttctgcgtgcagcttttagggtgttatacgacgcgtatagtgtcgaaatatatgttttacatattgctattacatgacgaattacggtggcgacgacgttatagcgacgaatggagcttcaacaaattcagtccccgataatcacagtgttatcgctgggcgcggatatactacccgagtacaagctccaatctcccaagatccacaagtggacgatactgcactacagcccgttcaaggcggtatgggattggataatactgttgctggtcatctacaccgccatattcacgccgtatttcgccgctttcctgttaaacgaaccgcacttcaaaggtccccggccgctgaggttcagtttcgaagatccgctggtggtggtcgatttttttgtggatataacgttcataatagacatcgccattaacttccgtaccacttacgtgaataagaacgacgaggtggtatcggcgcccagcaagattgcgttgaattatttccgtggatggttcctgatcgatttagttgccgcaatacctttcgaccttttgatcgctggatcgtcggctgagacggacgagttgactcaggaaacacagaagcccgaagagaccacgacactcatcggccttctgaagaccgccaggctgctgaggctggtccgggtagcgcggaagatcgacaggtattccgaatacggtgctgcggtgttgcttctgttgatggctacgttcgcactgatagcccactggctggcgtgcatatggtacgcgatcgggaacgcggaacggcgtcacgtggacagcaaagtggggtggctggacatattagccaacgacacgcatcagtattactcgaccaacaacacgggtggaccgagcataaggtcgaggtacattacagcgctgtacttcacattcagctcgttgacttccgtggggtttggaaacgtagcgcccaataccgacgcggagaaagcgttcaccatatgcgtgatgctcgtcggatctctcatgtatgccagtatattcggtaatgtatccgcgattattcaaaggttatrctccggaacagccaggtatcatacccaaatgctcagagttcgagaatttattagatttcatcaaataccaaatccattaaggcaaaggctcgargaatattttcaacatgcttggacatacactaatggtattgacatgaatagtgtacttaagggtttccctgaatgtttgcaagccgatatttgtctccatcttaacaggaatttattgaaaaattgttcagctttcaatggtgcatcaccaggatgtttgagagccctatctttgaaattccggacgacgcatgcaccgcccggtgatactttagtgcatcaaggagacgtgctcacctctttgtatttcatatcacgtggttccattgaaatattaaaagataatgtagtaatggccatattaagtaaaaacgatatttttggggaaaatccatgcaagcatgctacgattggaaaatcatgttgcaacgtaagagcattaacttactgcgatcttcacaaaatcgatagaaacgatttgctcgaagtcttagatttgtatccagaattttatcattcatttaaagaaaatctggagataacatactacatgagagatgaaacacaagtaggtgtggatcctagagctggcaggtttagtagaaaaattaggtcaagatctagttcacaagaaccaattggtggcacagccaacataggtgcgacaaaaaagtattaccatagctttcatcgaggggtatctgaaagttgtattggaccgtcagagccactaagrgatgatacaaatgagaacgayaacgtcatgtctgagtttaataacaacgrgaagtatgcagaaggacatggaattytagaattttctactgataaagctggccaagatgtaactccgaygaatttaaaatttgatgacgaagcaatacagaatcgagtatcaaccttaaattcaatttccggcatgttcagccaactcaaacgtagtgtgacagacattcgcttgcatggcctatcgcaaaaaccacatggcagggacgaggaaaatgggaaacctggaaactctggtggtttaaagacatcgacatctcataaacacttgttcgcatctcartgtaacgagaatcaaccacttcttgaacatcagytacctatttcagagtcttctgcgctgcatcaacaacacaaagaaattagtagtgaacacaccagtgttcaaaacatgtcagcagctggtttcagtacgactgcatcgtcacattatggaaccccaacagcccagagccccttacattccgcttgtaaagcacaaaccccaccatttcccaccacatctatgtctgtgatygctgrwaatacgactgtgataacgsctccatcmccgcaacgtgtcataacatgttcgcagtgtgggcactccgatgtgcattatcgtatagatagactatccaggcagctcgatagcttggaactgtccgtctctagtgatatcaaaacgatattgcatatacttaaacataagaacggcgamtttttgacaacagaatctcggccagatggatgtagattacggccgtcgcctataaagaagacatcgagtataccaacaagcgatcacttgcctagacccacgttggccgtaaaatttagaagctcatcacaagatgaaatgaaaagcgatagccggtgtcgacagcaagcgtctccagtaaggcaatcgtctgatcctcaagccaccattccaaggcggagttattcccagcctcacgatctctgtcagactcaacagagccgttttgtaagggatacgccatcggctaacctgtttgatgtatccaacgtgacgactgcaacagacgccacaccaccggttgccagtctgaaacgacaacagttcgccagtttgcggtgtacgactagttgttccagcgacgaaggcaacgtaattgagttcactccactggcgccactcgccaggctagactcgttgcacgaaatggatacatccgagacgcagagtcgcacgagcagtacgacggccagtccaacgcaccaacaacaacaagggaccggtttctagcgtcaacacccactgacgagggcgcgatcatatattcacgagcttgtttttgactcttcgtttcttttttgttttctttattattattttttttttttacataattcacgaaaagctttgct

SEQ ID NO: 11 - DNA, 26 bp, Artificial Sequence
  Description of Artificial Sequence Synthetic primer
catgacgaattacggtggcgacgacg

SEQ ID NO: 12 - DNA, 24 bp, Artificial Sequence
  Description of Artificial Sequence Synthetic primer
tgatcgcgccctcgtcagtgggtg

SEQ ID NO: 13 - DNA, 31 bp, Artificial Sequence
  Description of Artificial Sequence Synthetic primer
gctagccatacgaattacggtggcgacgacg

SEQ ID NO: 14 - DNA, 30 bp, Artificial Sequence
  Description of Artificial Sequence Synthetic primer
ccatggtgatcgcgccctcgtcagtgggtg

SEQ ID NO: 15 - DNA, 30 bp, Artificial Sequence
  Description of Artificial Sequence Synthetic primer
gctagcatgggggatgaccgcgatgacctg